(19) United States
(12) Patent Application Publication
(10) Pub. No.: US 2023/0381341 A1
(43) Pub. Date: Nov. 30, 2023

(54) ADENO-ASSOCIATED VIRUSES FOR OCULAR DELIVERY OF GENE THERAPY

(71) Applicant: REGENXBIO INC., Rockville, MD (US)

(72) Inventors: Joseph Bruder, Gaithersburg, MD (US); Xu Wang, Falls Church, VA (US); Wei-Hua Lee, Darnestown, MD (US); Elad Firnberg, Baltimore, MD (US); Samantha Yost, Rockville, MD (US); Andrew Mercer, Poolesville, MD (US); Ye Liu, Clarksville, MD (US); Olivier Danos, Princeton, NJ (US); April R. Tepe, Laurel, MD (US)

(21) Appl. No.: 18/030,706

(22) PCT Filed: Oct. 7, 2021
(86) PCT No.: PCT/US21/54008

§ 371 Date: Apr. 6, 2023

Related U.S. Application Data
(60) Provisional application No. 63/088,982, filed on Oct. 7, 2020.
(60) Provisional application No. 63/187,197, filed on May 11, 2021.

(51) Int. Cl. A61K 48/00; C12N 15/86; A61P 27/02; A61K 9/00

(52) U.S. Cl. CPC A61K 48/0058; C12N 15/86; A61K 48/0041; A61P 27/02; A61K 9/0048; C12N 2750/14143; C12N 2750/14122; C12N 2750/14171

(57) ABSTRACT

The present invention relates to recombinant adeno-associated viruses (rAAVs) having capsid proteins that have a tropism for ocular tissue. The rAAVs have capsids that have enhanced or increased transduction of ocular tissues as compared to reference rAAVs. Such rAAVs may be useful in delivering transgenes encoding therapeutic proteins for the treatment of ocular disease.

### 1. FIELD OF THE INVENTION

Disclosed herein are recombinant adeno-associated viruses (rAAVs) having capsid proteins that target or have a tropism for, ocular tissue, and have enhanced delivery to ocular tissue, for example, relative to a reference capsid. In particular, provided are rAAV vectors having a capsid which is an AAV3B, AAVrh.73, AAVhu.26, AAV.hu.51, AAV9S454.Tfr3 or other capsid demonstrated to target one or more ocular tissues. Also provided capsid proteins that direct rAAVs to target tissues, and/or improve transduction of ocular tissues, including retinal tissue and RPE choroidal tissue, and deliver therapeutics for treating retinal diseases, in particular non-infectious uveitis.

### 2. BACKGROUND

The use of adeno-associated viruses (AAV) as gene delivery vectors is a promising avenue for the treatment of many unmet patient needs. Dozens of naturally occurring AAV capsids have been reported, and mining the natural diversity of AAV sequences in primate tissues has identified over a hundred variants, distributed in clades. AAVs belong to the parvovirus family and are single-stranded DNA viruses with relatively small genomes and simple genetic components. Without a helper virus, AAV establishes a latent infection. An AAV genome generally has a Rep gene and a Cap gene, flanked by inverted terminal repeats (ITRs), which serve as replication and packaging signals for vector production. The capsid proteins form capsids that carry genome DNA and can determine tissue tropism to deliver DNA into target cells.

Due to low pathogenicity and the promise of long-term, targeted gene expression, recombinant AAVs (rAAVs) have been used as gene transfer vectors, in which therapeutic sequences are packaged into various capsids. Such vectors have been used in preclinical gene therapy studies and over twenty gene therapy products are currently in clinical development. Recombinant AAVs, such as AAV2, have demonstrated desirable retinal cell transduction properties and clinical trials using recombinant AAV2 for treatment of ocular diseases are underway. Tropism for other ocular tissues is desirable depending upon the indication to be treated. Attempts to enhance ocular tissue tropism of rAAVs in human subjects have met with limited success.

There remains a need for rAAV vectors with enhanced tropism for ocular tissues, including particular ocular tissues, e.g., to delivery therapies in treating disorders associated with the eye, e.g. non-infectious uveitis. There also is a need for rAAV vectors with enhanced tissue-specific targeting and/or enhanced tissue-specific transduction to deliver therapies.

### 3. SUMMARY OF THE INVENTION

Provided are recombinant AAV particles that have capsid proteins that direct the rAAVs to target tissues. The capsid proteins promote ocular tissue targeting and/or cellular uptake and/or integration of the rAAV genome, including targeting the rAAV particles to anterior segment tissue (cornea, iris, ciliary body, Schlemm's canal and/or the trabecular meshwork), or posterior segment tissue (such as retinal or RPE-choroid tissue), or the optic nerve (orbital segment or cranial segment), and deliver therapeutics for treating ocular disorders. The rAAVs may have a transgene encoding a therapeutic protein for treating ocular disorders, and provided are methods of administering the rAAV for delivery to ocular tissue for treatment of an ocular disease or disorder. In embodiments, the rAAV has a capsid of an AAV serotype 1 (AAV1; SEQ ID NO: 59); AAV serotype 2 (AAV2; SEQ ID NO:60); AAV serotype 3 (AAV3; SEQ ID NO:61), AAV serotype 3B (AAV3B; SEQ ID NO:74), AAV serotype 4 (AAV4; SEQ ID NO:62); AAV serotype 5 (AAV5; SEQ ID NO:63); AAV serotype 6 (AAV6; SEQ ID NO:64); AAV serotype 7 (AAV7; SEQ ID NO:65); AAV serotype 8 (AAV8; SEQ ID NO:66); AAV serotype 9 (AAV9; SEQ ID NO:67); AAV serotype 9e (AAV9e; SEQ ID NO:68); AAV serotype rh.10 (AAVrh.10; SEQ ID NO:69); AAV serotype rh.20 (AAV.rh.20; SEQ ID NO:70); AAV serotype hu.37 (AAVhu.37; SEQ ID NO:71), AAV serotype rh39 (AAVrh.39; SEQ ID NO:73), AAV serotype rh73 (AAVrh.73; SEQ ID NO:75), AAV serotype rh.74 (AAVrh.74; SEQ ID NO:72 or SEQ ID NO:96), AAV serotype hu51 (AAVhu.51; SEQ ID NO:76), AAV serotype hu.21 (AAVhu.21; SEQ ID NO:77), AAV serotype hu.12 (AAVhu.12; SEQ ID NO:78), AAV serotype hu.26 (AAVhu.26; SEQ ID NO:79), AAV serotype rh.24 (AAVrh.24; SEQ ID NO:87), AAV serotype hu.38 (AAVhu.38; SEQ ID NO:88), AAV serotype rh.72 (AAVrh.72; SEQ ID NO:89), AAV serotype hu.56 (AAVhu.56; SEQ ID NO:86), AAV serotype cy.5 (AAVcy.5; SEQ ID NO:90), AAV serotype cy.6 (AAVcy.6; SEQ ID NO:91), AAV serotype rh.46 (AAVrh.46; SEQ ID NO:92), AAV serotype rh.13 (AAV.rh.13; SEQ ID NO:85), or AAV serotype rh.64.R1 (AAVrh.64.R1; SEQ ID NO:107), or the capsid is an engineered capsid having an insertion and/or one or more amino acid substitutions relative to one of the capsids disclosed herein, including, AAV9. S454-TFR3 (SEQ ID NO: 42), AAV8.BBB (A269S substitution (SEQ ID NO: 26)), AAV8.BBB.LD (A296S, 498 NNN/AAA 500; SEQ ID NO:27)), AAV8.Y703F (Y703F substitution in the amino acid sequence of SEQ ID NO:66, see FIG. 7 for numbering), AAV9.Y443F (Y443F substitution in the amino acid sequence of SEQ ID NO:67, see FIG. 7 for numbering), AAV9.Y6F (Y6F substitution in the amino acid sequence of SEQ ID NO:66, see FIG. 7 for numbering) (see FIG. 7 or Table 10).

In certain embodiments, the rAAV has a capsid of an AAV3B serotype, AAVrh.73 serotype, AAV.hu.26 serotype, AAVhu.51, AAVrh64R1 serotype or is AAV9.S454.TFR3. Certain rAAV capsids have a tropism for specific ocular tissue and may be used to target specific ocular tissues. In embodiments, rAAVs having an AAV3B or AAVrh.73 capsid may be administered to target the iris, retina, RPE choroid or sclera, and in certain embodiments, the ciliary body, Schlemm's canal, trabelcular meshwork or optic nerve (orbital and/or cranial segment). In embodiments, rAAVs having an AAV3B or AAVrh.73 capsid may be used to target the retina and/or RPE choroid tissue. In other embodiments, rAAVs having an AAVrh.73 capsid may be used to target the iris tissue, and in other embodiments, AAVhu.26 capsids may be used to target the ciliary body or the trabecular meshwork. In embodiments, the ciliary body and/or trabecular meshwork are targeted for treatment of glaucoma. AAV1 capsids may be used to target the trabecular meshwork or the sclera and AAV7 may be used to target the trabecular meshwork. In certain embodiments, the rAAV is administered in the absence of hyaluronic acid. The rAAV may be delivered by intravitreal, suprachoroidal, or intracameral administration and in certain embodiments the administration may be to a specific ocular tissue, such as to the, retina, retinal pigment epithelium, choroid, sclera or ciliary body.

Also provided are engineered capsid proteins that promote transduction of the rAAV in one or more tissues, including one or more cell types, upon systemic, intravenous, intracameral, suprachoroidal or intravitreal administration, wherein the capsid proteins comprise a peptide that is inserted into a surface-exposed variable region (VR) of the capsid, e.g. VR-I, VR-IV or VR-VIII, or after the first amino acid of VP2, e.g., immediately after residue 138 of the AAV9 capsid (amino acid sequence of SEQ ID NO:67) or immediately after the corresponding residue of another AAV capsid, or alternatively is engineered with one or more of the amino acid substitutions described herein, and transduction of the AAV having the engineered capsid in the at least one tissue, for example the anterior segment or the posterior segment, or both, is increased upon said administration compared to the transduction of the AAV having the corresponding unengineered capsid. In certain embodiments, transduction is measured by detection of transgene, such as GFP fluorescence. In particular embodiments, the rAAV having the engineered capsid transduced ocular tissue, including anterior segment or posterior segment tissues transduced ocular tissue, including anterior or posterior segments, by 1.1 fold, 1.5 fold, 2 fold, 3 fold, 5 fold, 6 fold, 7 fold, 8 fold, 9 fold or 10 fold greater than transduction by the reference AAV (the parental AAV serotype without the insertion).

In certain embodiments, provided are rAAVs incorporating the engineered capsids described herein, including rAAVs with genomes comprising a transgene of therapeutic interest. Packaging cells for producing the rAAVs described herein are provided. Method of treatment by delivery of, and pharmaceutical compositions comprising, the engineered rAAVs described herein are also provided. Also provided are methods of manufacturing the rAAVs with the engineered capsids described herein.

The invention is illustrated by way of examples infra describing the construction of engineered capsids and screening of capsids for tropism for ocular tissues after IV or IVT administration using barcoded rAAVs in mice and NHPs.

### 3.1. Embodiments

1. A method of delivering a transgene to an ocular tissue cell, said method comprising contacting said cell with an rAAV vector comprising a transgene encoding an ocular disease therapeutic operably linked to one or more regulatory elements that promote expression of the ocular disease therapeutic in the ocular tissue cell, wherein the rAAV has a capsid of AAV1 (SEQ ID NO: 59); AAV2 (SEQ ID NO:60); AAV3 SEQ ID NO:61); AAV3B (SEQ ID NO:74); AAV4 (SEQ ID NO:62); AAV5 (SEQ ID NO:63); AAV6 (SEQ ID NO:64); AAV7 (SEQ ID NO:65); AAV8 (SEQ ID NO:66); AAV9 (SEQ ID NO:67); AAV9e (SEQ ID NO:68); AAVrh.10 (SEQ ID NO:69); AAVrh.20 (SEQ ID NO:70); AAVhu.37 (SEQ ID NO:71); AAVrh39 (SEQ ID NO:73); AAV rh73 (SEQ ID NO:75); AAVrh.74 (SEQ ID NO:72 or SEQ ID NO:96); AAVhu.51 (SEQ ID NO:76); AAVhu.21 (SEQ ID NO:77); AAVhu.12 (SEQ ID NO:78); AAVhu.26 (SEQ ID NO:79); AAVrh.24 (SEQ ID NO:87); AAVhu.38 (SEQ ID NO:88); AAVrh.72 (SEQ ID NO:89); AAVhu.56 (SEQ ID NO:86); AAVcy.5 (SEQ ID NO:90); AAVcy.6 (SEQ ID NO:91); AAVrh.46 (SEQ ID NO:92); AAVrh.13 (SEQ ID NO:85); AAVrh.64.R1 (SEQ ID NO:107); AAV9.S454-TFR3 (SEQ ID NO: 42); AAV8.BBB (SEQ ID NO: 26); AAV8.BBB.LD (SEQ ID NO:27); AAV8.Y703F (Y703F substitution in the amino acid sequence of SEQ ID NO:66, see FIG. 7 for numbering); AAV9.Y443F (Y443F substitution in the amino acid sequence of SEQ ID NO:67, see FIG. 7 for numbering); or AAV9.Y6F (Y6F substitution in the amino acid sequence of SEQ ID NO:66, see FIG. 7 for numbering).

2. A method of delivering a transgene to ocular tissue, or an ocular tissue target cell or cellular matrix thereof, of a subject in need thereof, said method comprising administering to said subject an rAAV vector comprising a transgene encoding an ocular disease therapeutic operably linked to one or more regulatory elements that promote expression of said ocular disease therapeutic in said ocular tissue, wherein the rAAV has a capsid AAV1 (SEQ ID NO: 59); AAV2 (SEQ ID NO:60); AAV3 SEQ ID NO:61); AAV3B (SEQ ID NO:74); AAV4 (SEQ ID NO:62); AAV5 (SEQ ID NO:63); AAV6 (SEQ ID NO:64); AAV7 (SEQ ID NO:65); AAV8 (SEQ ID NO:66); AAV9 (SEQ ID NO:67); AAV9e (SEQ ID NO:68); AAVrh.10 (SEQ ID NO:69); AAVrh.20 (SEQ ID NO:70); AAVhu.37 (SEQ ID NO:71); AAVrh39 (SEQ ID NO:73); AAV rh73 (SEQ ID NO:75); AAVrh.74 (SEQ ID NO:72 or SEQ ID NO:96); AAVhu.51 (SEQ ID NO:76); AAVhu.21 (SEQ ID NO:77); AAVhu.12 (SEQ ID NO:78); AAVhu.26 (SEQ ID NO:79); AAVrh.24 (SEQ ID NO:87); AAVhu.38 (SEQ ID NO:88); AAVrh.72 (SEQ ID NO:89); AAVhu.56 (SEQ ID NO:86); AAVcy.5 (SEQ ID NO:90); AAVcy.6 (SEQ ID NO:91); AAVrh.46 (SEQ ID NO:92); AAVrh.13 (SEQ ID NO:85); AAVrh.64.R1 (SEQ ID NO:107); AAV9.S454-TFR3 (SEQ ID NO: 42); AAV8.BBB (SEQ ID NO: 26); AAV8.BBB.LD (SEQ ID NO:27); AAV8.Y703F (Y703F substitution in the amino acid sequence of SEQ ID NO:66, see FIG. 7 for numbering); AAV9.Y443F (Y443F substitution in the amino acid sequence of SEQ ID NO:67, see FIG. 7 for numbering); or AAV9.Y6F (Y6F substitution in the amino acid sequence of SEQ ID NO:66, see FIG. 7 for numbering).

3. The method of embodiment 1 or 2, wherein the capsid is an AAV3B serotype, AAVrh.73 serotype, AAV.hu.26 serotype, AAVhu.51, AAVrh64R1 serotype or AAV9.S454.TFR3 capsid.

4. The method of any of embodiments 1 to 3, in which the ocular tissue or ocular tissue target cell is a cornea tissue or cell, iris tissue or cell, ciliary body tissue or cell, Schlemm's canal tissue or cell, trabecular meshwork tissue or cell, retinal tissue or cell, RPE-choroid tissue or cell, or optic nerve cell.

5. The method of embodiment 4, wherein the ocular tissue or ocular tissue target cell is a retinal tissue or cell or an RPE-choroid tissue or cell.

6. The method of embodiment 5, wherein the capsid is an AAV3B or AAVrh.73 capsid.

7. The method of embodiment 1 to 6, wherein the ocular disease is non-infectious uveitis.

8. The method of embodiment 1 to 4, wherein the ocular disease is glaucoma.

9. The method of embodiment 8 wherein said rAAV targets the trabecular meshwork and/or the Schlemm's canal.

10. The method of embodiments 8 or 9 wherein the capsid is an AAV1 capsid, AAV2, AAV7 capsid, AAV3B capsid, AAV.hu.26 capsid, or AAV9.S454-TFR3 capsid.

11. The method of any of embodiments 1 to 10, wherein said rAAV vector is administered intravitreally, suprachoroidally, or intracamerally.

12. The method of any of embodiments 1 to 10 wherein said rAAV vector is administered systemically.

13. The method of any of embodiments 1 to 12, wherein provided said rAAV vector is administered in the absence of hyaluronic acid.

14. A pharmaceutical composition for use in delivering a transgene to an ocular tissue cell, said composition comprising an rAAV vector comprising a transgene encoding an ocular disease therapeutic operably linked to one or more regulatory elements that promote expression of the ocular disease therapeutic in the ocular tissue cell, wherein the rAAV has a capsid of AAV1 (SEQ ID NO: 59); AAV2 (SEQ ID NO:60); AAV3 SEQ ID NO:61); AAV3B (SEQ ID NO:74); AAV4 (SEQ ID NO:62); AAV5 (SEQ ID NO:63); AAV6 (SEQ ID NO:64); AAV7 (SEQ ID NO:65); AAV8 (SEQ ID NO:66); AAV9 (SEQ ID NO:67); AAV9e (SEQ ID NO:68); AAVrh.10 (SEQ ID NO:69); AAVrh.20 (SEQ ID NO:70); AAVhu.37 (SEQ ID NO:71); AAVrh39 (SEQ ID NO:73); AAV rh73 (SEQ ID NO:75); AAVrh.74 (SEQ ID NO:72 or SEQ ID NO:96); AAVhu.51 (SEQ ID NO:76); AAVhu.21 (SEQ ID NO:77); AAVhu.12 (SEQ ID NO:78); AAVhu.26 (SEQ ID NO:79); AAVrh.24 (SEQ ID NO:87); AAVhu.38 (SEQ ID NO:88); AAVrh.72 (SEQ ID NO:89); AAVhu.56 (SEQ ID NO:86); AAVcy.5 (SEQ ID NO:90); AAVcy.6 (SEQ ID NO:91); AAVrh.46 (SEQ ID NO:92); AAVrh.13 (SEQ ID NO:85); AAVrh.64.R1 (SEQ ID NO:107); AAV9.S454-TFR3 (SEQ ID NO: 42); AAV8.BBB (SEQ ID NO: 26); AAV5.BBB.LD (SEQ ID NO:27); AAV8.Y703F (Y703F substitution in the amino acid sequence of SEQ ID NO:66, see FIG. 7 for numbering); AAV9.Y443F (Y443F substitution in the amino acid sequence of SEQ ID NO:67, see FIG. 7 for numbering); or AAV9.Y6F (Y6F substitution in the amino acid sequence of SEQ ID NO:66, see FIG. 7 for numbering).

15. The pharmaceutical composition of embodiment 14, wherein the capsid is an AAV3B serotype, AAVrh.73 serotype, AAV.hu.26 serotype, AAVhu.51, AAVrh64R1 serotype or AAV9.S454.TFR3 capsid.

16. The pharmaceutical composition of embodiment 14 or 15, in which the ocular tissue or ocular tissue target cell is a cornea tissue or cell, iris tissue or cell, ciliary body tissue or cell, Schlemm's canal tissue or cell, trabecular meshwork tissue or cell, retinal tissue or cell, RPE-choroid tissue or cell, or optic nerve cell.

17. The pharmaceutical composition of embodiment 16, wherein the ocular tissue or ocular tissue target cell is a retinal tissue or cell or an RPE-choroid tissue or cell.

18. The pharmaceutical composition of embodiment 17, wherein the capsid is an AAV3B or AAVrh.73 capsid.

19. The pharmaceutical composition of embodiments 14 to 18, wherein the ocular disease is non-infectious uveitis.

20. The pharmaceutical composition of embodiment 14 to 18, wherein the ocular disease is glaucoma.

21. The pharmaceutical composition of embodiment 20 wherein said rAAV targets the trabecular meshwork and/or the Schlemm's canal.

22. The pharmaceutical composition of embodiments 20 or 21 wherein the capsid is an AAV3B serotype, AAVrh.73 serotype, AAV.hu.26 serotype, AAVhu.51, AAVrh64R1 serotype or AAV9.S454.TFR3 capsid.

23. The pharmaceutical composition of any of embodiments 14 to 22, wherein said rAAV vector is administered intravitreally, suprachoroidally, or intracamerally.

24. The method of any of embodiments 14 to 22 wherein said rAAV vector is administered systemically.

25. The method of embodiment 14 to 24, wherein provided said rAAV vector is administered in the absence of hyaluronic acid.

26. The method or pharmaceutical composition of any of the embodiments 1 to 25 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction in the target tissue, compared to a reference AAV capsid.

27. The method or pharmaceutical composition of any of embodiments 1 to 26 wherein the abundance of transgene RNA is 1.1-fold, 1.5-fold, 2-fold, 3-fold, 4-fold, 5-fold, 6-fold, 7-fold, 8-fold, 9-fold, or 10-fold greater in the target tissue compared to the abundance of transgene RNA from the reference AAV capsid.

28. The method or pharmaceutical composition of embodiments 26 or 27 where the reference AAV capsid is AAV2, AAV8 or AAV9

29. A method of treating an ocular disorder in a subject in need thereof, said method comprising administering a therapeutically effective amount of the pharmaceutical composition of any of embodiments 14-22 or 25.

30. The method of pharmaceutical composition of any of embodiments 1 to 29 wherein the ocular disease therapeutic is a VEGF fusion protein, such as aflibercept, an anti-VEGF antibody, or antigen-binding fragment thereof, such as, sevacizumab, ranibizumab, bevacizumab, or brolucizumab, an anti-kallikrein antibody, or antigen binding fragment thereof, such as lanadelumab, an anti-IL6 or anti-IL6R antibody, or antigen binding fragment thereof, such as satralizumab, sarilumab, siltuximab, clazakizumab, sirukumab, olokizumab, gerilimzumab, or tocilizumab, an anti-TNF antibody, or antigen binding fragment thereof, such as, adalimumab, infliximab, golimumab, or certolizumab-pegol, a TNF Receptor fusion protein, such as etanercept, an anti-C3 antibody, or antigen binding fragment thereof, such as, eculizumab, ravulizumab, or tesidolumab, or an anti-05 antibody, or antigen binding fragment thereof, such as NGM621.

31. A nucleic acid comprising a nucleotide sequence encoding the rAAV capsid protein of any of the above embodiments, or encoding an amino acid sequence sharing at least 80% identity therewith.

32. A packaging cell capable of expressing the nucleic acid of embodiment 31 to produce AAV vectors comprising the capsid protein encoded by said nucleotide sequence.

### 4. BRIEF DESCRIPTION OF THE FIGURES

FIG. 1 depicts sequence comparison of the capsid amino acid sequences including the VR-IV loop of the adeno-associated virus type 9 (AAV9 VR-IV) from residues L447 to R476, (with residues 451-459 bracketed) to corresponding to regions of other AAVs. Figure discloses SEQ ID NOS:49, 51-54, 50, and 55-58, respectively, in order of appearance.

FIG. 2 depicts a protein model of an AAV capsid structure, showing capsid variable regions VR-IV, VR-V and VR-VIII. The box highlights the loop region of VR-IV which provides surface-exposed amino acids as represented in the model.

FIG. 3 depicts high packaging efficiency (titer) in terms of genome copies per mL (GC/mL) of wild type AAV9 and eight (8) candidate modified rAAV9 vectors (1090, 1091, 1092, 1093, 1094, 1095, 1096, and 1097), where the candidate vectors each contain a FLAG insert immediately after different sites within AAV9s VR-IV, from residues 1451 to Q458, respectively. All vectors were packaged with luciferase transgene in 10 mL culture; error bars represent standard error of the mean.

FIG. 4 demonstrates surface exposure of 1 VR-IV loop FLAG inserts in each of eight (8) candidate modified rAAV9 vectors (1090, 1091, 1092, 1093, 1094, 1095, 1096, and 1097), confirmed by immunoprecipitation of packaged vectors by binding to anti-FLAG resin.

FIGS. 5A-5B depict transduction efficiency in Lec2 cells, transduced with capsid vectors carrying the luciferase gene (as a transgene), which were packaged into either wild type AAV9 (9-luc), or into each of eight (8) candidate modified (FLAG peptide inserted) rAAV9 vectors (1090, 1091, 1092, 1093, 1094, 1095, 1096, and 1097); transduction activity is expressed as percent luciferase activity, taking the activity of 9-luc as 100% (FIG. 5A), or as Relative Light Units (RLU) per microgram of protein (FIG. 5B).

FIGS. 6A-6E. FIG. 6A depicts a bar graph illustrating that insertions immediately after S454 of AAV9 of varying peptide length and composition may affect production efficiencies of AAV particles in a packaging cell. Ten peptides of varying composition and length were inserted after S454 within AAV9 VR-IV. qPCR was performed on harvested supernatant of transfected suspension HEK293 cells five days post-transfection. The results depicted in the bar graph demonstrate that the nature of the insertions affects the ability of AAV particles to be produced and secreted by HEK293 cells, and indicated by overall yields (titer). (Error bars represent standard error of the mean length of peptide, which is noted on the Y-axis in parenthesis.) FIGS. 6B-6E depict fluorescence images of transduced cell cultures of the following cell lines: (6B) Lec2 cell line (6C) HT-22 cell line, (6D) hCMEC/D3 cell line, and (6E) C2C12 cell line. AAV9 wild type and S454 insertion homing peptide capsids containing GFP transgene were used to transduce the noted cell lines. P1 vector was not included in images due to extremely low transduction efficiency, and P8 vector was not included due to low titer. AAV9.S454.FLAG showed low transduction levels in every cell type tested.

FIG. 7 depicts alignment of AAVs 1-9e, AAV3B, rh10, rh20, rh39, rh73, and rh74 version 1 and version 2, hu12, hu21, hu26, hu37, hu51 and hu53 capsid sequences with insertion sites for heterologous peptides after the initiation codon of VP2, and within or near variable region 1 (VR-I), variable region 4 (VR-IV), and variable region 8 (VR-VIII), all highlighted in grey; a particular insertion site within variable region eight (VR-VIII) of each capsid protein is shown by the symbol “#” (after amino acid residue 588 according to the amino acid numbering of AAV9). FIG. 7, top to bottom, shows the sequence of SEQ ID NOs:59, 60, 61, 94, 74, 62, 95, 63, 64, 65, 66, 67, 68, 69, 70, 73, 75, 72, 96, 78, 77, 79, 71, 76, 80, respectively.

FIG. 8 depicts copies of GFP (green fluorescent protein) transgene in mice brain cells, following administration of the AAV vectors: AAV9; AAV.PHP.eB, also referred to herein as AAV9e (AAV9 with the peptide TLAVPFK (SEQ ID NO:20) inserted between positions 588 and 589 and modifications A587D/A588G); AAV.hDyn (AAV9 with TLAAPFK (SEQ ID NO:1) between 588 and 589); AAV.PHP. S (AAV9 with the peptide QAVRTSL (SEQ ID NO:16) inserted between positions 588 and 589); and AAV.PHP.SH (AAV9 with the peptide QAVRTSH (SEQ ID NO:17) inserted between positions 588 and 589).

FIGS. 9A-9C depict the amino acid sequences for a recombinant AAV3B vector including a peptide insertion of LALGETTRPA (SEQ ID NO:9) between N588 and T589 (FIG. 9A, SEQ ID NO:97), between A267 and S268 of VR-III (FIG. 9B, SEQ ID NO:98), and between G454 and T455 of VR-IV (FIG. 9C, SEQ ID NO:99), each with the LALGETTRPA (SEQ ID NO:9) insert shown in bold.

FIGS. 10A-10C depict the amino acid sequences for a recombinant AAVrh73 vector including a peptide insertion of LALGETTRPA (SEQ ID NO:9) between N590 and T591 (FIG. 10A, SEQ ID NO:100), between T270 and N271 of VR-III (FIG. 10B, SEQ ID NO:101), and between G456 and G457 of VR-IV (FIG. 10C, SEQ ID NO:102), each with the LALGETTRPA (SEQ ID NO:9) insert shown in bold.

FIGS. 11A-11C depict the amino acid sequences for a recombinant AAV8 vector including a peptide insertion of LALGETTRPA (SEQ ID NO:9) between N590 and T591 (FIG. 11A), between A269 and T270 of VR-III (FIG. 11B), and between T453 and T454 of VR-IV (FIG. 11C), each with the LALGETTRPA (SEQ ID NO:9) insert shown in bold.

FIGS. 12A-12B depict an in vitro transwell assay for AAV vectors crossing a blood brain barrier (BBB) cell layer (FIG. 12A), and results showing that AAV.hDyn (indicated by inverted triangles) crosses the BBB cell layer of the assay faster than AAV9 (squares), as well as faster and to a greater extent than AAV2 (circles) (FIG. 12B).

FIG. 13 depicts results of Next Generation Sequencing (NGS) analysis of brain gDNA from mice to which pools of engineered and native capsids have been intravenously administered, revealing relative abundances in tissues of the mice of the different capsids in the pool. Three different pools were injected into mice. Dotted lines indicate which vectors were pooled together. Parental AAV9 was included in each pool as control (Pool 1: BC01, Pool 2: BC31, Pool 3: BC01). Bar codes for each capsid of the pool are listed in Table 4A-4C.

FIGS. 14A-14H depict an in vivo transduction profile of AAV.hDyn in female C57Bl/6 mice, showing copy number/microgram gDNA in naïve mice, or mice injected with either AAV9 or AAV.hDyn in brain (FIG. 14A), liver (FIG. 14B), heart (FIG. 14C), lung (FIG. 14D), kidney (FIG. 14E), skeletal muscle (FIG. 14F), sciatic nerve (FIG. 14G), and ovary (FIG. 14H), where AAV.hDyn shows increased brain bio-distribution compared to AAV9.

FIGS. 15A-15C depict distribution of GFP from AAV.hDyn throughout the brain, where images of immunohistochemical staining of brain sections from the striatum (FIG. 15A), hippocampus (FIG. 15B), and cortex (FIG. 15C) revealed a comprehensive transduction of the brain by the modified vector.

FIGS. 16A and B depict the anatomy of the eye. FIG. 16A depicts a cross section of the anterior of the eye and FIG. 16B depicts the anatomy of the entire eye.

FIGS. 17A and 17B depict an in vivo transduction analysis of gDNA (FIG. 17A) and RNA (FIG. 17B) isolated from the eyes of NHPs to which pools of engineered and native capsids have been administered by IVT, revealing relative abundances in cell types of the eye of the NHPs of the different capsids in the pool. Parental AAV2.7m8 was included in each pool as control and used to calculate relative abundunces.

FIGS. 18A-18C. Graphs representing relative abundance (relative to AAV9) of rAAV DNA and RNA expressed from transgene barcoded by capsid, for the nine most abundant capsid and controls AAV8 and AAV9, after IVT administration in NHPs in the ocular tissues, cornea (FIG. 18A), iris (FIG. 18B) or lens (FIG. 18C). Ordinary one-way ANOVA with post hoc Dunnett's multiple comparisons to AAV9 (#). P<0.0001 (****); p<0.001 (***); p<0.01 (**); p<0.05 (*). RNA transcribed from transgene not detectable in cornea or lens tissue

FIGS. 19A-19C. Graphs representing relative abundance (relative to AAV9) of rAAV DNA and RNA expressed from transgene barcoded by capsid, for the nine most abundant capsid and controls AAV8 and AAV9, after IVT administration in NHPs in ciliary body (FIG. 19A), Schlemm's canal (FIG. 19B) or trabecular meshwork (FIG. 19C). Ordinary one-way ANOVA with post hoc Dunnett's multiple comparisons to AAV9 (#). P<0.0001 (****); p<0.001 (***); p<0.01 (**); p<0.05 (*).

FIGS. 20A-20C. Graphs representing relative abundance (relative to AAV9) of rAAV DNA and RNA expressed from transgene barcoded by capsid, for the nine most abundant capsid and controls AAV8 and AAV9, after IVT administration in NHPs in retina (FIG. 20A), RPE-Choroid (FIG. 20B) or sclera (FIG. 20C). Ordinary one-way ANOVA with post hoc Dunnett's multiple comparisons to AAV9 (#). P<0.0001 (****); p<0.001 (***); p<0.01 (**); p<0.05 (*).

FIGS. 21A and 21B. Graphs representing relative abundance (relative to AAV9) of rAAV DNA and RNA expressed from transgene barcoded by capsid, for the nine most abundant capsid and controls AAV8 and AAV9, after IVT administration in NHPs in optic nerve (orbital segment) (FIG. 21A) or optical nerve (cranial segment) (FIG. 21B). Ordinary one-way ANOVA with post hoc Dunnett's multiple comparisons to AAV9 (#). P<0.0001 (****); p<0.001 (***); p<0.01 (**); p<0.05 (*). RNA transcribed from transgene not detectable in the optic nerve samples either the orbital or cranial segment.

FIGS. 22A and 22B. Graphs representing relative abundance (to AAV9) of rAAV DNA and RNA expressed from transgene barcoded by capsid, for the nine most abundant capsid and controls AAV8 and AAV9, after IVT administration in retinal tissue in mice (FIG. 22A) or in NHPs (FIG. 22B). Ordinary one-way ANOVA with post hoc Dunnett's multiple comparisons to AAV9 (#). P<0.0001 (****); p<0.001 (***); p<0.01 (**); p<0.05 (*).

FIGS. 23A and 23B. Graphs representing relative abundance (to AAV9) of rAAV DNA and RNA expressed from transgene barcoded by capsid, for the nine most abundant capsid and controls AAV8 and AAV9, after IVT administration in RPE-choroid relative to AAV8 or AAV9 in mice (FIG. 23A) or in NHPs (FIG. 23B). Ordinary one-way ANOVA with post hoc Dunnett's multiple comparisons to AAV9 (#). P<0.0001 (****); p<0.001 (***); p<0.01 (**); p<0.05 (*).

FIG. 24 shows a comparison of biodistribution of vectors in an rAAV vector pool in cynomolgus monkeys and mice after IVT Injection.

### 5. DETAILED DESCRIPTION

The inventors have identified capsids of adeno-associated viruses (AAVs) that promote targeting of recombinant AAV (rAAV) particles to ocular tissue, including transduction, cellular uptake, integration of the rAAV genome, and expression of transgenes delivered in the rAAV particle to a greater extent than an rAAV with a reference capsid, such as an AAV2, AAV8 or AAV9 capsid. Accordingly, provided are recombinant AAV particles that have capsid proteins that direct the rAAVs to target tissues. The capsid proteins promote ocular tissue targeting and/or cellular uptake and/or integration of the rAAV genome, including targeting the rAAV particles to anterior segment tissue (cornea, iris, ciliary body, Schlemm's canal and/or the trabecular meshwork), or posterior segment tissue (such as retinal or RPE-choroid tissue), or the optic nerve (orbital segment or cranial segment), and deliver therapeutics for treating ocular disorders. Included are rAAVs having capsid proteins engineered to include amino acid sequences that confer and/or enhance desired properties, such as ocular tissue targeting, transduction and integration of the rAAV genome relative to the parent, unengineered capsid or a reference capsid. The rAAVs may have a transgene encoding a therapeutic protein for treating ocular disorders, and provided are methods of administering the rAAV for delivery to ocular tissue for treatment of an ocular disease or disorder.

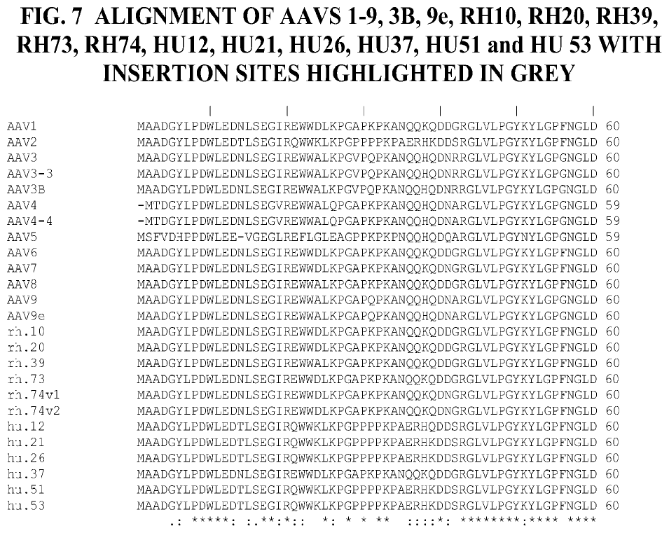

In embodiments, the rAAV has a capsid of an AAV serotype 1 (SEQ ID NO: 59); AAV serotype 2 (SEQ ID NO:60); AAV serotype 3 (SEQ ID NO:61), AAV serotype 3B (AAV3B) (SEQ ID NO:74), AAV serotype 4 (SEQ ID NO:62); AAV serotype 5 (SEQ ID NO:63); AAV serotype 6 (SEQ ID NO:64); AAV7 capsid (SEQ ID NO:65); AAV capsid (SEQ ID NO:66); AAV serotype 9 (SEQ ID NO:67); AAV serotype 9e (SEQ ID NO:68); AAV serotype rh10 (SEQ ID NO:69); AAV serotype rh20 (SEQ ID NO:70); and AAV serotype hu.37 (SEQ ID NO:71), AAV serotype rh39 (SEQ ID NO:73), AAV serotype rh73 (SEQ ID NO:75), AAV serotype rh74 (SEQ ID NO:72 or SEQ ID NO:96), AAV serotype hu51 (AAV.hu51) (SEQ ID NO:76), AAV serotype hu21 (AAV.hu21) (SEQ ID NO:77), AAV serotype hu12 (AAV.hu12) (SEQ ID NO:78), AAV serotype hu26 (AAV.hu26) (SEQ ID NO:79), AAV serotype rh.24 (SEQ ID NO:87), AAV serotype hu.38 (SEQ ID NO:88), AAV serotype rh.72 (SEQ ID NO:89), AAV serotype hu.56 (SEQ ID NO:86), AAV serotype cy.5 (SEQ ID NO:90), AAV serotype cy.6 (SEQ ID NO:91), AAV serotype rh.46 (SEQ ID NO:92), AAV serotype rh.13 (SEQ ID NO:85), or AAV serotype rh.64.R1 (SEQ ID NO:107) or the capsid is an engineered capsid having an insertion and/or one or more amino acid substitutions relative to one of the capsids disclosed herein, including, AAV9.S454-TFR3 (SEQ ID NO: 42), AAV8.BBB (A269S substitution (SEQ ID NO: 26)), AAV8.BBB.LD (A296S, 498 NNN/AAA 500; SEQ ID NO:27)), AAV8.Y703F (Y703F substitution in the amino acid sequence of SEQ ID NO:66, see FIG. 7 for numbering), AAV9.Y443F (Y443F substitution in the amino acid sequence of SEQ ID NO:67, see FIG. 7 for numbering), AAV9.Y6F (Y6F substitution in the amino acid sequence of SEQ ID NO:66, see FIG. 7 for numbering) (see FIG. 7 or Table 10).

Recombinant vectors comprising the capsid proteins also are provided, along with pharmaceutical compositions thereof, nucleic acids encoding the capsid proteins, and methods of making and using the capsid proteins and rAAV vectors having the ocular targeting capsids for targeted delivery, improved transduction and/or treatment of ocular disorders associated with the target ocular tissue. In particular, provided are compositions comprising rAAVs and methods of using capsid proteins to target rAAVs to ocular tissues, including the iris, cornea, ciliary body, Schlemm's canal, trabecular meshwork, RPE-choroid, the retina and optic nerve, and facilitate delivery of therapeutic agents for treating disorders of the eye.

In other embodiments, provided are rAAV vectors comprising a transgene which is an ophthalmic disease therapeutic and methods of treating an ocular disease or disorder in which the capsid is an AAV3B serotype, AAVrh.73 serotype, AAV.hu.26 serotype, AAVhu.51, AAVrh64R1 serotype or AAV9.S454.TFR3 capsid or other capsid shown herein to have tropism to an ocular tissue, including, the corneal, iris, lens ciliary body, Schlemm's canal, trabecular meshwork, retina, RPE-choroid, sclera, or optic nerve. In an embodiment, the eye disorder is non-infectious uveitis. In an embodiment, the eye disorder is glaucoma. Also provided are compositions comprising rAAVs comprising peptide insertions that target or home on target tissues, such as retina as well as methods of using same.

As used throughout. AAV “serotype” refers to an AAV having an immunologically distinct capsid, a naturally-occurring capsid, or an engineered capsid.

### 5.1. Definitions

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the terms “subject”, “host”, and “patient” are used interchangeably. As used herein, a subject is a mammal such as a non-primate (e.g., cows, pigs, horses, cats, dogs, rats etc.) or a primate (e.g., monkey and human), or, in certain embodiments, a human.

As used herein, the terms “therapeutic agent” refers to any agent which can be used in treating, managing, or ameliorating symptoms associated with a disease or disorder, where the disease or disorder is associated with a function to be provided by a transgene. As used herein, a “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

As used herein, the term “prophylactic agent” refers to any agent which can be used in the prevention, delay, or slowing down of the progression of a disease or disorder, where the disease or disorder is associated with a function to be provided by a transgene. As used herein, a “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

The “central nervous system” (“CNS”) as used herein refers to neural tissue reaches by a circulating agent after crossing a blood-brain barrier, and includes, for example, the brain, optic nerves, cranial nerves, and spinal cord. The CNS also includes the cerebrospinal fluid, which fills the central canal of the spinal cord as well as the ventricles of the brain.

As used throughout, AAV “serotype” refers to an AAV having an immunologically distinct capsid, a naturally-occurring capsid, or an engineered capsid.

### 5.2 Ocular Targeting Capsids and rAAVs

5.2.1 AAV Capsids with Tropism for Ocular Tissue

Identified herein are capsids that have a tropism for transduction and expression of transgenes in ocular tissue, including particular ocular tissues of the anterior or posterior segments of the eye, including, the cornea, the iris, the lens, the ciliary body, the Schlemm's canal, the trabecular meshwork, the retina, the RPE-Choroid, the sclera, or the optic nerve. The target tissue may also be a “retinal cell” type which include one or more of the cell types found in or near the retina, including amacrine cells, bipolar cells, horizontal cells, Muller glial cells, photoreceptor cells (e.g., rods and cones), retinal ganglion cells, retinal pigmented epithelium, and the like, and in particular, human photoreceptor cells (e.g., human cone cells and/or human rod cells), human horizontal cells, human bipolar cells, human amacrine cells, as well as human retina ganglion cells (e.g., midget cells, parasol cells, bistratified cells, giant retina ganglion cells, photosensitive ganglion cells, and/or Muller glia), endothelial cells in the inner limiting membrane, and/or human retinal pigment epithelial cells in the external limiting membrane.

In particular embodiments, provided are methods of delivering a transgene to ocular tissues, methods of treating an ocular disease and pharmaceutical compositions comprising an rAAV comprising a transgene encoding an ocular therapeutic, where the AAV has a capsid of AAV serotype 1 (SEQ ID NO:59); AAV serotype 2 (SEQ ID NO:60); AAV serotype 3 (SEQ ID NO:61), AAV serotype 3B (AAV3B) (SEQ ID NO:74), AAV serotype 4 (SEQ ID NO:62); AAV serotype 5 (SEQ ID NO:63); AAV serotype 6 (SEQ ID NO:64); AAV7 capsid (SEQ ID NO:65); AAV8 capsid (SEQ ID NO:66); AAV serotype 9 (SEQ ID NO:67); AAV serotype 9e (SEQ ID NO:68); AAV serotype rh10 (SEQ ID NO:69); AAV serotype rh20 (SEQ ID NO:70); and AAV serotype hu.37 (SEQ ID NO:71), AAV serotype rh39 (SEQ ID NO:73), AAV serotype rh73 (SEQ ID NO:75), AAV serotype rh74 (SEQ ID NO:72 or SEQ ID NO:96), AAV serotype hu51 (AAV.hu51) (SEQ ID NO:76), AAV serotype hu21 (AAV.hu21) (SEQ ID NO:77), AAV serotype hu12 (AAV.hu12) (SEQ ID NO:78), AAV serotype hu26 (AAV.hu26) (SEQ ID NO:79), AAV serotype rh.24 (SEQ ID NO:87), AAV serotype hu.38 (SEQ ID NO:88), AAV serotype rh.72 (SEQ ID NO:89), AAV serotype hu.56 (SEQ ID NO:86), AAV serotype cy.5 (SEQ ID NO:90), AAV serotype cy.6 (SEQ ID NO:91), AAV serotype rh.46 (SEQ ID NO:92), AAV serotype rh.13 (SEQ ID NO:85), or AAV serotype rh64 (SEQ ID NO:107) or variants thereof (see FIG. 7 or Table 10).

In specific embodiments, the capsid is an AAV3B serotype, AAVrh.73 serotype, AAV.hu.26 serotype, AAVhu.51, AAVrh64R1 serotype or AAV9.S454.TFR3 capsid.

Certain rAAV capsids have a tropism for specific ocular tissue and may be used to target specific ocular tissues. In embodiments, rAAVs having an AAV3B or AAVrh.73 capsid may be administered to target the iris, retina, RPE choroid or sclera, and in certain embodiments, the ciliary body, Schlemm's canal, trabelcular meshwork or optic nerve (orbital and/or cranial segment). In embodiments, rAAVs having an AAV3B or AAVrh.73 capsid may be used to target the retina and/or RPE choroid tissue. In other embodiments, rAAVs having an AAVrh.73 capsid may be used to target the iris tissue, and in other embodiments, AAVhu.26 capsids may be used to target the ciliary body or the trabecular meshwork. AAV1 capsids may be used to target the trabecular meshwork or the sclera and AAV7 may be used to target the trabecular meshwork.

The rAAV particles that have the ocular tissue targeting capsids described herein have enhanced targeting, transduction, genome integration, transgene mRNA transcription and/or transgene expression in ocular tissue compared to a reference rAAV particle having a reference capsid, for example an AAV2, AAV8 or AAV9 capsid. The enhancement may be in the ocular tissue overall or may be specifically the anterior segment tissue, posterior segment tissue or the optic nerve. In embodiments, the enhancement is in the iris, retina, RPE choroid, sclera, the ciliary body, Schlemm's canal, trabelcular meshwork, or optic nerve. The enhancement may be assessed as known in the art, for example in Examples 15 to 18 herein. In embodiments, the rAAV particles with an ocular tissue targeting capsid exhibit at least 1.1-fold, 1.5-fold, 2-fold, 3-fold, 4-fold, 5-fold, 6-fold, 7-fold, 8-fold, 9-fold, or 10-fold greater transduction or genome copy in the target tissue, compared to a reference AAV capsid, which may be AAV2, AAV8 or AAV9, and where the target tissue is ocular tissue, anterior ocular tissue, posterior ocular tissue, iris, retina, RPE choroid, sclera, the ciliary body, Schlemm's canal, trabelcular meshwork, or optic nerve. In embodiments, rAAV particles with an ocular tissue targeting capsid exhibit at least 1.1-fold, 1.5-fold, 2-fold, 3-fold, 4-fold, 5-fold, 6-fold, 7-fold, 8-fold, 9-fold, or 10-fold greater transgene mRNA or transgene protein expression in the target tissue compared to the abundance of transgene RNA or protein from the reference AAV capsid, which may be AAV2, AAV8 or AAV9, where the target tissue is ocular tissue, anterior ocular tissue, posterior ocular tissue, iris, retina, RPE choroid, sclera, the ciliary body, Schlemm's canal, trabelcular meshwork, or optic nerve.

5.2.2 Engineered rAAV Vectors with Peptide Insertions

Another aspect relates to capsid proteins, and rAAV particles comprising the capsid proteins which are modified by insertion of a peptide and/or one or more amino acid substitutions to confer or enhance ocular cell-homing properties, including enhanced transduction, AAV genome copy abundance or integration, transgene mRNA levels, or transgene protein expression. The modified capsid may target cells of the retina, including amacrine cells, bipolar cells, horizontal cells, Muller glial cells, photoreceptor cells (e.g., rods and cones), retinal ganglion cells, retinal pigmented epithelium, and the like, and in particular, human photoreceptor cells (e.g., human cone cells and/or human rod cells), human horizontal cells, human bipolar cells, human amacrine cells, as well as human retina ganglion cells (e.g., midget cells, parasol cells, bistratified cells, giant retina ganglion cells, photosensitive ganglion cells, and/or Muller glia), endothelial cells in the inner limiting membrane, and/or human retinal pigment epithelial cells in the external limiting membrane. The modified capsid may target other ocular tissues, including anterior segment tissues, including the iris, cornea, ciliary body, Schlemm's canal, trabecular meshwork, and posterior segment tissues, such as the retina or RPE-choroid, and optic nerve.

In embodiments, provided are modified capsids, and rAAV particles comprising the capsids, as listed in Table 10 or are described herein, including AAV9.S454-TFR3 (SEQ ID NO: 42), AAV8.BBB (A269S substitution (SEQ ID NO: 26)), AAV8.BBB.LD (A296S, 498 NNN/AAA 500; SEQ ID NO:27)), AAV8.Y703F (Y703F substitution in the amino acid sequence of SEQ ID NO:66, see FIG. 7 for numbering), AAV9.Y443F (Y443F substitution in the amino acid sequence of SEQ ID NO:67, see FIG. 7 for numbering), AAV9.Y6F (Y6F substitution in the amino acid sequence of SEQ ID NO:66, see FIG. 7 for numbering).

In particular embodiments, the peptide insertion for targeting ocular tissue is at least or consists of 4, 5, 6, 7, 8, 9, or 10 contiguous amino acids of RTIGPSV (SEQ ID NO:12). In one embodiment of particular interest, the peptide insertion comprises or consists of the amino acid sequence RTIGPSV (SEQ ID NO:12).

One aspect relates to a capsid protein of a recombinant adeno-associated virus (rAAV), the capsid protein engineered to target ocular tissue cells. In some embodiments the rAAV can comprise a peptide insertion, where the peptide insertion is surface exposed when packaged as an AAV particle. For example, the peptide insertion can be RTIGPSV (SEQ ID NO:12) or LALGETTRPA (SEQ ID NO:9) or any other peptide, for example peptide in Table which include SEQ ID NOs: 1-20, at least or consists of 4, 5, 6, 7, 8, 9, or 10 contiguous amino acids of RTIGPSV (SEQ ID NO:12) or LALGETTRPA (SEQ ID NO:9) or any other peptide of SEQ ID NO: 1-20. In some embodiments, the peptide insertion occurs within (i.e., between two amino acids without deleting any capsid amino acids) variable region IV (VR IV) of an AAV9 (SEQ ID NO: 118) capsid, or a corresponding region for another type AAV capsid, in particular, AAV3B, AAVrh73, AAV.hu.26, AAVhu.51, or AAVrh64R1 (see Table 10 and alignment in FIG. 7). In some embodiments, the peptide insertion occurs within (i.e., between two amino acids without deleting any capsid amino acids) variable region VIII (VR-VIII) of an AAV9 capsid, or a corresponding region of a capsid for another AAV type (see exemplary alignments in FIG. 7).

In the various embodiments, the rAAV capsids and/or insertion peptides direct the rAAV particles to target tissues, more specifically, the eye, including the anterior segment tissues or the posterior segment tissues, and/or promote rAAV uptake, transduction and/or genome integration. Also provided are nucleic acids encoding the engineered capsid proteins and variants thereof, packaging cells for expressing the nucleic acids to produce rAAV vectors, rAAV vectors further comprising a transgene, and pharmaceutical compositions of the rAAV vectors, as well as methods of using the rAAV vectors to deliver the transgene to a target cell type or target tissue of a subject in need thereof.

In the various embodiments, the rAAV capsid specifically recognizes and/or promotes transduction of ocular tissue, or for example, one or more specific cell types, such as within the target tissue, or cellular matrix thereof. In particular, the capsids target rAAVs to ocular tissues, including the iris, cornea, ciliary body, Schlemm's canal, trabecular meshwork, RPE-choroid, and optic nerve, and particularly, the retina.

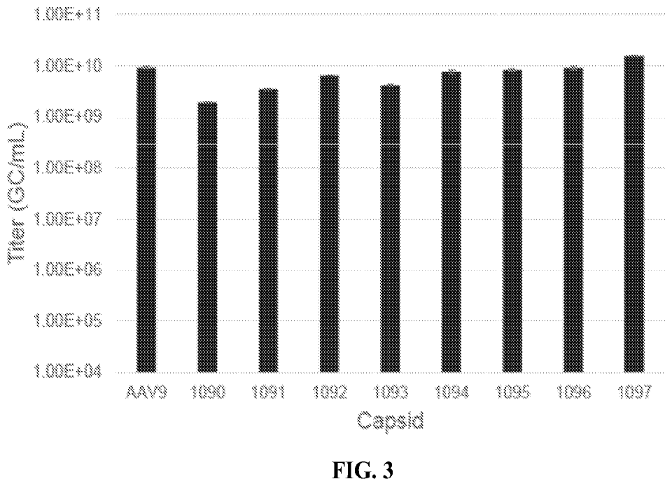

Provided are capsids with the peptide inserted at positions amenable to peptide insertions within and near the AAV9 capsid VR-IV loop (see FIG. 2) and corresponding regions on the VR-IV loop of capsids of other AAV types. Though previous studies analyzed potential positions in various AAVs, none identified the AAV9 VR-IV as amenable for this purpose (consider, e.g., Wu et al, 2000, “Mutational Analysis of the Adeno-Associated Virus Type 2 (AAV2) Capsid Gene and Construction of AAV2 Vectors with Altered Tropism,” J of Virology 74(18):8635-8647; Lochrie et al, 2006, “Adeno-associated virus (AAV) capsid genes isolated from rat and mouse liver genomic DNA define two new AAV species distantly related to AAV-5,” Virology 353:68-82; Shi and Bartlett, 2003, “RGD Inclusion in VP3 Provides Adeno-Associated Virus Type 2 (AAV2)-Based Vectors with a Heparan Sulfate-Independent Cell Entry Mechanism,” Molecular Therapy 7(4):515525-; Nicklin et al., 2001, “Efficient and Selective AAV2-Mediated Gene Transfer Directed to Human Vascular Endothelial Cells” Molecular Therapy 4(2):174-181; Grifman et al., 2001, “Incorporation of Tumor-Targeting Peptides into Recombinant Adeno-associated Virus Capsids,” Molecular Therapy 3(6):964-975; Girod et al. 1999, “Genetic capsid modifications allow efficient re-targeting of adeno-associated virus type 2,” Nature Medicine 3(9):1052-1056; Douar et al., 2003, “Deleterious effect of peptide insertions in a permissive site of the AAV2 capsid,” Virology 309:203-208; and Ponnazhagan, et al. 2001, J. of Virology 75(19):9493-9501).

Accordingly, provided are rAAV vectors carrying a RTIGPSV (SEQ ID NO:12), LGETTRP (SEQ ID NO:8) or LALGETTRPA (SEQ ID NO:9) or other peptide, for example, SEQ ID Nos: 1-20, peptide insertion at insertion points, in particular, within surface-exposed variable regions in the capsid coat, particularly within or near the variable region IV of the capsid protein. In some embodiments, the rAAV capsid protein comprises a peptide insertion immediately after (i.e., connected by a peptide bond C-terminal to) an amino acid residue corresponding to one of amino acids 451 to 461 of AAV9 capsid protein (amino acid sequence SEQ ID NO:67 and see FIG. 7 for alignment of capsid protein amino acid sequence of other AAV serotypes with amino acid sequence of the AAV9 capsid), where said peptide insertion is surface exposed when the capsid protein is packaged as an AAV particle. For example, in a particular embodiment, an AAV3B capsid protein comprises the RTIGPSV (SEQ ID NO:12) peptide insertion immediately after (i.e., connected by a peptide bond C-terminal to) an amino acid residue corresponding to one of amino acids 449 to 459 of the AAV3B (SEQ ID NO:74) or amino acids 452 to 461 of AAVrh73 capsid protein (SEQ ID NO:75), where said peptide insertion is surface exposed when the capsid protein is packaged as an AAV particle. The peptide insertions should not delete any residues of the AAV capsid protein. Generally, the peptide insertion occurs in a variable (poorly conserved) region of the capsid protein, compared with other serotypes, and in a surface exposed loop.

A peptide insertion described as inserted “at” a given site refers to insertion immediately after, that is having a peptide bond to the carboxy group of, the residue normally found at that site in the wild type virus. For example, insertion at Q588 in AAV9 means that the peptide insertion appears between Q588 and the consecutive amino acid (A589) in the AAV9 wildtype capsid protein sequence (SEQ ID NO:67). In embodiments, there is no deletion of amino acid residues at or near (within 5, 10, 15 residues or within the structural loop that is the site of the insertion) the point of insertion. In particular embodiments, the capsid protein is an AAV3B capsid protein or an AAVrh73 capsid protein and the insertion occurs immediately after at least one of the amino acid residues 449 to 459 or 451 to 461, respectively. In particular embodiments, the peptide insertion occurs immediately after amino acid residues Q449, G450, T451, T452, 5453, G454, T455, T456, N457, Q458, or S459 of the AAV3B capsid or Q452, S453, T454, G455, G456, T457, A458, G459, T460, or Q461 of the AAVrh73 capsid. In certain embodiments, the peptide is inserted between residues S454 and G455 of AAV9 capsid protein, between residues G454 and T455 of AAV3B capsid protein, between residues G457 and T458 of AAVrh73, or between the residues corresponding to S454 and G455 of an AAV capsid protein other than an AAV9 capsid protein (amino acid sequence SEQ ID NO:67). In other embodiments, the capsid protein is from at least one AAV type selected from AAV serotype 1 (AAV1), serotype 2 (AAV2), serotype 3 (AAV3), serotype 3B (AAV3B) serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype rh73 (AAVrh73), serotype hu.37 (AAVhu.37), serotype rh74 (AAVrh74, versions 1 and 2), serotype hu51 (AAV.hu51), serotype hu21 (AAV.hu21), serotype hu12 (AAV.hu12), or serotype hu26 (AAV.hu26), (see FIG. 7), and the insertion occurs immediately after an amino acid residue corresponding to at least one of the amino acid residues 451 to 461 of AAV9. The alignments of these different AAV serotypes, as shown in FIG. 7, indicates “corresponding” amino acid residues in the different capsid amino acid sequences such that a “corresponding” amino acid residue is lined up at the same position in the alignment as the residue in the reference sequence. In some particular embodiments, the peptide insertion occurs immediately after one of the amino acid residues within: 450-459 of AAV1 capsid (SEQ ID NO:59); 449-458 of AAV2 capsid (SEQ ID NO:60); 449-459 of AAV3 capsid (SEQ ID NO:61); 449-459 of AAV3B capsid (SEQ ID NO:74), 443-453 of AAV4 capsid (SEQ ID NO:62); 442-445 of AAV5 capsid (SEQ ID NO:63); 450-459 of AAV6 capsid (SEQ ID NO:64); 451-461 of AAV7 capsid (SEQ ID NO:65); 451-461 of AAV8 capsid (SEQ ID NO:66); 451-461 of AAV9 capsid (SEQ ID NO:67); 452-461 of AAV9e capsid (SEQ ID NO:68); 452-461 of AAVrh10 capsid (SEQ ID NO:69); 452-461 of AAVrh20 capsid (SEQ ID NO:70); 452-461 of AAVhu.37 (SEQ ID NO:71); 452-461 of AAVrh73; 452-461 of AAVrh74 (SEQ ID NO:72 or SEQ ID NO:96); 452-461 of AAVrh39 (SEQ ID NO:73), 449-458 of AAVhu12 (SEQ ID NO:78), 449-458 of AAVhu21 (SEQ ID NO:77), 449-458 of AAVhu26 (SEQ ID NO:79), or 449-458 of AAVhu51 (SEQ ID NO:76) in the sequences depicted in FIG. 7. In certain embodiments, the rAAV capsid protein comprises a peptide insertion immediately after (i.e., C-terminal to) amino acid 588 of AAV9 capsid protein (having the amino acid sequence of SEQ ID NO:67 and see FIG. 7), where said peptide insertion is surface exposed when the capsid protein is packaged as an AAV particle. In specific embodiments, the rAAV capsid protein comprises a peptide insertion, in particular, LALGETTRPA (SEQ ID NO:9), immediately after amino acid 588 of AAV3B capsid protein or immediately after amino acid 590 of AAVrh73 capsid protein. In other embodiments, the rAAV capsid protein has a peptide insertion that is not immediately after amino acid 588 of AAV9 or corresponding to amino acid 588 of AAV9.

In other embodiments, when the peptide is a targeting peptide, including, at least 4 contiguous amino acids, or at least 10 contiguous amino acids, or is exactly 10 contiguous amino acids, or functional fragments thereof, of RTIGPSV (SEQ ID NO:12), the capsid protein is from at least one AAV type selected from AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), serotype rh74 (AAVrh74, versions 1 and 2), serotype hu51 (AAV.hu51), serotype hu21 (AAV.hu21), serotype hu12 (AAV.hu12), or serotype hu26 (AAV.hu26) (see FIG. 7), and the peptide is inserted in the capsid protein at any point such that the peptide is surface exposed when incorporated into the AAV vector. In specific embodiments, the peptide is inserted after 138; 262-272; 450-459; or 585-593 of AAV1 capsid (SEQ ID NO:59); 138; 262-272; 449-458; or 584-592 of AAV2 capsid (SEQ ID NO:60); 138; 262-272; 449-459; or 585-593 of AAV3 capsid (SEQ ID NO:61); 138; 262-272; 449-459; or 585-593 of AAV3B capsid (SEQ ID NO:74); 137; 256-262; 443-453; or 583-591 of AAV4 capsid (SEQ ID NO:62); 137; 252-262; 442-445; or 574-582 of AAV5 capsid (SEQ ID NO:63); 138; 262-272; 450-459; 585-593 of AAV6 capsid (SEQ ID NO:64); 138; 263-273; 451-461; 586-594 of AAV7 capsid (SEQ ID NO:65); 138; 263-274; 452-461; 587-595 of AAV8 capsid (SEQ ID NO:66); 138; 262-273; 452-461; 585-593 of AAV9 capsid (SEQ ID NO:67); 138; 262-273; 452-461; 585-593 of AAV9e capsid (SEQ ID NO:68); 138; 263-274; 452-461; 587-595 of AAVrh10 capsid (SEQ ID NO:69); 138; 263-274; 452-461; 587-595 of AAVrh20 capsid (SEQ ID NO:70); 138; 263-274; 452-461; 587-595 of AAVrh73 capsid (SEQ ID NO:75); 138; 263-274; 452-461; 587-595 of AAVrh74 capsid (SEQ ID NO:72 or SEQ ID NO:96), 138; 263-274; 452-461; 587-595 of AAVhu37 capsid (SEQ ID NO:71); 138; 263-274; 452-461; 587-595 of AAVrh39 capsid (SEQ ID NO:734); 138; 264-271; 449-458; 584-592 of AAVhu12 capsid (SEQ ID NO:78); 449-458; 584-592 of AAVhu21 capsid (SEQ ID NO:77); 449-458; 584-592 of AAVhu26 capsid (SEQ ID NO:79); and 449-458; 584-592 of AAVhu51 capsid (SEQ ID NO:76) (as numbered in FIG. 7).

In some embodiments, the capsid protein is from an AAV other than serotype AAV2. In some embodiments, the peptide insertion does not occur immediately after an amino acid residue corresponding to amino acid 570 or 611 of AAV2 capsid protein. In some embodiments, the peptide insertion does not occur between amino acid residues corresponding to amino acids 587-588 of AAV2 capsid protein (see US 2014/0294771 to Schaffer et al).

Also provided are AAV vectors comprising the engineered capsids. In some embodiments, the AAV vectors are non-replicating and do not include the nucleotide sequences encoding the rep or cap proteins (these are supplied by the packaging cells in the manufacture of the rAAV vectors). In some embodiments, AAV-based vectors comprise components from one or more serotypes of AAV. In some embodiments, AAV based vectors provided herein comprise capsid components from one or more of AAV1, AAV2, AAV3, AAV3B. AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.rh73, AAV.rh74, AAV.RHM4-1, AAV.hu.26, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV3B, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.rh73, AAV.rh74, AAV.RHM4-1, AAV.hu.26, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV3B, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.rh73, AAV.rh74, AAV.RHM4-1, AAV.hu.26, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. These engineered AAV vectors may comprise a genome comprising a transgene encoding a therapeutic protein.

In particular embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the recombinant AAV for use in compositions and methods herein is AAV.7m8 (including variants thereof) (see, e.g., U.S. Pat. Nos. 9,193,956; 9,458,517; 9,587,282; US 2016/0376323, and WO 2018/075798, each of which is incorporated herein by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In certain embodiments, a single-stranded AAV (ssAAV) may be used. In certain embodiments, a self-complementary vector, e.g., scAAV, may be used (see, e.g., Wu, 2007, Human Gene Therapy, 18(2):171-82; McCarty et al, 2001, Gene Therapy, 8(16):1248-1254; U.S. Pat. Nos. 6,596,535; 7,125,717; and 7,456,683, each of which is incorporated herein by reference in its entirety).

Generally, the peptide insertion is sequence of contiguous amino acids from a heterologous protein or domain thereof. The peptide to be inserted typically is long enough to retain a particular biological function, characteristic, or feature of the protein or domain from which it is derived. The peptide to be inserted typically is short enough to allow the capsid protein to form a coat, similarly or substantially similarly to the native capsid protein without the insertion. In preferred embodiments, the peptide insertion is from about 4 to about 30 amino acid residues in length, about 4 to about 20, about 4 to about 15, about 5 to about 10, or about 7 amino acids in length. The peptide sequences for insertion are at least 4 amino acids in length and may be 5, 6, 7, 8, 9, 10, 11, 12, 13, 14, or 15 amino acids in length. In some embodiments, the peptide sequences are 16, 17, 18, 19, or 20 amino acids in length. In embodiments, the peptide is no more than 7 amino acids, 10 amino acids or 12 amino acids in length.

A “peptide insertion from a heterologous protein” in an AAV capsid protein refers to an amino acid sequence that has been introduced into the capsid protein and that is not native to any AAV serotype capsid. Non-limiting examples include a peptide of a human protein in an AAV capsid protein.

The present inventors also have surprisingly discovered particular peptides that can be used to re-target AAV vectors to specific tissues, organs, or cells; in particular, providing peptides that cause rAAV vectors to target ocular tissue. Without being bound by any one theory, a peptide, e.g., the RTIGPSV (SEQ ID NO:12) peptide, inserted in an AAV capsid variable region loop, was demonstrated to enhance transduction efficiency in ocular tissues. Such peptides can provide enhanced transport of AAV particles encapsidating a transgene across an endothelial cellular matrix.

The follow summarizes insertion sites for the peptides described herein, immediately after amino acid residues of AAV capsids as set forth below (see also, FIG. 7):
AAV1: 138; 262-272; 450-459; 595-593; and in a particular embodiment, between 453-454 (SEQ ID NO:59).
AAV2: 138; 262-272; 449-458; 584-592; and in particular embodiment, between 452-453 (SEQ ID NO:60).
AAV3: 138; 262-272; 449-459; 585-593; and in particular embodiment, between 452-453 (SEQ ID NO:61).
AAV3B: 138; 262-272; 449-459; 585-593; and in particular embodiment, between 452-453 (SEQ ID NO:74).
AAV4: 137; 256-262; 443-453; 583-591; and in particular embodiment, between 446-447 (SEQ ID NO:62).
AAV5: 137; 252-262; 442-445; 574-582; and in particular embodiment, between 445-446 (SEQ ID NO:63).
AAV6: 138; 262-272; 450-459; 585-593; and in particular embodiment, between 452-453 (SEQ ID NO:64).
AAV7: 138; 263-273; 451-461; 586-594; and in particular embodiment, between 453-454 (SEQ ID NO:65).
AAV8: 138; 263-274; 451-461; 587-595; and in particular embodiment, between 453-454 (SEQ ID NO:66).
AAV9: 138; 262-273; 452-461; 585-593; and in particular embodiment, between 454-455 (SEQ ID NO:67).
AAV9e: 138; 262-273; 452-461; 585-593; and in particular embodiment, between 454-455 (SEQ ID NO:68).
AAVrh10: 138; 263-274; 452-461; 587-595; and in particular embodiment, between 454-455 (SEQ ID NO:69).
AAVrh20: 138; 263-274; 452-461; 587-595; and in particular embodiment, between 454-455 (SEQ ID NO:70).
AAVrh39: 138; 263-274; 452-461; 587-595; and in particular embodiment, between 454-455 (SEQ ID NO:73).
AAV.hu12: 138; 263-274; 452-461; 584-595
AAV.hu21: 138; 264-271; 449-458; 584-592
AAV.hu26: 138; 264-271; 449-458; 584-592
AAV.hu51: 138; 264-271; 449-458; 584-592
AAVrh73: 138; 263-274; 452-461; 587-595; and in particular embodiment, between 456-457 (SEQ ID NO:75
AAVrh74: 138; 263-274; 452-461; 587-595; and in particular embodiment, between 454-455 (SEQ ID NO. 123 or SEQ ID NO: 144).
AAVhu.37: 138; 263-274; 452-461; 587-595; and in particular embodiment, between 454-455 (SEQ ID NO. 122)

In particular embodiments, the peptide insertion occurs between amino acid residues 588-589 of the AAV9 capsid, or between corresponding residues of another AAV type capsid as determined by an amino acid sequence alignment (for example, as in FIG. 7). In particular embodiments, the peptide insertion occurs immediately after amino acid residue 1451 to L461, S268 and Q588 of the AAV9 capsid sequence, or immediately after corresponding residues of another AAV capsid sequence (FIG. 7).

In some embodiments, one or more peptide insertions from one or more homing domains can be used in a single system. In some embodiments, the capsid is chosen and/or further modified to reduce recognition of the AAV particles by the subject's immune system, such as avoiding pre-existing antibodies in the subject. In some embodiments. In some embodiments, the capsid is chosen and/or further modified to enhance desired tropism/targeting.

### 5.3. Methods of Making rAAV Molecules

Another aspect of the present invention involves making molecules disclosed herein. In some embodiments, a molecule according to the invention is made by providing a nucleotide comprising the nucleic acid sequence encoding any of the capsid protein molecules herein; and using a packaging cell system to prepare corresponding rAAV particles with capsid coats made up of the capsid protein. In some embodiments, the nucleic acid sequence encodes a sequence having at least 60%, 70%, 80%, 85%, 90%, 91%, 92%, 93%, 94%, 95%, 96%, 97%, 98%, 99% or 99.9%, identity to the sequence of a capsid protein molecule described herein, and retains (or substantially retains) biological function of the capsid protein (including in some embodiments having an inserted peptide from a heterologous protein or domain thereof). In some embodiments, the nucleic acid encodes a sequence having at least 60%, 70%, 80%, 85%, 90%, 91%, 92%, 93%, 94%, 95%, 96%, 97%, 98%, 99% or 99.9%, identity to the sequence of the AAV9 capsid protein (SEQ ID NO:67 and see FIG. 7), while retaining (or substantially retaining) biological function of the AAV9 capsid protein. In some embodiments, the nucleic acid encodes a sequence having at least 60%, 70%, 80%, 85%, 90%, 91%, 92%, 93%, 94%, 95%, 96%, 97%, 98%, 99% or 99.9%, identity to the sequence of the AAV1 capsid protein (SEQ ID NO: 59); AAV2 capsid protein (SEQ ID NO:60); AAV3 capsid protein (SEQ ID NO:61); AAV3B capsid protein (SEQ ID NO:74); AAV4 capsid protein (SEQ ID NO:62); AAV5 capsid protein (SEQ ID NO:63); AAV6 capsid protein (SEQ ID NO:64); AAV7 capsid protein (SEQ ID NO:65); AAV8 capsid protein (SEQ ID NO:66); AAV9e capsid protein (SEQ ID NO:68); AAVrh.10 capsid protein (SEQ ID NO:69); AAVrh.20 capsid protein (SEQ ID NO:70); AAVhu.37 capsid protein (SEQ ID NO:71); AAVrh39 capsid protein (SEQ ID NO:73); AAV rh73 capsid protein (SEQ ID NO:75); AAVrh.74 capsid protein (SEQ ID NO:72 or SEQ ID NO:96); AAVhu.51 capsid protein (SEQ ID NO:76); AAVhu.21 capsid protein (SEQ ID NO:77); AAVhu.12 capsid protein (SEQ ID NO:78); AAVhu.26 capsid protein (SEQ ID NO:79); AAVrh.24 capsid protein (SEQ ID NO:87); AAVhu.38 capsid protein (SEQ ID NO:88); AAVrh.72 capsid protein (SEQ ID NO:89); AAVhu.56 capsid protein (SEQ ID NO:86); AAVcy.5 capsid protein (SEQ ID NO:90); AAVcy.6 capsid protein (SEQ ID NO:91); AAVrh.46 capsid protein (SEQ ID NO:92); AAVrh.13 capsid protein (SEQ ID NO:85); AAVrh.64.R1 capsid protein (SEQ ID NO:107); AAV9.S454-TFR3 capsid protein (SEQ ID NO: 42); AAV8.BBB capsid protein (SEQ ID NO: 26); AAV8.BBB.LD capsid protein (SEQ ID NO:27); AAV8.Y703F capsid protein (Y703F substitution in the amino acid sequence of SEQ ID NO:66, see FIG. 7 for numbering); AAV9.Y443F capsid protein (Y443F substitution in the amino acid sequence of SEQ ID NO:67, see FIG. 7 for numbering); or AAV9.Y6F capsid protein (Y6F substitution in the amino acid sequence of SEQ ID NO:66, see FIG. 7 for numbering) while retaining (or substantially retaining) biological function of the AAV9 capsid protein.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In other embodiments, the cap and rep genes are provided by a packaging cell and not present in the viral genome.

In some embodiments, the nucleic acid encoding the engineered capsid protein is cloned into an AAV Rep-Cap helper plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In embodiments, the rAAVs provided herein comprise a recombinant AAV genome that comprises an expression cassette, flanked by ITR sequences, such as AAV2 or AAV9 ITR sequences, where the expression cassette comprises a nucleotide sequence encoding a therapeutic protein for treatment of an ocular indication. In embodiments, the therapeutic protein is a VEGF fusion protein, such as aflibercept, an anti-VEGF antibody, or antigen-binding fragment thereof, such as, sevacizumab, ranibizumab, bevacizumab, or brolucizumab, an anti-kallikrein antibody, or antigen binding fragment thereof, such as lanadelumab, an anti-IL6 or anti-IL6R antibody, or antigen binding fragment thereof, such as satralizumab, sarilumab, siltuximab, clazakizumab, sirukumab, olokizumab, gerilimzumab, or tocilizumab, an anti-TNF antibody, or antigen binding fragment thereof, such as, adalimumab, infliximab, golimumab, or certolizumab-pegol, a TNF Receptor fusion protein, such as etanercept, an anti-C3 antibody, or antigen binding fragment thereof, such as, eculizumab, ravulizumab, or tesidolumab, or an anti-05 antibody, or antigen binding fragment thereof, such as NGM621.

In some embodiments, the rAAVs provide transgene delivery vectors that can be used in therapeutic and prophylactic applications, as discussed in more detail below. In some embodiments, the rAAV vector also includes regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue. In specific embodiments, the AAV vector comprises a regulatory sequence, such as a promoter, operably linked to the transgene that allows for expression in target tissues. The promoter may be a constitutive promoter, for example, the CB7 promoter. Additional promoters include: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter, or opsin promoter. In some embodiments, particularly where it may be desirable to turn off transgene expression, an inducible promoter is used, e.g., hypoxia-inducible or rapamycin-inducible promoter.

Provided in particular embodiments are AAV3B serotype, AAVrh.73 serotype, AAV.hu.26 serotype, AAVhu.51, AAVrh64R1 serotype or AAV9.S454.TFR3 capsid vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the AAV3B, AAVrh.73, AAV.hu.26, AAVhu.51, AAVrh64R1 or AAV9.S454.TFR3 capsid protein (SEQ ID NOs:74, 75, 79, 76, 107, and 42, respectively; and see FIG. 7), while retaining the biological function of the AAV3B serotype, AAVrh.73 serotype, AAV.hu.26 serotype, AAVhu.51, AAVrh64R1 serotype or AAV9.S454.TFR3 capsid. In certain embodiments, the encoded AAV3B serotype, AAVrh.73 serotype, AAV.hu.26 serotype, AAVhu.51, AAVrh64R1 serotype or AAV9.S454.TFR3 capsid has the sequence of AAV3B serotype, AAVrh.73 serotype, AAV.hu.26 serotype, AAVhu.51, AAVrh64R1 serotype or AAV9.S454.TFR3 with, in addition, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid substitutions with respect to the AAV3B serotype, AAVrh.73 serotype, AAV.hu.26 serotype, AAVhu.51, AAVrh64R1 serotype or AAV9.S454.TFR3 capsid.

The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety

The rAAV vector for delivering the transgene to target tissues, cells, or organs, has a tropism for that particular target tissue, cell, or organ, in particular the eye and tissues within the eye. Tissue-specific promoters may also be used. The construct further can include expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) control elements, which include a constitutive promoter or an ocular tissue specific promoter, optionally, an intron sequence, such as a chicken β-actin intron and a poly A signal; and (3) transgene providing (e.g., coding for) a nucleic acid or protein product of interest. In embodiments, the protein of interest is an ocular therapeutic protein, including, for example, a VEGF fusion protein, such as aflibercept, an anti-VEGF antibody, or antigen-binding fragment thereof, such as, sevacizumab, ranibizumab, bevacizumab, or brolucizumab, an anti-kallikrein antibody, or antigen binding fragment thereof, such as lanadelumab, an anti-IL6 or anti-IL6R antibody, or antigen binding fragment thereof, such as satralizumab, sarilumab, siltuximab, clazakizumab, sirukumab, olokizumab, gerilimzumab, or tocilizumab, an anti-TNF antibody, or antigen binding fragment thereof, such as, adalimumab, infliximab, golimumab, or certolizumab-pegol, a TNF Receptor fusion protein, such as etanercept, an anti-C3 antibody, or antigen binding fragment thereof, such as, eculizumab, ravulizumab, or tesidolumab, or an anti-05 antibody, or antigen binding fragment thereof, such as NGM621.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COST, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or other cell types may be used, for example, but not limited, to HuH-7, HEK293, fibrosarcoma HT-1080, HKB-11, and CAP cells. Once expressed, characteristics of the expressed product (i.e., transgene product) can be determined, including determination of the glycosylation and tyrosine sulfation patterns, using assays known in the art.

### 5.4. Therapeutic and Prophylactic Uses

Another aspect relates to therapies which involve administering a transgene via a rAAV vector according to the invention to a subject in need thereof, for delaying, preventing, treating, and/or managing an ocular disease or disorder, and/or ameliorating one or more symptoms associated therewith. A subject in need thereof includes a subject suffering from the disease or disorder, or a subject pre-disposed thereto, e.g., a subject at risk of developing or having a recurrence of the disease or disorder. Generally, a rAAV carrying a particular transgene will find use with respect to a given disease or disorder in a subject where the subject's native gene, corresponding to the transgene, is defective in providing the correct gene product, or correct amounts of the gene product. The transgene then can provide a copy of a gene that is defective in the subject.

The transgene may comprise cDNA that restores protein function to a subject having a genetic mutation(s) in the corresponding native gene. In some embodiments, the cDNA comprises associated RNA for performing genomic engineering, such as genome editing via homologous recombination. In some embodiments, the transgene encodes a therapeutic RNA, such as a shRNA, artificial miRNA, or element that influences splicing.

In embodiments, the transgene comprises a nucleotide

As described herein, the AAV vector may be selected or engineered as described herein to target the appropriate tissue or cell type, including ocular tissue, for delivery of the transgene to effect the therapeutic or prophylactic use.

In particular aspects, the rAAVs described herein find use in delivery to target ocular tissues, or target ocular tissue cell types, including cell matrix associated with the target cell types, associated with the disorder or disease to be treated/prevented. A disease or disorder associated with a particular tissue or cell type is one that largely affects the particular tissue or cell type, in comparison to other tissue of cell types of the body, or one where the effects or symptoms of the disorder appear in the particular tissue or cell type. Methods of delivering a transgene to a target tissue of a subject in need thereof involve administering to the subject an rAAV where the capsid has a tropism for the tissue cell type, including enhanced transduction, genome integration, transgene mRNA and protein expression in ocular tissue, including as compared to an rAAV having a reference capsid, such as AAV2, AAV8 or AAV9.

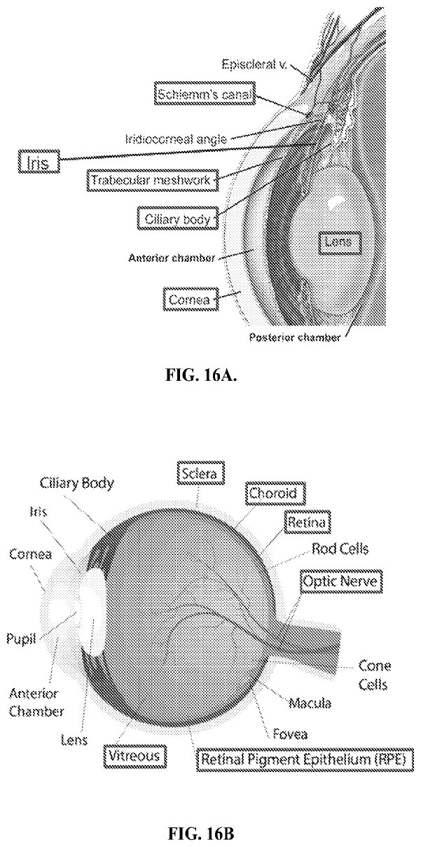

For a disease or disorder associated with the retina or eye, the rAAV vector has a capsid with ocular tropism, directing the rAAV to target the eye or ocular tissues of the subject, including, in embodiments, crossing the blood-eye barrier. The term “retinal cell” refers to one or more of the cell types found in or near the retina, including amacrine cells, bipolar cells, horizontal cells, Muller glial cells, photoreceptor cells (e.g., rods and cones), retinal ganglion cells (e.g., midget cells, parasol cells, bistratified cells, giant retina ganglion cells, and photosensitive ganglion cells), retinal pigmented epithelium, endothelial cells of the inner limiting membrane, and the like. Ocular tissues include anterior segment tissues, including the iris, cornea, lens, ciliary body, Schlemm's canal, and trabecular meshwork, and posterior segment tissues, such as the retina or RPE-choroid, and optic nerve (see FIGS. 16A and 16B).

In additional embodiments, methods and compositions are provided in which an rAAV comprising a recombinant genome comprising a transgene encoding an ocular therapeutic have a capsid with a tropism for transduction and/or transgene expression in ocular tissue, including anterior and/or posterior segments, with a capsid of an AAV serotype 1 (AAV1; SEQ ID NO: 59); AAV serotype 2 (AAV2; SEQ ID NO:60); AAV serotype 3 (AAV3; SEQ ID NO:61), AAV serotype 3B (AAV3B; SEQ ID NO:74), AAV serotype 4 (AAV4; SEQ ID NO:62); AAV serotype 5 (AAV5; SEQ ID NO:63); AAV serotype 6 (AAV6; SEQ ID NO:64); AAV serotype 7 (AAV7; SEQ ID NO:65); AAV serotype 8 (AAV8; SEQ ID NO:66); AAV serotype 9 (AAV9; SEQ ID NO:67); AAV serotype 9e (AAV9e; SEQ ID NO:68); AAV serotype rh.10 (AAVrh.10; SEQ ID NO:69); AAV serotype rh.20 (AAV.rh.20; SEQ ID NO:70); AAV serotype hu.37 (AAVhu.37; SEQ ID NO:71), AAV serotype rh39 (AAVrh.39; SEQ ID NO:73), AAV serotype rh73 (AAVrh.73; SEQ ID NO:75), AAV serotype rh.74 (AAVrh.74; SEQ ID NO:72 or SEQ ID NO:96), AAV serotype hu51 (AAVhu.51; SEQ ID NO:76), AAV serotype hu.21 (AAVhu.21; SEQ ID NO:77), AAV serotype hu.12 (AAVhu.12; SEQ ID NO:78), AAV serotype hu.26 (AAVhu.26; SEQ ID NO:79), AAV serotype rh.24 (AAVrh.24; SEQ ID NO:87), AAV serotype hu.38 (AAVhu.38; SEQ ID NO:88), AAV serotype rh.72 (AAVrh.72; SEQ ID NO:89), AAV serotype hu.56 (AAVhu.56; SEQ ID NO:86), AAV serotype cy.5 (AAVcy.5; SEQ ID NO:90), AAV serotype cy.6 (AAVcy.6; SEQ ID NO:91), AAV serotype rh.46 (AAVrh.46; SEQ ID NO:92), AAV serotype rh.13 (AAV.rh.13; SEQ ID NO:85), or AAV serotype rh.64.R1 (AAVrh.64.R1; SEQ ID NO:107), or the capsid is an engineered capsid having an insertion and/or one or more amino acid substitutions relative to one of the capsids disclosed herein, including, AAV9.5454-TFR3 (SEQ ID NO: 42), AAV8.BBB (A269S substitution (SEQ ID NO: 26)), AAV8.BBB.LD (A296S, 498 NNN/AAA 500; SEQ ID NO:27)), AAV8.Y703F (Y703F substitution in the amino acid sequence of SEQ ID NO:66, see FIG. 7 for numbering), AAV9.Y443F (Y443F substitution in the amino acid sequence of SEQ ID NO:67, see FIG. 7 for numbering), AAV9.Y6F (Y6F substitution in the amino acid sequence of SEQ ID NO:66, see FIG. 7 for numbering) (see FIG. 7 or Table 10). In certain embodiments, the rAAV has a capsid of an AAV3B serotype, AAVrh.73 serotype, AAV.hu.26 serotype, AAVhu.51, AAVrh64R1 serotype or is AAV9.S454.TFR3. In specific embodiments the rAAV is administered in the absence of hyaluronic acid, including where the rAAV has not been previously incubated with or admixed with hyaluronic acid (including hyaluronic acid that is at a concentration 0.1%, 0.25%, 0.3%, 0.35%, 0.4%, 0.45%, 0.5%, 0.6%, 0.75%, or 1.0% weight by volume).

Generally, where the rAAV vector has a tropism for ocular tissues, the vector is administered by in vivo injection, such as injection directly into the eye. For example, the rAAV comprising a peptide insertion for increasing tropism for ocular, retinal or RPE-choroid tissue may be injected intravitreally, intracamerally or suprachoroidally. In some embodiments, the rAAV with ocular tissue tropism is administered by intraocular injection, e.g., through the pars plana into the vitreous body or aqueous humor of the eye. In some embodiments, the rAAV for increasing ocular tissue tropism is administered peribulbar injection or subconjunctival injection. In some embodiments, the rAAV with ocular tissue tropism is administered by suprachoroidal injection, that is in the space between the sclera and the choroid. One advantage of rAAV vectors with ocular tissue tropism, is that the subject may avoid surgery, e.g., avoiding surgery to implant the therapeutic instead delivered by injection. In certain embodiments, the therapeutic is delivered by a rAAV vector described herein by intracameral, intravitreal or suprachoroidal injection, to provide a therapeutically effective amount for treating a disease or disorder associated with the eye, particularly, a disease or disorder associated with the eye of the subject. In more embodiments, treatment is achieved following a single intracameral, intravitreal or suprachoroidal injection, not more than two intracameral, intravitreal or suprachoroidal injections, not more than three intracameral, intravitreal or suprachoroidal injections, not more than four intracameral, intravitreal or suprachoroidal injections, not more than five intracameral, intravitreal or suprachoroidal injections, or not more than six intracameral, intravitreal or suprachoroidal injections.

Diseases/disorders associated with the eye or retina are referred to as “ocular diseases.” Nonlimiting examples of ocular diseases include anterior ischemic optic neuropathy; acute macular neuroretinopathy; Bardet-Biedl syndrome; Behcet's disease; branch retinal vein occlusion; central retinal vein occlusion; choroideremia; choroidal neovascularization; chorioretinal degeneration; cone-rod dystrophy; color vision disorders (e.g., achromatopsia, protanopia, deuteranopia, and tritanopia); congenital stationary night blindness; diabetic uveitis; epiretinal membrane disorders; inherited macular degeneration; histoplasmosis; macular degeneration (e.g., acute macular degeneration, non-exudative age related macular degeneration, exudative age related macular degeneration); diabetic retinopathy; edema (e.g., macular edema, cystoid macular edema, diabetic macular edema); glaucoma; Leber congenital amaurosis; Leber's hereditary optic neuropathy; macular telangiectasia; multifocal choroiditis; non-retinopathy diabetic retinal dysfunction; ocular trauma; ocular tumors; proliferative vitreoretinopathy (PVR); retinopathy of prematurity; retinoschisis; retinitis pigmentosa; retinal arterial occlusive disease, retinal detachment, Stargardt disease (fundus flavimaculatus); sympathetic opthalmia; uveal diffusion; uveitic retinal disease; Usher syndrome; Vogt Koyanagi-Harada (VKH) syndrome; or a posterior ocular condition associated with ocular laser or photodynamic therapy.

In particular embodiments, the disease or disorder is non-infectious uveitis, neuromyelitis optica, macular degeneration, including dry age-related macular degeration, macular edema, diabetic retinopathy or glaucoma.

In particular embodiments, the rAAV targets (including, transduction and transgene expression) one or more specific ocular tissues, including the anterior segment tissues or the posterior segment tissues and, in more specific embodiments, the rAAV targets the cornea, iris or lens, or ciliary body, Schlemm's canal or trabecular meshwork, or retinal, retinal pigment epithelium (RPE-) choroid or sclera, or the optic nerve. In particular embodiments, rAAVs having an AAV3B or AAVrh.73 capsid may be administered to target the iris, retina, RPE choroid or sclera, and in certain embodiments, the ciliary body, Schlemm's canal, trabelcular meshwork or optic nerve (orbital and/or cranial segment). In embodiments, rAAVs having an AAV3B or AAVrh.73 capsid may be used to target the retina and/or RPE choroid tissue. In other embodiments, rAAVs having an AAVrh.73 capsid may be used to target the iris tissue, and in other embodiments, AAVhu.26 capsids may be used to target the ciliary body or the trabecular meshwork. AAV1 capsids may be used to target the trabecular meshwork or the sclera and AAV7 may be used to target the trabecular meshwork.

In certain embodiments, the transgene comprises a nucleotide sequence which encodes an ocular disease therapeutic which is a VEGF fusion protein, such as aflibercept, an anti-VEGF antibody, or antigen-binding fragment thereof, such as, sevacizumab, ranibizumab, bevacizumab, or brolucizumab, an anti-kallikrein antibody, or antigen binding fragment thereof, such as lanadelumab, an anti-IL6 or anti-IL6R antibody, or antigen binding fragment thereof, such as satralizumab, sarilumab, siltuximab, clazakizumab, sirukumab, olokizumab, gerilimzumab, or tocilizumab, an anti-TNF antibody, or antigen binding fragment thereof, such as, adalimumab, infliximab, golimumab, or certolizumab-pegol, a TNF Receptor fusion protein, such as etanercept, an anti-C3 antibody, or antigen binding fragment thereof, such as, eculizumab, ravulizumab, or tesidolumab, or an anti-05 antibody, or antigen binding fragment thereof, such as NGM621, or LKA-651, solanezumab, GSK933776, lecanemab, ascrinvacumab, carotuximab, AND-007, or inebilizumab. Gene therapy constructs encoding antibodies, or antigen binding fragments thereof, are designed such that both the heavy and light chains are expressed. The coding sequences for the heavy and light chains can be engineered in a single construct in which the heavy and light chains are separated by a cleavable linker or IRES, such as a furin-T2A linker or the like, so that separate heavy and light chain polypeptides are expressed. In certain embodiments, the coding sequences encode for a Fab or F(ab′)₂ or an scFv. In certain embodiments the full length heavy and light chains of the antibody are expressed. In other embodiments, the constructs express an scFv in which the heavy and light chain variable domains are connected via a flexible, non-cleavable linker. The nucleotide sequence coding for the therapeutic protein is operably linked to regulatory elements to promote expression of the therapeutic protein in the target ocular tissue.

The rAAV vectors of the invention also can facilitate delivery, in particular, targeted delivery, of oligonucleotides, drugs, imaging agents, inorganic nanoparticles, liposomes, antibodies to target cells or tissues. The rAAV vectors also can facilitate delivery, in particular, targeted delivery, of non-coding DNA, RNA, or oligonucleotides to target tissues.

The agents may be provided as pharmaceutically acceptable compositions as known in the art and/or as described herein. Also, the rAAV molecule of the invention may be administered alone or in combination with other prophylactic and/or therapeutic agents.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some embodiments, animal model systems for a ocular condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

Toxicity and efficacy of the prophylactic and/or therapeutic agents of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

A rAAV generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes, or about 1×10¹⁰ to about 1×10¹⁵ genomes, about 1×10¹² to about 1×10¹⁶ genomes, about 1×10¹⁴ to about 1×10¹⁶ genomes, about 1×10¹¹ to about 1×10¹³ genomes, or about 1×10¹² to about 1×10¹⁴ genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once or may be administered 2, 3 or 4 times, for example, separated by a week, month, 2 months or three months.

The rAAV molecules of the invention may be administered alone or in combination with other prophylactic and/or therapeutic agents. Each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route.

In various embodiments, the different prophylactic and/or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart, or no more than 48 hours apart. In certain embodiments, two or more agents are administered within the same patient visit.

Methods of administering agents described herein include, but are not limited to, parenteral administration (e.g., intradermal, intramuscular, intraperitoneal, intravenous, and subcutaneous, including infusion or bolus injection), epidural, and by absorption through epithelial or mucocutaneous or mucosal linings (e.g., intranasal, oral mucosa, rectal, and intestinal mucosa, etc.). In particular embodiments, such as where the transgene is intended to be expressed in the eye, the vector is administered via intravitreal, intraocular, suprachoroidal, or intracameral injection. In particular embodiments, the vector is administered directly to the target tissue, for example, is administered directly to the retina or ciliary body.

In certain embodiments, the agents of the invention are administered intravenously and may be administered together with other biologically active agents.

In another specific embodiment, agents of the invention may be delivered in a sustained release formulation, e.g., where the formulations provide extended release and thus extended half-life of the administered agent. Controlled release systems suitable for use include, without limitation, diffusion-controlled, solvent-controlled, and chemically-controlled systems. Diffusion controlled systems include, for example reservoir devices, in which the molecules of the invention are enclosed within a device such that release of the molecules is controlled by permeation through a diffusion barrier. Common reservoir devices include, for example, membranes, capsules, microcapsules, liposomes, and hollow fibers. Monolithic (matrix) device are a second type of diffusion controlled system, wherein the dual antigen-binding molecules are dispersed or dissolved in an rate-controlling matrix (e.g., a polymer matrix). Agents of the invention can be homogeneously dispersed throughout a rate-controlling matrix and the rate of release is controlled by diffusion through the matrix. Polymers suitable for use in the monolithic matrix device include naturally occurring polymers, synthetic polymers and synthetically modified natural polymers, as well as polymer derivatives.

Any technique known to one of skill in the art can be used to produce sustained release formulations comprising one or more agents described herein. See, e.g. U.S. Pat. No. 4,526,938; PCT publication WO 91/05548; PCT publication WO 96/20698; Ning et al., “Intratumoral Radioimmunotheraphy of a Human Colon Cancer Xenograft Using a Sustained-Release Gel,” Radiotherapy & Oncology, 39:179 189, 1996; Song et al., “Antibody Mediated Lung Targeting of Long-Circulating Emulsions,” PDA Journal of Pharmaceutical Science & Technology, 50:372 397, 1995; Cleek et al., “Biodegradable Polymeric Carriers for a bFGF Antibody for Cardiovascular Application,” Pro. Intl. Symp. Control. Rel. Bioact. Mater., 24:853 854, 1997; and Lam et al., “Microencapsulation of Recombinant Humanized Monoclonal Antibody for Local Delivery,” Proc. Int'l. Symp. Control Rel. Bioact. Mater., 24:759 760, 1997, each of which is incorporated herein by reference in its entirety. In one embodiment, a pump may be used in a controlled release system (see Langer, supra; Sefton, CRC Crit. Ref. Biomed. Eng., 14:20, 1987; Buchwald et al., Surgery, 88:507, 1980; and Saudek et al., N. Engl. J. Med., 321:574, 1989). In another embodiment, polymeric materials can be used to achieve controlled release of agents comprising dual antigen-binding molecule, or antigen-binding fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., Macromol. Sci. Rev. Macromol. Chem, 23:61, 1983; see also Levy et al., Science, 228:190, 1985; During et al., Ann. Neurol., 25:351, 1989; Howard et al., J. Neurosurg., 7 1:105, 1989); U.S. Pat. Nos. 5,679,377; 5,916,597; 5,912,015; 5,989,463; 5,128,326; PCT Publication No. WO 99/15154; and PCT Publication No. WO 99/20253). In yet another embodiment, a controlled release system can be placed in proximity of the therapeutic target (e.g., an affected joint), thus requiring only a fraction of the systemic dose (see, e.g., Goodson, in Medical Applications of Controlled Release, supra, vol. 2, pp. 115 138 (1984)). Other controlled release systems are discussed in the review by Langer, Science, 249:1527 1533, 1990.

In addition, rAAVs can be used for in vivo delivery of transgenes for scientific studies such as optogenetics, gene knock-down with miRNAs, recombinase delivery for conditional gene deletion, gene editing with CRISPRs, and the like.

### 5.5. Pharmaceutical Compositions and Kits

The invention further provides a pharmaceutical composition comprising a pharmaceutically acceptable carrier and an agent of the invention, said agent comprising a rAAV molecule of the invention. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In certain embodiments, the agent of the invention is substantially purified (i.e., substantially free from substances that limit its effect or produce undesired side-effects). In a specific embodiment, the host or subject is an animal, e.g., a mammal such as non-primate (e.g., cows, pigs, horses, cats, dogs, rats etc.) and a primate (e.g., monkey such as, a cynomolgus monkey and a human). In a certain embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The compositions of the invention include bulk drug compositions useful in the manufacture of pharmaceutical compositions (e.g., impure or non-sterile compositions) as well as pharmaceutical compositions (i.e., compositions that are suitable for administration to a subject or patient). Bulk drug compositions can be used in the preparation of unit dosage forms, e.g., comprising a prophylactically or therapeutically effective amount of an agent disclosed herein or a combination of those agents and a pharmaceutically acceptable carrier.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

The following examples report an analysis of surface-exposed loops on the AAV9 capsid to identify candidates for capsid engineering via insertional mutagenesis. Further examples, demonstrate the increased transduction and tissue tropism for various AAV capsids described herein.

### 6.1. Example 1—Analysis of AAV9 Capsid

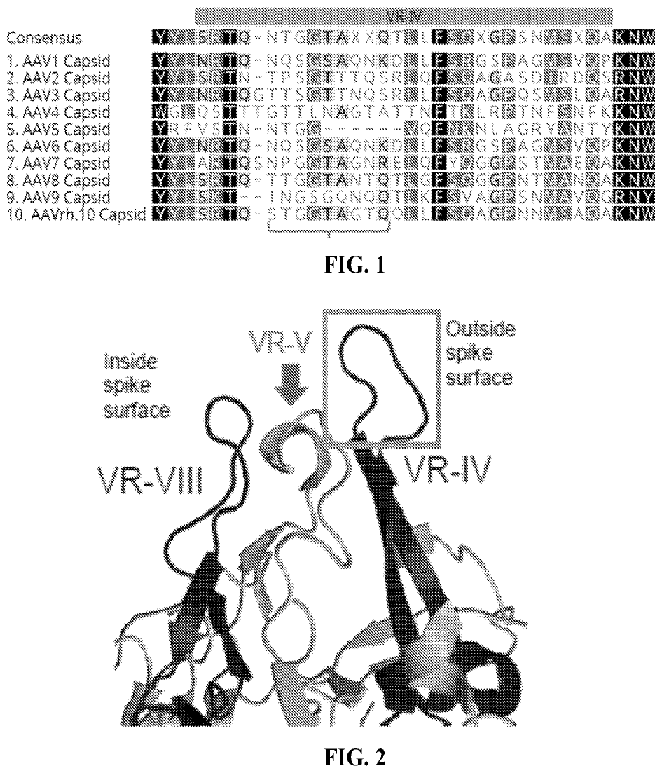

FIGS. 1 and 2 depict analysis of variable region four of the adeno-associated virus type 9 (AAV9 VR-IV) by amino acid sequence comparison to other AAVs VR-IV (FIG. 1) and protein model (FIG. 2). As seen, AAV9 VR-IV is exposed on the surface at the tip or outer surface of the 3-fold spike. Further analysis indicated that there are few side chain interactions between VR-IV and VR-V and that the sequence and structure of VR-IV is variable amongst AAV serotypes, and further that there is potential for interrupting a commonly-targeted neutralizing antibody epitope and thus, reducing immunogenicity of the modified capsid.

### 6.2. Example 2—Construction of AAV9 Mutants

Eight AAV9 mutants were constructed, to each include a heterologous peptide but at different insertion points in the VR-IV loop. The heterologous peptide was a FLAG tag that was inserted immediately following the following residues in vectors identified as pRGNX1090-1097, as shown in Table 1.

TABLE 1

| Vector designation | AAV9 VR-IV Insertion site for FLAG tag |
|---|---|
| pRGNX1090 | I451 |
| pRGNX1091 | N452 |
| pRGNX1092 | G453 |
| pRGNX1093 | S454 |
| pRGNX1094 | G455 |
| pRGNX1095 | Q456 |
| pRGNX1096 | N457 |
| pRGNX1097 | Q458 |

### 6.3. Example 3—Analysis of Packaging Efficiency

FIG. 3 depicts high packaging efficiency in terms of genome copies per mL (GC/mL) of wild type AAV9 and eight (8) candidate rAAV9 vectors (1090, 1091, 1092, 1093, 1094, 1095, 1096, and 1097), where the candidate vectors each contain a FLAG insert at different sites within AAV9's VR-IV. All vectors were packaged with luciferase transgene in 10 mL culture to facilitate determining which insertion points did not interrupt capsid packaging; error bars represent standard error of the mean.

As seen, all candidates package with high efficiency.

### 6.4. Example 4—Analysis of Surface FLAG Exposure

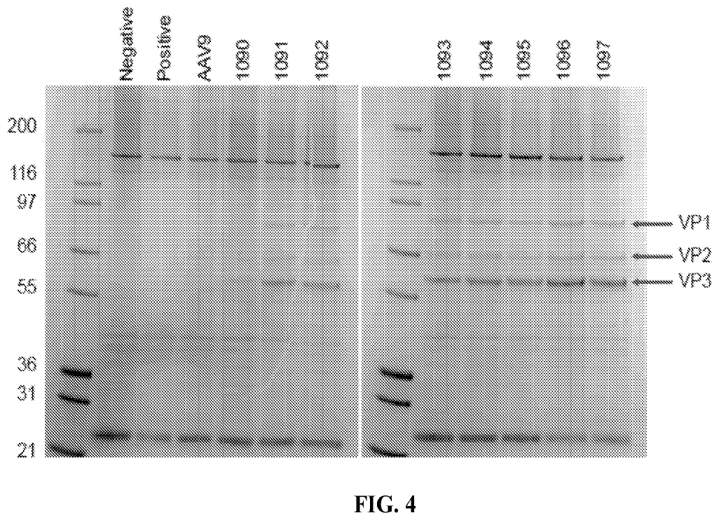

FIG. 4 depicts surface exposure of FLAG inserts in each of eight (8) candidate rAAV9 vectors (1090, 1091, 1092, 1093, 1094, 1095, 1096, and 1097), confirmed by immunoprecipitation of transduced vectors by binding to anti-FLAG resin. Binding to anti-FLAG indicates insertion points that allow formation of capsids that display the peptide insertion on the surface.

Transduced cells were lysed and centrifuged. 500 μL of cell culture supernatant was loaded on 20 μL agarose-FLAG beads and eluted with SDS-PAGE loading buffer also loaded directly on the gel. For a negative control, 293-ssc supernatant was used that contained no FLAG inserts.

As seen, 1090 had the lowest titer of the candidate vectors, indicating the least protein pulled down. Very low titers also were seen with the positive control. It is likely that not a sufficient amount of positive control had been loaded for visualization on SDS-PAGE.

### 6.5. Example 5—Analysis of Transduction Efficiency

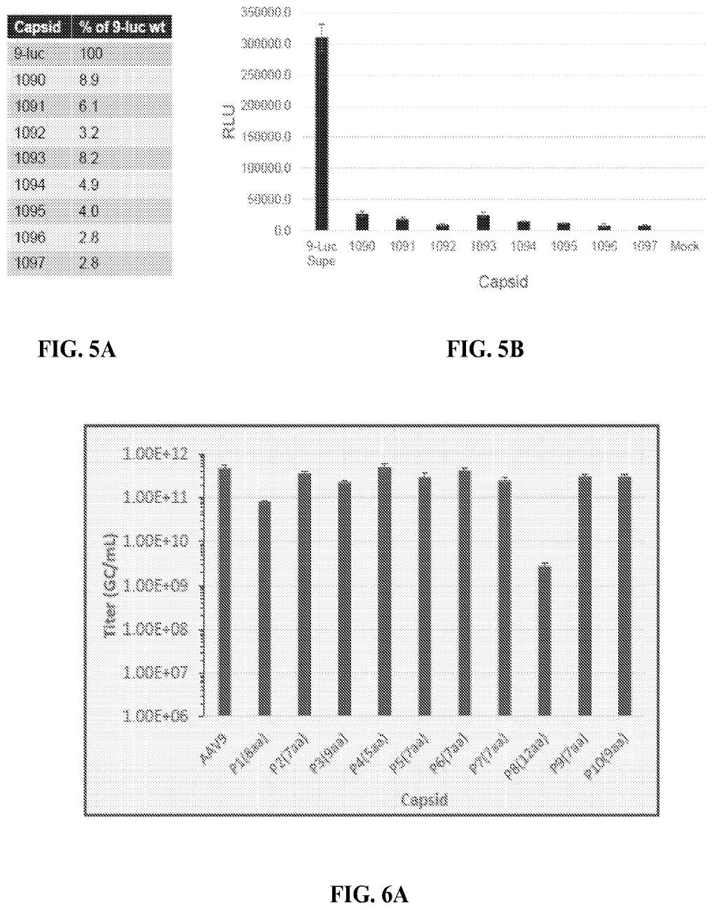

FIGS. 5A-5B depict transduction efficiency in Lec2 cells, transduced with capsid vectors carrying the luciferase gene as a transgene, that was packaged into either wild type AAV9 (9-luc), or into each of eight (8) candidate rAAV9 vectors (1090, 1091, 1092, 1093, 1094, 1095, 1096, and 1097); activity is expressed as percent luciferase activity, taking the activity of 9-luc as 100% (FIG. 5A), or as Relative Light Units (RLU) per microgram of protein (FIG. 5B).

CHO-derived Lec2 cells were grown in αMEM and 10% FBS. The Lec2 cells were transduced at a MOI of about 2×10⁸ GC vector (a MOI of about 10,000) and were treated with ViraDuctin reagent (similar results were observed on transducing Lec2 cells at a MOI of about GC/cell but treated with 40 μg/mL zinc chloride (ZnCl₂); results not shown). Lec2 cells are proline auxotrophs from CHO.

As seen, transduction efficiency in vitro is lower than that obtained using wild type AAV9 (9-luc). Nonetheless, previous studies have shown that introduction of a homing peptide can decrease in vitro gene transfer in non-target cells (such as 293, Lec2, or HeLa), while significantly increasing in vitro gene transfer in target cells (see, e.g., Nicklin et al. 2001; and Grifman et al. 2001).

### 6.6. Example 6—Analysis of Packaging Efficiency as a Factor of Insertion Peptide Composition and Length

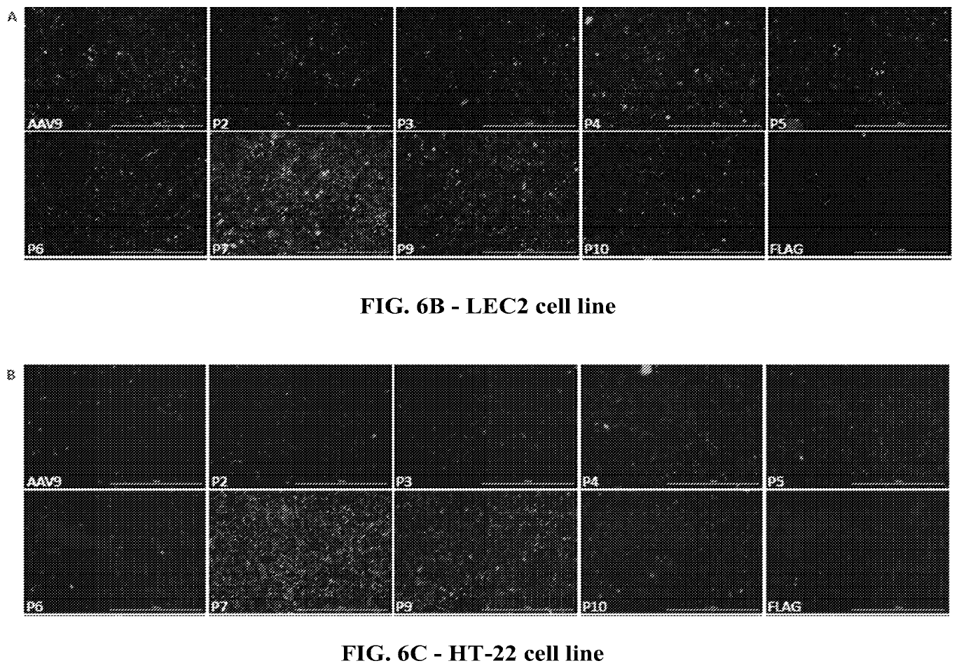

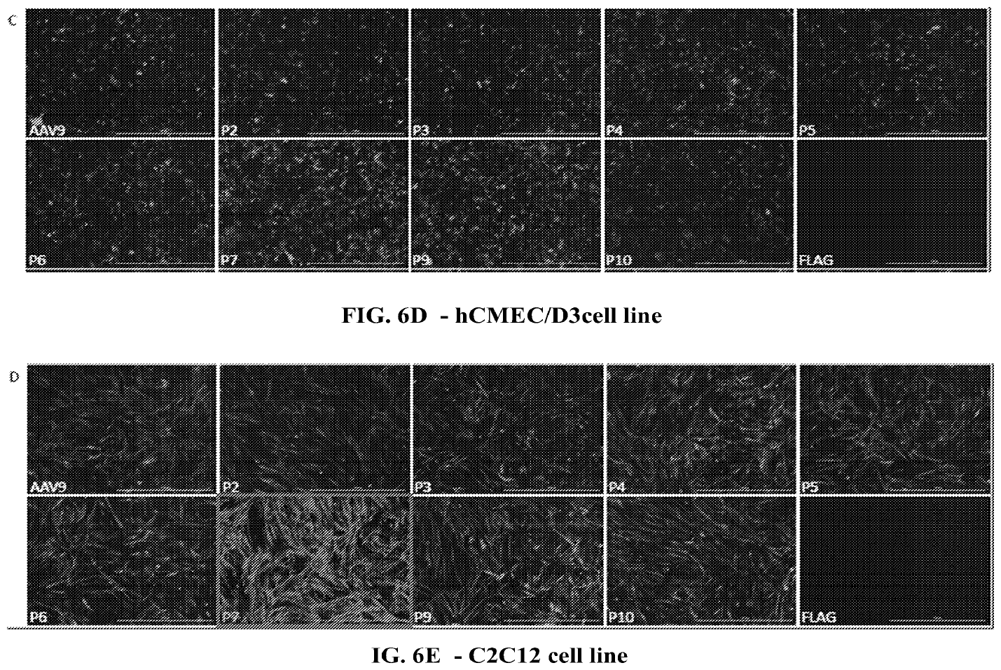

FIG. 6A depicts a bar graph illustrating that insertions immediately after S454 of AAV9 capsid (SEQ ID NO:67) of varying peptide length and composition may affect production efficiencies of AAV particles in a packaging cell line. Ten peptides of varying composition and length were inserted after S454 (between residues 454 and 455) within AAV9 VR-IV. qPCR was performed on harvested supernatant of transfected suspension HEK293 cells five days post-transfection. The results depicted in the bar graph demonstrate that the nature and length of the insertions may affect the ability of AAV particles to be produced at high titer and packaged in 293 cells. (Error bars represent standard error of the mean length of peptide, which is noted on the Y-axis in parenthesis.)

AAV9 vectors having an capsid protein containing a homing peptide of the following peptide sequences (Table 2) at the S454 insertion site were studied. Suspension-adapted HEK293 cells were seeded at 1×10⁶ cells/mL one day before transduction in 10 mL of media Triple plasmid DNA transfections were done with PEIpro® (Polypus transfection) at a DNA:PEI ratio of 1:1.75. Cells were spun down and supernatant harvested five days post-transfection and stored at −80° C.

TABLE 2

| Peptide | Tissue or Target | Peptide | SEQ ID |
|---|---|---|---|
| # | Designation | Sequence | NO: |
| P1 | Bone1 (D8) | DDDDDDDD | 2 |
| P2 | Brain1 | LSSRLDA | 3 |
| P3 | Brain2 | CLSSRLDAC | 4 |
| P4 | Kidney1 | LPVAS | 6 |
| P5 | Kidney2 | CLPVASC | 5 |
| P6 | Muscle1 | ASSLNIA | 7 |
| P7 | TfR1 | HAIYPRH | 10 |
| P8 | TfR2 | THRPPMWSPVWP | 11 |
| P9 | TfR3 | RTIGPSV | 12 |
| P10 | TfR4 | CRTIGPSVC | 13 |

qPCR was performed on harvested supernatant of transfected suspension HEK293 cells five days post-transfection. Samples were subjected to DNase I treatment to remove residual plasmid or cellular DNA and then heat treated to inactivate DNase I and denature capsids. Samples were titered via qPCR using TaqMan Universal PCR Master Mix, No AmpEraseUNG (ThermoFisherScientific) and primer/probe against the polyA sequence packaged in the transgene construct. Standard curves were established using RGX-501 vector BDS.

Peptide insertions directly after 5454 ranging from 5 to 10 amino acids in length produced AAV particles having adequate titer, whereas an upper size limit is possible, with significant packaging deficiencies observed for the peptide insertion having a length of 12 amino acids.

### 6.7. Example 7—Homing Peptides Alter the Transduction Properties of AAV9 In Vitro when Inserted after S454

FIGS. 6B-E depict fluorescence images of cell cultures of (FIG. 6B) Lec2 cell line (sialic acid-deficient epithelial cell line) (FIG. 6C) HT-22 cell line (neuronal cell line), (FIG. 6D) hCMEC/D3 cell line (brain endothelial cell line), and (FIG. 6E) C2C12 cell line (muscle cell line). AAV9 wild type and S454 insertion homing peptide capsids of Table 2 containing GFP transgene were used to transduce the noted cell lines.

Cell lines were plated at 5-20×10³ cells/well (depending on the cell line) in 96-well 24 hours before transduction. Cells were transduced with AAV9-GFP vectors (with or without insertions) at 1×10¹⁰ particles/well and analyzed via Cytation5 (BioTek) 48-96 hours after transduction, depending on the difference in expression rate in each cell line. Lec2 cells were cultured as in Example 5, blood-brain barrier hCMEC/D3 (EMD Millipore) cells were cultured according to manufacturer's protocol, HT-22 and HUH7 cells were cultured in DMEM and 10% FBS, and C2C12 myoblasts were plated in DMEM and 10% FBS and differentiated for three days pre-transfection in DMEM supplemented with 2% horse serum and 0.1% insulin. AAV9.S454.FLAG showed low transduction levels in every cell type tested.

Images show that homing peptides can alter the transduction properties of AAV9 in vitro when inserted after S454 in the AAV9 capsid protein, as compared to unmodified AAV9 capsid. P7 (TfR1 peptide, HAIYPRH (SEQ ID NO:10)) for all cell lines show the highest rate of transduction followed by P9 (TfR3 peptide, RTIGPSV (SEQ ID NO:12)). P4 (Kidney 1 peptide, LPVAS (SEQ ID NO:6)) showed a slightly higher rate of transduction than that of AAV9 wildtype for all cell types. Higher transduction rates were observed for P6 (Muscle1 peptide, ASSLNIA (SEQ ID NO:7)) in the brain endothelial hCMEC/D3 cell line and the C2C12 muscle cell line cultures as compared to the Lec2 and HT-22 cell line cultures. P1 vector was not included in images due to extremely low transduction efficiency, and P8 vector was not included due to low titer.

### 6.8. Example 8—Analysis of AAV Capsids for Peptide Insertion Points

FIG. 7 depicts alignment of AAVs 1-9e, 3B, rh10, rh20, rh39, rh73, rh74 version 1 and version 2, hu12, hu21, hu26, hu37, hu51 and hu53 sequences within insertion sites for peptides that enhance ocular tissue tropism within or near the initiation codon of VP2, variable region 1 (VR-I), variable region 4 (VR-IV), and variable region 8 (VR-VIII) highlighted in grey; a particular insertion site within variable region eight (VR-VIII) of each capsid protein is shown by the symbol “4” (after amino acid residue 588 according to the amino acid numbering of AAV9).

### 6.9. Example 9—Comparison of AAV Genome Copies/μg Genomic DNA of Various Vectors

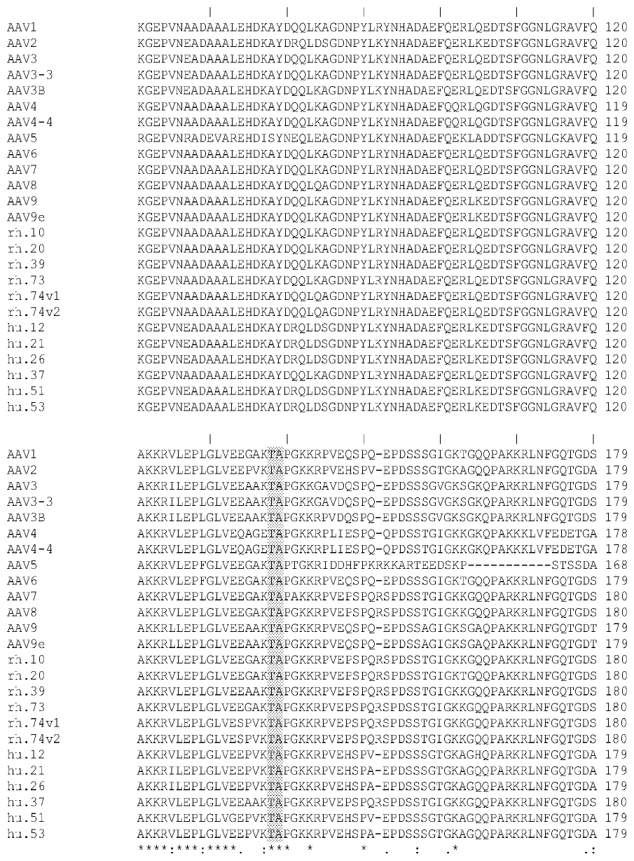

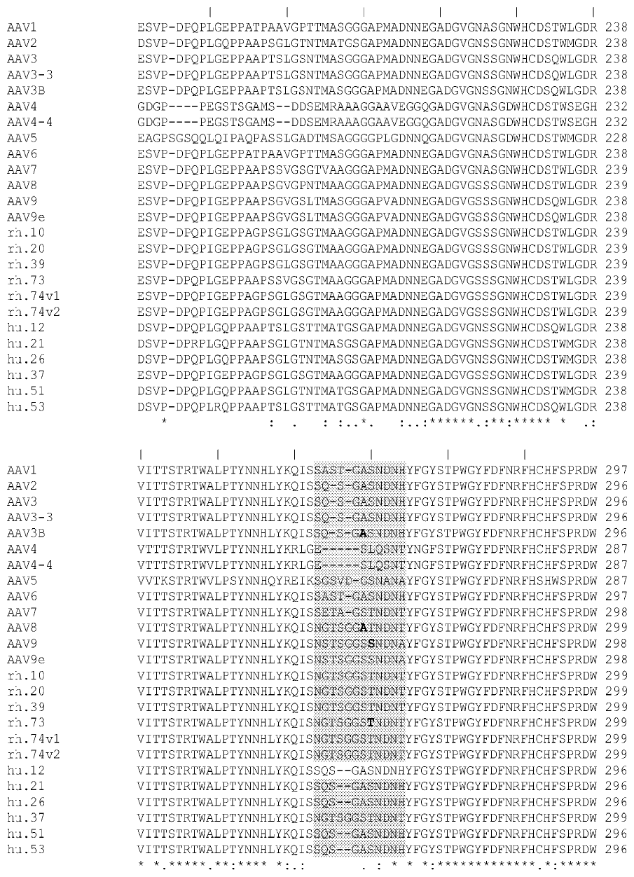

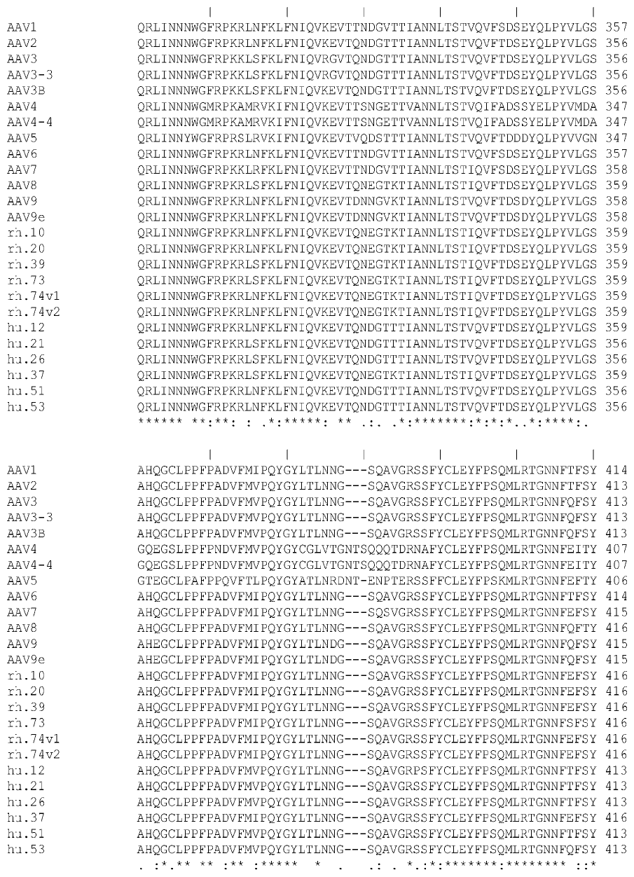

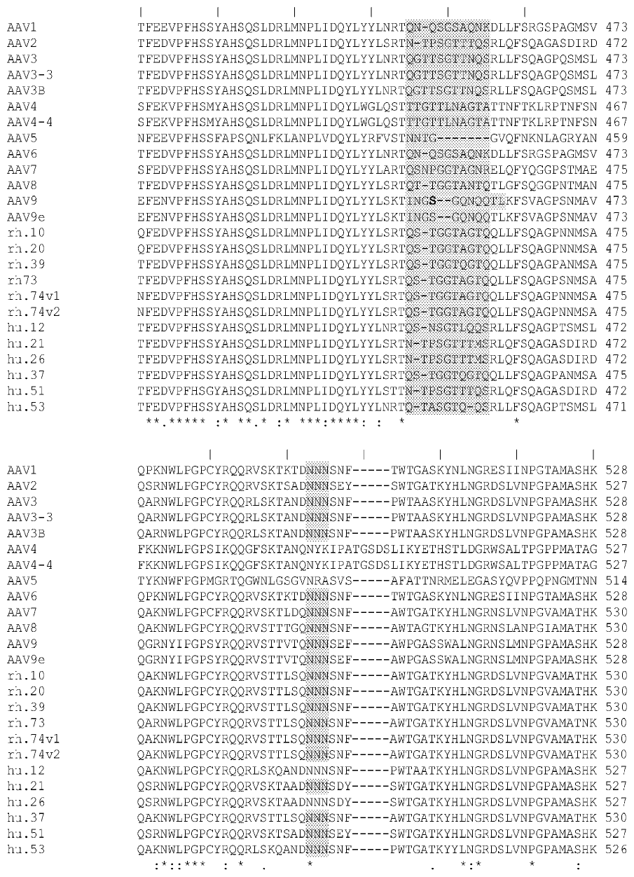

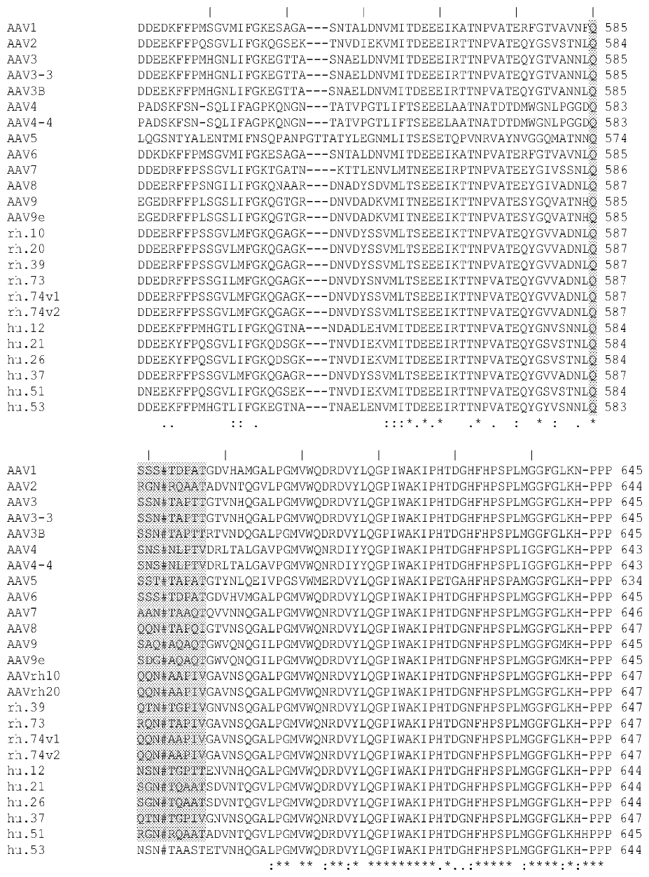

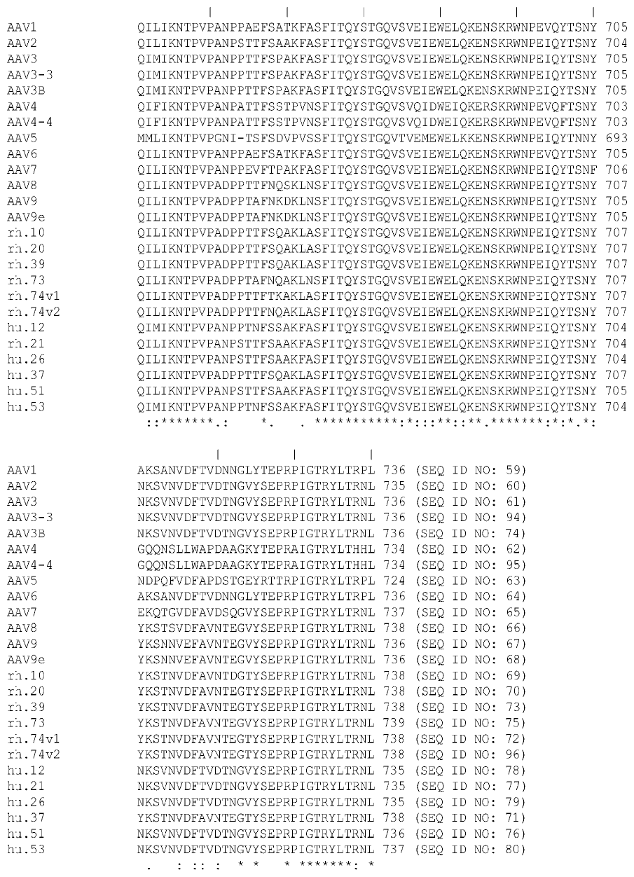

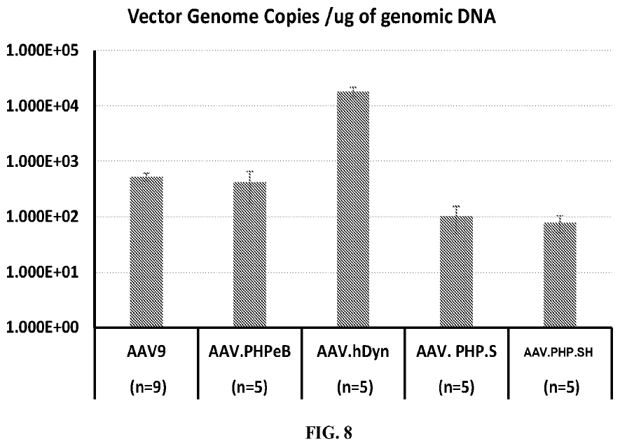

FIG. 8 depicts copies of GFA (green fluorescent protein) transgene expressed in mouse brain cells, following administration of the AAV vectors: AAV9; AAV.PHP.eB; AAV.hDyn (AAV9 with TLAAPFK (SEQ ID NO:1) between 588-589 with no other amino acid modifications to the capsid sequence); AAV.PHP.S; and AAV.PHP.SH (see Table 10).

AAV.PHP.B is a capsid having a TLAVPFK (SEQ ID NO:20) insertion in AAV9 capsid, with no other amino acid modifications to the capsid sequence. AAV.PHP.eB is a capsid having a TLAVPFK (SEQ ID NO:20) insertion in AAV9 capsid, with two amino acid modifications of the capsid sequence upstream of the PHP.B insertion (see also Table 10). Table 3A summarizes the capsids utilized in the study.

TABLE 3A

| | Parent | | Location of | | |
|---|---|---|---|---|---|
| Name | capsid | Mutation | insertion 2 | Peptide 2 | SEQ ID NO: |
| AAV9 | AAV9 | — | — | — | |
| PHP.B | AAV9 | — | 588_589 | TLAVPFK | 20 |
| PHP.eB | AAV9 | 586A_587Q | 588_589 | TLAVPFK | 20 |
| | | delinsDG | | | |
| AAV.hDyn | AAV9 | — | 588_589 | TLAAPFK | 1 |
| AAV.PHP.S | AAV9 | — | 588_589 | QAVRTSL | 16 |
| AAV.PHP.SH | AAV9 | — | 588_589 | QAVRTSH | 17 |

### Materials and Methods

Constructs of AAV9, AAV.PHPeB, AAV.hDyn, AAV.PHP.S and AAV.PHP.SH encoding GFP transgene were prepared and formulated in 1×PBS+0.001% Pluronic. Female C57BL/6 mice were randomized into treatment groups base on Day 1 bodyweight. Five groups of female C57BL/6 mice were each intravenously administered AAV9.GFP, AAV.PHPeB.GFP, AAV.hDyn.GFP, AAV.PHP.S.GFP or AAV.PHP.SH.GFP in accordance with Table 3B, below. The dosing volume was 10 mL/kg (0.200 mL/20 g mouse). The mice were 8-12 weeks of age at the start date. At day 15 post administration, the animals were euthanized, and peripheral tissues were collected, including brain tissue, liver, forelimb biceps, heart, kidney, lung, ovaries, and the sciatic nerve.

TABLE 3B

| Gr. | N | Agent | Formulation dose | Route | Schedule |
|---|---|---|---|---|---|
| 1 | 9 | AAV9 | 2.5E12 GC/kg | iv | day 1 |
| 2 | 5 | PHPeB | 2.5E12 GC/kg | iv | day 1 |
| 3 | 5 | hDyn | 2.5E12 GC/kg | iv | day 1 |
| 4 | 5 | PHP.S | 2.5E12 GC/kg | iv | day 1 |
| 5 | 5 | PHP.SH | 2.5E12 GC/kg | iv | day 1 |

Quantitiative PCR (qPCR) was used to determine the number of vector genomes per jig of brain genomic DNA. Brain samples from injected mice were processed and genomic DNA was isolated using Blood and Tissue Genomic DNA kit from Qiagen. The qPCR assay was run on a QuantStudio 5 instrument (Life Technologies Inc) using primer-probe combination specific for eGFP following a standard curve method.

The AAV vector genome copies per jig of brain genomic DNA was at least a log higher in mice that were administered AAV.hDyn compared to all other AAV serotypes: AAV9, AAV.PHPeB, PHP.S, and PHP.SH (see FIG. 8). As seen in this study, GC/μg genomic DNA is highest for AAV.hDyn, which is AAV9 capsid containing the “TLAAPFK” (SEQ ID NO:1) peptide insert (a peptide from human axonemal dynein) between residues 588-589 of the AAV9 capsid. The study demonstrated transduction in mouse brain at greater than 1E04 GC/jig transgene on average in 5 mice systemically administered AAV.hDyn carrying eGFP. Other modified AAV9 capsids, however, including the vector AAV.PHPeB, which contains the “TLAVPFK” (SEQ ID NO:20) sequence (a peptide from mouse dynein) demonstrated transduction in mouse brain at less than 1E03 GC/jig transgene upon systemic treatment.

### 6.10. Example 9—Construction of rAAV Capsid Containing LALGETTRPA (SEQ ID NO:9)

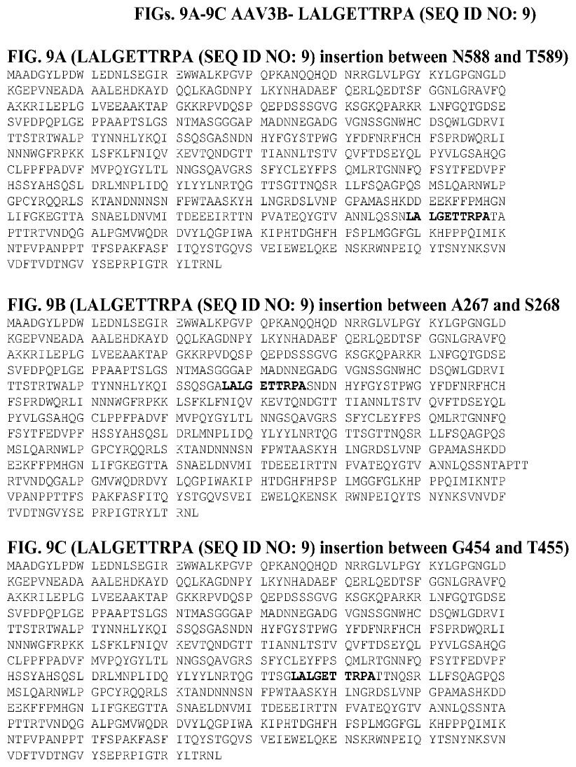

FIG. 9A depicts the amino acid sequence for a recombinant AAV3B vector capsid including a peptide insertion of amino acid sequence LALGETTRP (SEQ ID NO:9) between N588 and T589 of VR-IIIV. Inserted peptide in bold.

FIG. 9B depicts the amino acid sequence for a recombinant AAV3B vector capsid including a peptide insertion of amino acid sequence LALGETTRP (SEQ ID NO:9) between S267 and S268 of VR-III. Inserted peptide in bold.

FIG. 9C depicts the amino acid sequence for a recombinant AAV3B vector capsid including a peptide insertion of amino acid sequence LALGETTRP (SEQ ID NO:9) between G454 and T455 of VR-IV. Inserted peptide in bold.

### 6.11. Example 10—Construction of rAAV Capsid Containing LALGETTRPA (SEQ ID NO:9)

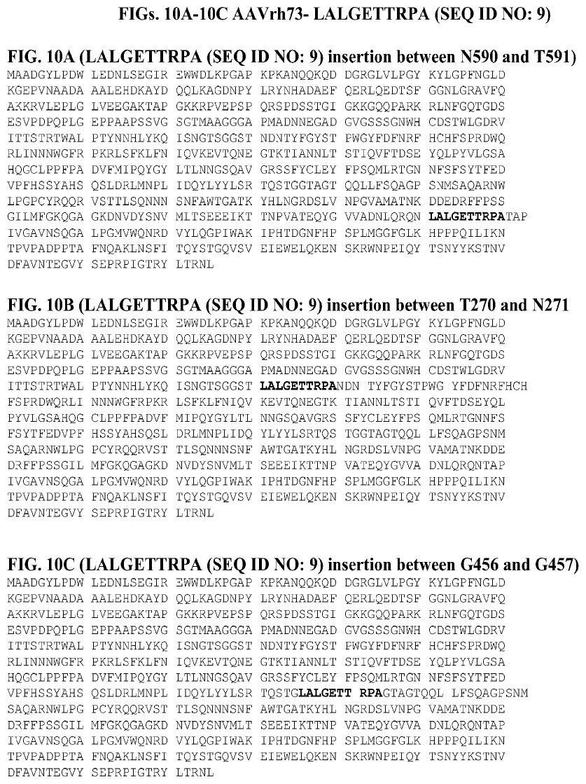

FIG. 10A depicts the amino acid sequence for a recombinant AAVrh73 vector capsid including a peptide insertion of amino acid sequence LALGETTRP (SEQ ID NO:9) between N590 and T591 of VR-IIIV. Inserted peptide in bold.

FIG. 10B depicts the amino acid sequence for a recombinant AAVrh73 vector capsid including a peptide insertion of amino acid sequence between T270 and N271 of VR-III. Inserted peptide in bold.

FIG. 10C depicts the amino acid sequence for a recombinant AAVrh73 vector capsid including a peptide insertion of amino acid sequence LALGETTRPA (SEQ ID NO:9) between G456 and G457 of VR-IV. Inserted peptide in bold.

### 6.12. Example 11—Construction of rAAV Capsid Containing LALGETTRPA (SEQ ID NO:9)

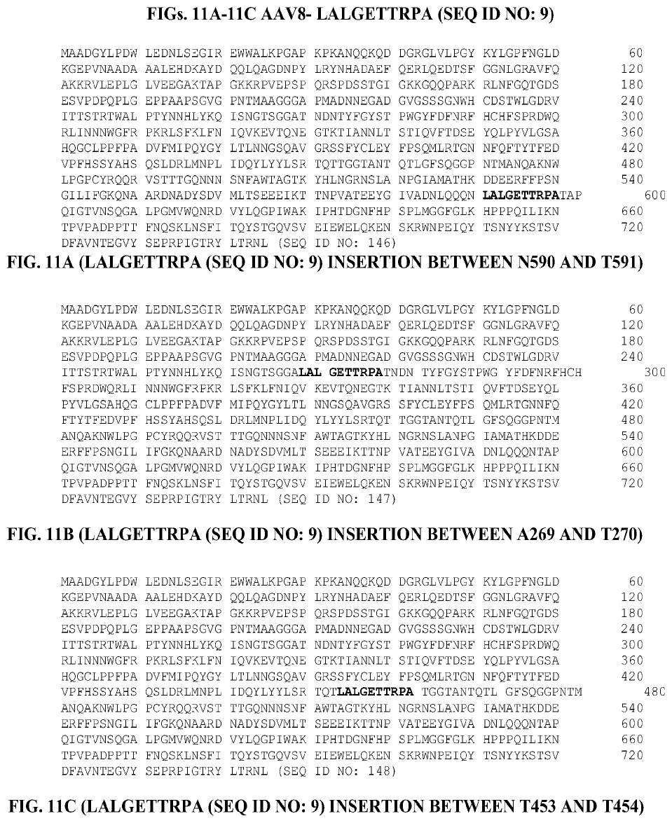

FIG. 11A depicts the amino acid sequence for a recombinant AAV8 vector capsid including a peptide insertion of amino acid sequence LALGETTRP (SEQ ID NO:9) between N590 and T591 of VR-VIII. Inserted peptide in bold.

FIG. 11B depicts the amino acid sequence for a recombinant AAV8 vector capsid including a peptide insertion of amino acid sequence LALGETTRP (SEQ ID NO:9) between A269 and T270 of VR-III. Inserted peptide in bold.

FIG. 11C depicts the amino acid sequence for a recombinant AAV8 vector capsid including a peptide insertion of amino acid sequence LALGETTRP (SEQ ID NO:9) between T453 and T454 of VR-IV. Inserted peptide in bold.

### 6.13. Example 12—In Vitro Testing of Transduction an Crossing Blood Brain Barrier

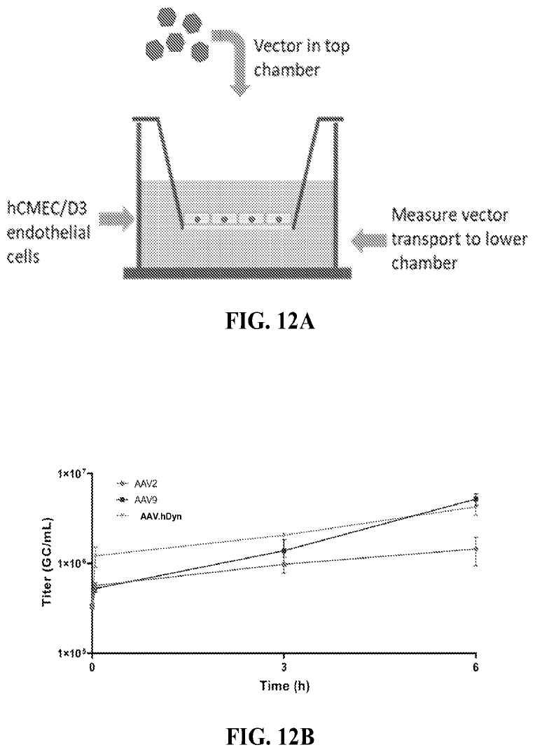

The ability of the modified capsids to cross the blood brain barrier was tested in an in vitro transwell assay using hCMEC/D3 BBB cells (SCC066, Millipore-Sigma) (see FIGS. 12A-12B). More specifically, the assay was essentially adapted from Sade, H. et al. (2014 PLoS ONE 9(4): e96340) A human Blood-Brain Barrier transcytosis assay reveals Antibody Transcytosis influenced by pH-dependent Receptor Binding, April 2014, Vol. 9, Issue 4; and Zhang, X., Blood-brain barrier shuttle peptides enhance AAV transduction in the brain after systemic administration, 2018 Biomaterials 176: 71-83. Briefly, 5×10⁴ hCMEC/D3 cells/cm 2 were seeded in collagen-coated transwell inserts in a 12-well plate. Each insert contained 500 μL media and the lower chamber contained 1 mL media. Media was replaced every second day. The supernatant was removed at 10 days post-seeding (the zero (0) timepoint). At this 0 timepoint, the cells were transduced by adding 1×10⁹ GC of vector to the upper insert chamber media. 10 μL lower chamber supernatant samples were removed for testing at intervals 0.5, 3, 6, and 23 hours post-transduction. Each condition (vector) was tested in duplicate, and measured for titer via qPCR against PolyA in triplicate.

FIGS. 12A-12B depict an in vitro transwell assay for AAV.hDyn (AAV9 with TLAAPFK (SEQ ID NO:1) between amino acid residues 588-589) crossing a blood brain barrier (BBB) cell layer (FIG. 12A), and results showing that AAV.hDyn (indicated by inverted triangles in the figure) crosses the BBB cell layer of the assay faster than AAV9 (squares), as well as faster and to a greater extent than AAV2 (circles) (FIG. 12B). The developed in vitro assay predicted enhanced BBB cross-trafficking and similar assays can be used to predict targeting to other organs as well.

### 6.14. Example 13—Transduction and Biodistribution of Modified Capsids

6.14.1 Materials and Methods

Capsid modifications were performed on widely used AAV capsids including AAV8, AAV9, and AAVrh.10 by inserting various peptide sequences after the position S454 of the VR-IV (Table 4) or after position Q588 of the VR-VIII surface exposed loop of the AAV capsid, as well as insertions after the initiation codon of VP2, which begins at amino acid 137 (AAV4, AAV4-4, and AAV5) or at amino acid 138 (AAV1, AAV2, AAV3, AAV3-3, AAV6, AAV7, AAV8, AAV9, AAV9e, rh.10, rh.20, rh.39, rh.74, and hu.37) (FIG. 7) (see also Table for certain capsid sequences). Selected single to multiple amino acid mutations were also used for modifying the capsids. See also, Yost et al., Structure-guided engineering of surface exposed loops on AAV Capsids. 2019. ASGCT Annual Meeting; and Wu et al., 2000 J. Virology (supra). It was confirmed that packaging efficiency was not negatively impacted following any of these capsid modifications in small scale.

rAAVs with certain modified capsids were tested for transduction in vitro in Lec2 cells as described above in Example 5. Modified AAVs tested for transduction in Lec2 cells as follows: eB 588 Ad, eB 588 Hep, eB 588 p79, eB 588 Rab, AAV9 588 Ad, AAV9 588 Hep, AAV9 588 p79, AAV9 588 Rab, eB VP2 Ad, eB VP2 Hep, eB VP2 p79, eB VP2 Rab, AAV9 VP2 Ad, AAV9 VP2 Hep, AAV9 VP2 p79, AAV9 VP2 Rab as compared to AAV9. See Table 4B below for identity of AAV capsids.

To test biodistribution, modified AAVs were packaged with an eGFP transgene cassette containing specific barcodes corresponding to each individual capsid. Novel barcoded vectors were pooled and injected into mice in order to increase the efficiency of screening.

To analyse the bio-distribution of genetically altered AAV vectors, various vectors encoding GFP were prepared and formulated in 1×PBS+0.0001% Pluronic acid. All vectors were made with cis plasmids containing a ten (10) bp barcode to enable next-generation sequencing (NGS) library (pool) preparation. Three (3) vector pools (Study 1, Study 2 and Study 3 vectors) were injected intravenously into a cohort of 5 female C57Bl/6 mice in accordance with Tables 4A-C. The dosing volume was 10 mL/kg (0.2 mL/20 g mouse) for each.

The mice were randomized into treatment groups based on Day 1 bodyweight and their age at start date was 8-12 weeks. At day 15 post administration, the animals were euthanized and peripheral tissues were collected, including brain, kidney, liver, sciatic nerve, lung, heart, and muscle tissue. In the studies where selected capsids from the pool were injected individually, the same protocol was followed

Genomic DNA (gDNA) was isolated from tissue samples using DNeasy Blood and Tissue kit (69506) from Qiagen. Each vector's barcode region was amplified with primers containing overlaps for NGS and unique dual indexing (UDI) and multiplex sequencing strategies, as recommended by the manufacturer (Illumina). Illumina MiSeq using reagent nano and micro kits v2 (MS-103-1001/1002) were used to determine the relative abundance of each barcoded AAV vector per sample collected from the mice. Accordingly, each vector sample in Tables 4A-C below was barcoded as noted above to allow for each read to be identified and sorted before the final data analysis. The data was normalized based on the composition of AAVs in the originally injected pool and quantified using the total genome copy number obtained from qPCR analysis with a primer-probe combination specific to the barcoded sample.

TABLE 4A

| | | | Insertion | | |
|---|---|---|---|---|---|
| Study 1 | Name | Capsid | Point | Peptide | Notes |
| BC01 | AAV9 | AAV9 | — | — | |
| BC02 | PHP.eB | PHP.eB | 588_589 | TLAVPFK | |
| | | | | (SEQ ID | |
| | | | | NO: 20) | |
| BC03 | AAV8.BBB | Modified | — | — | A269S |
| | | AAV8 | | | |
| BC04 | AAV9.BBB | Modified | — | — | S263G/S269T/ |
| | | AAV9 | | | A273T |
| BC05 | AAV8.BBB.LD | Modified | — | — | A269S, 498- |
| | | AAV8 | | | NNN/AAA-500 |
| BC06 | AAV9.BBB.LD | Modified | — | — | S263G/S269T/ |
| | | AAV9 | | | A273T, 496- |
| | | | | | NNN/AAA-498 |
| BC07 | rh.10 | rh.10 | — | — | |
| BC08 | rh.10.LD | Modified | — | — | 498-NNN/AAA- |
| | | rh.10 | | | 500 |
| BC09 | AAV.hDyn | modifiedAAV9 | 588_589 | TLAAPFK | |
| | | | | (SEQ ID | |
| | | | | NO: 1) | |
| BC10 | PHP.S | PHP.S | 588_589 | QAVRTSL | — |
| | | | | (SEQ ID | |
| | | | | NO: 163) | |
| BC11 | PHP.SH | PHP.SH | 588_589 | QAVRTSH | — |
| | | | | (SEQ ID | |
| | | | | NO: 17) | |
| BC13 | rh39 | rh.39 | — | — | — |

TABLE 4B

| | | | Insertion | | |
|---|---|---|---|---|---|
| Study 2 | Name | Capsid | Point | Peptide | Notes |
| BC20 | eB 588 Ad | PHP.eB | 588_589 | SITLVKSTQTV | Replaces |
| | | | | (SEQ ID NO: 14) | TLAVPFK peptide |
| | | | | | (SEQ ID NO: 20) |
| BC21 | eB 588 Hep | PHP.eB | 588_589 | TILSRSTQTG (SEQ | Replaces |
| | | | | ID NO: 15) | TLAVPFK peptide |
| | | | | | (SEQ ID NO: 20) |
| BC22 | eB 588 p79 | PHP.eB | 588_589 | VVMVGEKPITITQ | Replaces |
| | | | | HSVETEG (SEQ ID | TLAVPFK peptide |
| | | | | NO: 18) | (SEQ ID NO: 20) |
| BC23 | eB 588 Rab | PHP.eB | 588_589 | RSSEEDKSTQTT | Replaces |
| | | | | (SEQ ID NO: 19) | TLAVPFK peptide |
| | | | | | (SEQ ID NO: 20) |
| BC24 | 9 588 Ad | AAV9 | 588_589 | SITLVKSTQTV | |
| | | | | (SEQ ID NO: 14) | |
| BC25 | 9 588 ?ep | AAV9 | 588_589 | TILSRSTQTG (SEQ | |
| | | | | ID NO: 15) | |
| BC26 | 9 588 p79 | AAV9 | 588_589 | VVMVGEKPITITQ | |
| | | | | HSVETEG (SEQ ID | |
| | | | | NO: 18) | |
| BC27 | 9 588 Rab | AAV9 | 588_589 | RSSEEDKSTQTT | |
| | | | | (SEQ ID NO: 19) | |
| BC28 | eB VP2 Ad | PHP.eB | 138_139 | SITLVKSTQTV | Also has |
| | | | | (SEQ ID NO: 14) | TLAVPFK (SEQ |
| | | | | | ID NO: 20) insert |
| | | | | | after residue 588 |
| BC29 | eB VP2 Hep | PHP.eB | 138_139 | TILSRSTQTG (SEQ | Also has |
| | | | | ID NO: 15) | TLAVPFK (SEQ |
| | | | | | ID NO: 20) insert |
| | | | | | after residue 588 |
| BC30 | eB VP2 p79 | PHP.eB | 138_139 | VVMVGEKPITITQ | Also has |
| | | | | HSVETEG (SEQ ID | TLAVPFK (SEQ |
| | | | | NO: 18) | ID NO: 20) insert |
| | | | | | after residue 588 |
| BC31 | AAV9 | AAV9 | — | — | |
| BC32 | eB VP2 Rab | PHP.eB | 138_139 | RSSEEDKSTQTT | Also has |
| | | | | (SEQ ID NO: 19) | TLAVPFK (SEQ |
| | | | | | ID NO: 20) insert |
| | | | | | after residue 588 |
| BC33 | 9 VP2 Ad | AAV9 | 138_139 | SITLVKSTQTV | |
| | | | | (SEQ ID NO: 14) | |
| BC34 | 9 VP2 Hep | AAV9 | 138_139 | TILSRSTQTG (SEQ | |
| | | | | ID NO: 15) | |
| BC35 | 9 VP2 p79 | AAV9 | 138_139 | VVMVGEKPITITQ | |
| | | | | HSVETEG (SEQ ID | |
| | | | | NO: 18) | |
| BC36 | 9 VP2 Rab | AAV9 | 138_139 | RSSEEDKSTQTT | |
| | | | | (SEQ ID NO: 19) | |

TABLE 4C

| | | | Insertion | | |
|---|---|---|---|---|---|
| Study 3 | Name | Capsid | Point | Peptide | Notes |
| BC01 | AAV9 | AAV9 | — | — | |
| BC03 | AAV8-BBB | AAV8 | — | — | A269S |
| BC07 | rh10 | rh.10 | — | — | |
| BC09 | AAV.hDyn | AAV.hDyn | 588_589 | TLAAPFK | |
| | | | | (SEQ ID NO: 1) | |
| BC12 | PHP.B | PHP.B | 588_589 | TLAVPFK | |
| | | | | (SEQ ID | |
| | | | | NO: 20) | |
| BC20 | AAV9 S454- | AAV9 | 454_455 | DDDDDDDD | |
| | D8 | | | (SEQ ID NO: 2) | |
| BC22 | AAV9 S454- | AAV9 | 454_455 | LSSRLDA | |
| | Brain 1 | | | (SEQ ID NO: 3) | |
| BC23 | AAV9 S454- | AAV9 | 454_455 | CLSSRLDAC | |
| | Brian 1C | | | (SEQ ID NO: 4) | |
| BC24 | AAV9 S454- | AAV9 | 454_455 | LPVAS (SEQ | |
| | Kidney 1 | | | ID NO: 6) | |
| BC25 | AAV9 S454- | AAV9 | 454_455 | CLPVASC | |
| | Kidney 1C | | | (SEQ ID NO: 5) | |
| BC26 | AAV9 S454- | AAV9 | 454_455 | ASSLNIA | |
| | Muscle1 | | | (SEQ ID NO: 7) | |
| BC27 | AAV9 S454- | AAV9 | 454_455 | HAIYPRH | |
| | TfR1 | | | (SEQ ID | |
| | | | | NO: 10) | |
| BC29 | AAV9 S454- | AAV9 | 454_455 | RTIGPSV | |
| | TfR3 | | | (SEQ ID | |
| | | | | NO: 12) | |
| BC30 | AAV9 S454- | AAV9 | 454_455 | CRTIGPSVC | |
| | TfR4 | | | (SEQ ID | |
| | | | | NO: 13) | |
| BC31 | AAV9 S454- | AAV9 | 454_455 | DYKDDDDK | |
| | FLAG | | | (SEQ ID | |
| | | | | NO: 21) | |
| BC37 | pRGX1005- | PHP.eB | 588_589 | TLAVPFK | |
| | PHP.eB (no | | | (SEQ ID | |
| | BC) | | | NO: 20) | |

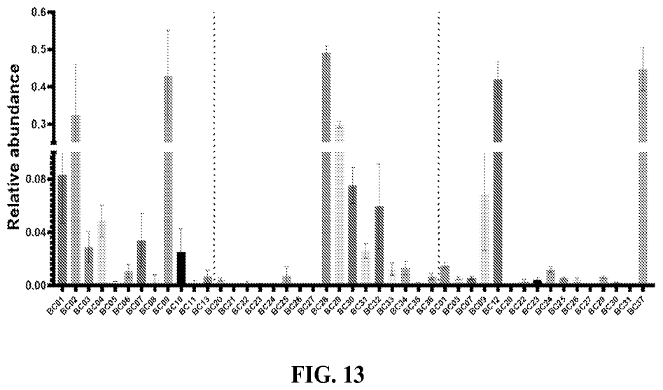

In the studies where selected capsids from the pool were injected individually, qPCR was used to determine the number of vector genomes per lag of tissue genomic DNA. qPCR was done on a QuantStudio 5 (Life Technologies, Inc.) using primer-probe combination specific for eGFP following a standard curve method (FIG. 13).

From the study where individual vectors were injected into mice for characterization, formalin fixed mouse brains were sectioned at 40 μm thickness on a vibrating blade microtome (VT1000S, Leica) and the floating sections were probed with antibodies against GFP to look at the cellular distribution of the delivered vectors.

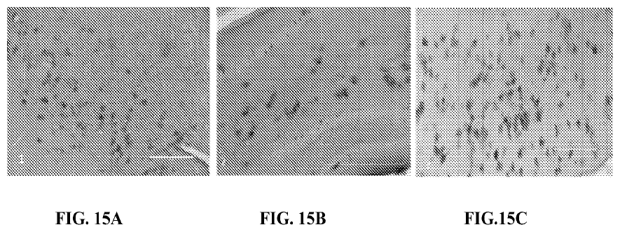

More specifically, fixed brains from the mice injected with AAV.hDyn were sectioned using a Vibratome (Leica, VT-1000) and the GFP expression was evaluated using an anti-GFP antibody (AB3080, Millipore Sigma), Vectastain ABC kit (PK-6100, Vector Labs) and DAB Peroxidase kit (SK-4100, Vector Labs). Broad distribution of GFP expressing cells were present throughout the brain in mice injected with AAV.hDyn, including distribution in the cortex, striatum, and hippocampus of the brain. FIGS. 15A-15C show the images from these regions and the scale bar is 400 um (discussed below).

6.14.2 Results

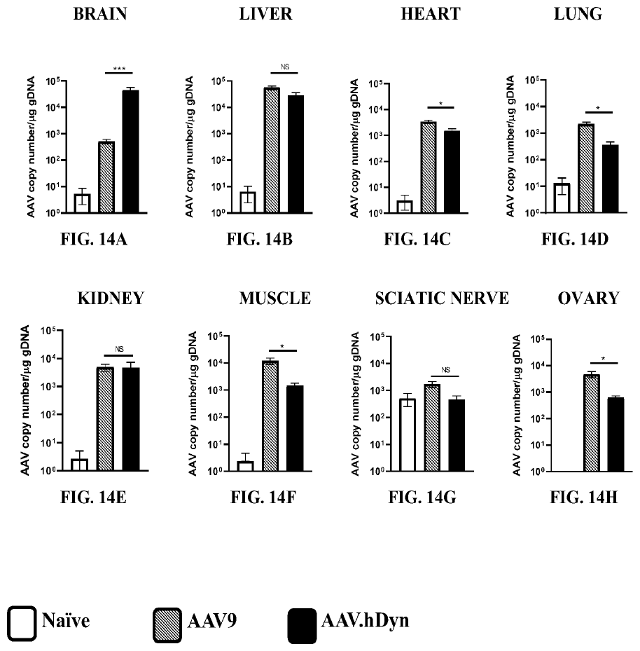

Results are shown in FIG. 13, FIGS. 14A-14H, and FIGS. 15A-15C.

Data for the Lec2 cell transduction assay not shown. The AAV9 588 Hep (AAV9 with the peptide TILSRSTQTG (SEQ ID NO:15) inserted after position 588) exhibited significantly greater transduction (4-fold) than wild type AAV9, and AAV9 VP2 Ad (AAV9 with the peptide SITLVKSTQTV (SEQ ID NO:14) inserted after position 138), AAV9 VP2 Hep (AAV9 with the peptide TILSRSTQTG (SEQ ID NO:15) inserted after position 138), and AAV9 VP2 Rab (AAV9 with the peptide RSSEEDKSTQTT (SEQ ID NO:19) inserted after position 138) exhibited slightly greater transduction of the Lec2 cells relative to AAV9. The other AAVs assayed exhibited lower levels of transduction than AAV9.

FIG. 13 depicts results of Next Generation Sequencing (NGS) analysis of brain gDNA, revealing relative abundances (percent composition) of the capsid pool delivered to mouse brains following intravenous injection. The data was normalized based on the composition of AAVs in the originally injected pool and quantified using the total genome copy number obtained from qPCR analysis with a primer-probe combination specific to the eGFP sequence. Data shown are from three different experiments. Dotted lines indicate which vectors were pooled together. Parental AAV9 was used as standard and included in each pool. The “BC” identifiers are as indicated in Tables 4A, 4B and 4C above.

FIGS. 14A-14H depict an in vivo transduction profile of AAV.hDyn in female C57Bl/6 mice, showing copy number/microgram gDNA in naïve mice, or mice injected with either AAV9 or AAV.hDyn in brain (FIG. 14A), liver (FIG. 14B), heart (FIG. 14C), lung (FIG. 14D), kidney (FIG. 14E), skeletal muscle (FIG. 14F), sciatic nerve (FIG. 14G), and ovary (FIG. 14H), where AAV.hDyn shows increased brain bio-distribution compared to AAV9. The AAV vector genome copies per mg of brain genomic DNA was at least a log higher in mice that were administered AAV.hDyn compared to the parental AAV9 vector.

FIGS. 15A-15C show images from the regions analysed in the Immunohistochemical Analysis described above; scale bar is 400 FIGS. 15A-15C depict distribution of GFP from AAV.hDyn throughout the brain, where images of immunohistochemical staining of brain sections from the striatum (FIG. 15A), hippocampus (FIG. 15B), and cortex (FIG. 15C) revealed a global transduction of the brain by the modified vector.

6.14.3 Conclusions

AAV capsid modifications performed either by insertions in surface exposed loops of VR-IV and VR-VIII or by specific amino acid mutations did not affect their packaging efficiency and were able to produce similar titers in the production system described herein.

Intravenous administration of AAV.hDyn to mice resulted in higher relative abundance of the viral genome and greater brain cell transduction than other modified AAV vectors and AAV9 tested.

### 6.15. Example 14—Biodistribution of an rAAV Vector Pool in Cynomolgus Monkeys after IVT Injection

The administration, in vivo and post-mortem observations, and biodistribution of a pool of recombinant AAVs having engineered capsids and a GFP transgene was evaluated following a single intravitreal injection (IVT) in cynomolgus monkeys (Table 5). The pool contained multiple capsids each of which contained a unique barcode identification allowing identification using next generation sequencing (NGS) analysis following administration to cynomolgus monkeys. All animals on this study were naïve with respect to prior treatment. The pool may comprise at least the following recombinant AAVs having the engineered capsids listed in Table 5.

TABLE 5
Recombinant AAVs for Cynomolgus Monkey Study

| | | | | | Peptide |
|---|---|---|---|---|---|
| | | Capsid | Location of | | SEQ ID |
| Name | Capsid | modification | insertion | Peptide | NO: |
| AAV8 | AAV8 | — | — | — | |
| AAV8.BBB | Modified | A269S | — | — | |
| | AAV8 | | | | |
| AAV8.BBB.LD | Modified | A269S, 498- | — | — | |
| | AAV8 | NNN/AAA-500 | | | |
| AAV9 | AAV9 | — | — | — | |
| AAV9 S454- | AAV9 | — | 454_455 | LSSRLDA | 3 |
| Brain1 | | | | | |
| AAV9 S454- | AAV9 | — | 454_455 | CLSSRLDAC | 4 |
| Brain 1C | | | | | |
| AAV9 S454-D8 | AAV9 | — | 454_455 | DDDDDDDD | 2 |
| AAV9 S454- | AAV9 | — | 454_455 | LPVAS | 6 |
| Kidney 1 | | | | | |
| AAV9 S454- | AAV9 | — | 454_455 | CLPVASC | 5 |
| Kidney 1C | | | | | |
| AAV9 S454- | AAV9 | — | 454_455 | ASSLNIA | 7 |
| Muscle1 | | | | | |
| AAV9 S454- | AAV9 | — | 454_455 | HAIYPRH | 10 |
| Tfr1 | | | | | |
| AAV9 S454- | AAV9 | — | 454_455 | RTIGPSV | 12 |
| Tfr3 | | | | | |
| AAV9 S454- | AAV9 | — | 454_455 | CRTIGPSVC | 13 |
| TfR3C | | | | | |
| AAV9.496NNN/ | Modified | 498-NNN/AAA- | — | — | |
| AAA498 | AAV9 | 500 | | | |
| AAV9.496NNN/ | Modified | 498-NNN/AAA- | — | — | |
| AAA498.W503R | AAV9 | 500, W503R | | | |
| AAV9.588Ad | AAV9 | — | 588_589 | SITLVKSTQ | 14 |
| | | | | TV | |
| AAV9.588Herp | AAV9 | — | 588_589 | TILSRSTQT | 15 |
| | | | | G | |
| AAV9.BBB | Modified | S263G/S269T/ | — | — | |
| | AAV9 | A273T | | | |
| AAV9.BBB.LD | Modified | S263G/S269T/ | — | — | |
| | AAV9 | A273T, 496- | | | |
| | | NNN/AAA-498 | | | |
| AAV9.Q474A | Modified | Q474A | — | — | |
| | AAV9 | | | | |
| AAV9.W503R | Modified | W503R | — | — | |
| | AAV9 | | | | |
| AAVPHPeB. | PHP.eB | — | 138_139 | SITLVKSTQ | 14 |
| VP2Ad | | | | TV | |
| AAVPHPeB. | PHP.eB | — | 138_139 | TILSRSTQT | 15 |
| VP2Herp | | | | G | |
| PHP.B | AAV9 | — | 588_589 | TLAVPFK | 20 |
| PHP.eB | Modified | A587D, Q588G | 588_589 | TLAVPFK | 20 |
| | PHP.B | | | | |
| PHP.hB | — | — | — | — | — |
| PHP.S | AAV9 | — | 588_589 | QAVRTSL | 16 |
| PHP.SH | AAV9 | — | 588_589 | QAVRTSH | 17 |
| AAV1 | AAV1 | — | — | — | — |
| AAV2 | AAV2 | — | — | — | — |
| AAV2.7m8 | AAV2.7m8 | — | — | — | — |
| AAV3B | AAV3B | — | — | — | — |
| AAV4 | AAV4 | — | — | — | — |
| AAV5 | AAV5 | — | — | — | — |
| AAV6 | AAV6 | — | — | — | — |
| AAV7 | AAV7 | — | — | — | — |
| hu.12 | hu.12 | — | — | — | — |
| hu.13 | hu.13 | — | — | — | — |
| hu.21 | hu.21 | — | — | — | — |
| hu.26 | hu.26 | — | — | — | — |
| hu.51 | hu.51 | — | — | — | — |
| hu.53 | hu.53 | — | — | — | — |
| hu.56 | hu.56 | — | — | — | — |
| rh.31 | rh.31 | — | — | — | — |
| hu.31 | hu.31 | — | — | — | — |
| rh.34 | rh.34 | — | — | — | — |

6.15.1 Study Design

Three female cynomolgus animals were used. Relevant tissues were collected to evaluate the biodistribution (measured by NGS and PCR) associated with IVT injection. Three animals received a single intravitreal injection.

The intravitreal (IVT) injection was administered bilateral as a bolus injection at a dose volume of 50 μL.

6.15.2 Observations and Examinations

Clinical signs were recorded at least once daily beginning approximately two weeks prior to initiation of dosing and continuing throughout the study period. The animals were observed for signs of clinical effects, illness, and/or death.

Ophthalmological examinations were performed on animals prior to dose administration, and on Days 2, 8, 15 and 22. All animals were sedated with ketamine hydrochloride IM for the ophthalmologic examinations performed following Day 1. For the examinations on Day 1, the animals were sedated with injectable anesthesia (refer to Section 15.3.3). The eyes were dilated with 1% tropicamide prior to the examination. The examination included slit-lamp biomicroscopy and indirect ophthalmoscopy. Additionally, applanation tonometry was performed on animals prior to dosing, immediately following dose administration (˜10 to 15 minutes) and on Days 2 and 22.

Blood samples (˜3 mL) were collected from a peripheral vein for neutralizing antibodies analysis approximately 2 to 3 weeks prior to dose administration.

6.15.3 Bioanalytical Sample Collection

Blood samples (˜5 mL) were collected from fasted animals from a peripheral vein for PBMC analysis prior to dose administration (Day 1), on Days 8 and 15 and prior to necropsy (Day 22). The samples were obtained using lithium heparin tubes and the times recorded.

Blood samples were collected from a peripheral vein for bioanalytical analysis prior to dose administration (Day 1, 2 mL) and necropsy (Day 22, 5 mL). The samples were collected in clot tubes and the times recorded. The tubes were maintained at room temperature until fully clotted, then centrifuged at approximately 2400 rpm at room temperature for 15 minutes. The serum was harvested, placed in labeled vials (necropsy sample split into 1 mL aliquots), frozen in liquid nitrogen, and stored at −60° C. or below.

6.15.4 Necroscopy

A gross necropsy was performed on any animal found dead or sacrificed moribund, and at the scheduled necropsy, following at least 21 days of treatment (Day 22). All animals, except those found dead, were sedated with 8 mg/kg of ketamine HCl IM, maintained on an isoflurane/oxygen mixture and provided with an intravenous bolus of heparin sodium, 200 IU/kg. The animals were perfused via the left cardiac ventricle with 0.001% sodium nitrite in saline.

The following tissues were saved from all animals: Bone marrow, brain, cecum, colon, dorsal nerve roots and ganglion, duodenum, esophagus, eyes with optic nerves, gross lesions, heart, ileum, jejunum, kidneys, knee joint, liver, lungs with bronchi, lymph nodes, ovaries, pancreas, sciatic nerve, skeletal muscle, spinal cord, spleen, thyroids, trachea, and vagus nerve.

6.15.5 Bioanalytical Analysis

The vector copy number and number of transcripts in tissues was examined by quantitative PCR and NGS methods.

6.15.6 Results

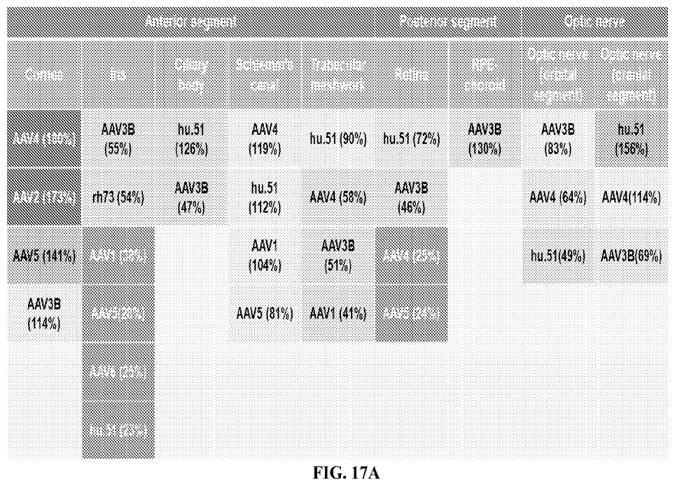

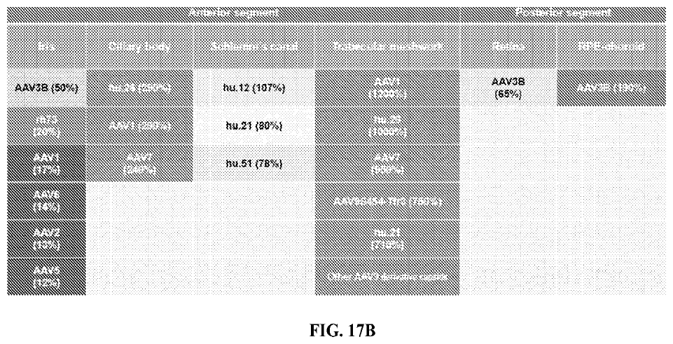

FIG. 17A depicts results of Next Generation Sequencing (NGS) analysis of different structures and cellular components of the eye (see FIGS. 16A and 16B for eye anatomy), revealing relative abundances (percent composition) of the capsid pool following IVT. The data was normalized based on the composition of AAVs in the originally injected pool and quantified using the total genome copy number obtained from qPCR analysis with a primer-probe combination specific to the eGFP sequence. AAV2.7m8 was used as standard and data shows top performing capsids relative to the AAV2.7m8 capsid.

FIG. 17B depicts number of transcripts (RNA) in different tissues of the eye, revealing relative abundances (percent composition) of the capsid pool following IVT. The data was normalized based on the composition of AAVs in the originally injected pool and quantified using the total genome copy number obtained from qPCR analysis with a primer-probe combination specific to the eGFP sequence. AAV2.7m8 was used as standard and data shows top performing capsids relative to the AAV2.7m8 capsid.

### 6.16. Example 15—Biodistribution of Modified Capsids in Cynomolgus Monkeys after IVT Injection

The administration, in vivo and post-mortem observations, and biodistribution of the top hit recombinant AAVs from the barcoded library screen in NHPs will be evaluated following a single intravitreal injection (IVT) in cynomolgus monkeys (Table 6). All animals on this study were naïve with respect to prior treatment.

TABLE 6
Recombinant AAVs for IVT Cynomolgus Monkey Study

| | | Location of | | Peptide |
|---|---|---|---|---|
| Name | Capsid | insertion | Peptide | SEQ ID NO: |
| AAV2.7m8.455 | AAV2 | T455_T456 | LALGETTRPA | 9 |
| AAV2.7m8.588 | AAV2 | N587_R588 | LALGETTRPA | 9 |
| AAV3B | AAV3B | — | — | — |
| AAV3B.7m8 | AAV3B | G454_T455 | lALGETTRPA | 9 |
| AAVrh73 | AAVrh73 | — | — | — |
| AAVrh73.7m8 | AAVrh73 | G456_T457 | LALGETTRPA | 9 |

6.16.1 Study Design

Two female cynomolgus animals will be used per capsid. Relevant tissues will be collected to evaluate the biodistribution associated with the different AAVs using IVT injection (see FIGS. 16A and B). The IVT injection will be administered bilateral as a bolus injection at a dose volume of 50 μL.

6.16.2 Observations and Examinations

Clinical signs will be recorded at least once daily beginning approximately two weeks prior to initiation of dosing and continuing throughout the study period. The animals will be observed for signs of clinical effects, illness, and/or death.

Ophthalmological examinations will be performed on animals prior to dose administration, and on Days 2, 8, 15 and 22. All animals will be sedated with ketamine hydrochloride IM for the ophthalmologic examinations performed following Day 1. For the examinations on Day 1, the animals will be sedated with injectable anesthesia.

The eyes will be dilated with 1% tropicamide prior to the examination. The examination will include slit-lamp biomicroscopy, indirect ophthalmoscopy, fundus imaging, and OCT at selected time points.

Blood samples (˜3 mL) will be collected from a peripheral vein for neutralizing antibodies analysis approximately 2 to 3 weeks prior to dose administration.

6.16.3 Bioanalytical Sample Collection

Blood samples will be collected from a peripheral vein for bioanalytical analysis prior to dose administration (Day 1, 2 mL) and necropsy (Day 28, 5 mL). The samples will be collected in clot tubes and the times recorded. The tubes will be maintained at room temperature until fully clotted, then centrifuged at approximately 2400 rpm at room temperature for 15 minutes. The serum will be harvested, placed in labeled vials (necropsy sample split into 1 mL aliquots), frozen in liquid nitrogen, and stored at −60° C. or below.

6.16.4 Necroscopy

A gross necropsy will be performed on any animal found dead or sacrificed moribund, and at the scheduled necropsy, following at least 21 days of treatment (Day 22). All animals, except those found dead, will be sedated with 8 mg/kg of ketamine HCl IM, maintained on an isoflurane/oxygen mixture and provided with an intravenous bolus of heparin sodium, 200 IU/kg. The animals will be perfused via the left cardiac ventricle with 0.001% sodium nitrite in saline.

Eyes will be collected at study end. One eye will be used for immunohistochemistry (IHC) and the other eye for biodistribution studies. Peripheral tissues may be collected.

6.16.5 Bioanalytical Analysis

The vector copy number and number of transcripts in the eye will be examined by quantitative PCR. GFP expression levels and localization will be examined using IHC.

### 6.17. Example 16—Biodistribution of an rAAV Vector Pool in Cynomolgus Monkeys after IVT Injection

Pooled barcoded vectors were administered to NHPs by intravitreal injection and biodistribution of vector DNA and RNA was assessed at sacrifice 3 weeks after the administration using the protocol described in Examples 14 and 15, infra. In particular, the vector pools were administered to 2 groups of 2 adult cynomolgus monkeys (Macaca fascicularis) in both eyes (bilaterally) by IVT according to Table 7 below:

TABLE 7

| | | Test | Dose | Dose | Endotoxin | | |
|---|---|---|---|---|---|---|---|
| Groups | NAB Titers | Article | (GC/eye) | Volume | (EU/ml) | Inflammation | Treatment |
| 1 | AAV2&9 < 1:2 | AAV- | 3 × 10¹⁰ | 50 μL | 0.058 | No | No |
| | AAV8 = 1:2 | NAV- | | per eye | | | |
| | AAV2 = 1:6 | GFPbc- | | 50 μL | | OS: mild; | No |
| | AAV8&9 < 1:2 | IVT1 | | per eye | | OD: | |
| | | | | | | moderate | |
| 2 | AAV2 = 1:2 | AAV- | 4 × 10¹¹ | 50 μL | <0.05 | OD: mild | No |
| | AAV8&9 < 1:2 | NAV- | | per eye | | | |
| | AAV2 = 1:2 | GFPbc- | | 50 μL | | OU: | Single |
| | AAV8&9 < 1:2 | IVT2 | | per eye | | moderate | dose |
| | | | | | | | DepoMedrol |
| | | | | | | | (IM); |
| | | | | | | | daily |
| | | | | | | | topical |
| | | | | | | | steroid |
| | | | | | | | drops |

Ophthalmological examinations were performed prior to dose administration and on days 2, 8, 15 and 22, which included slit-lamp biomicroscopy, indirect ophthalmoscopy and IOP measurement. At 3 week sacrifice, tissues were dissected (see FIGS. 16A and B for eye anatomy) and samples were collected in tubes containing RNAlater and stored refrigerated. The abundance of vector DNA and transcribed transgene RNA were assessed in the tissue samples relative to reference capsids AAV8 and AAV9 using ordinary one-way ANOVA with post hoc Dunnett's multiple comparisons generated using prism.

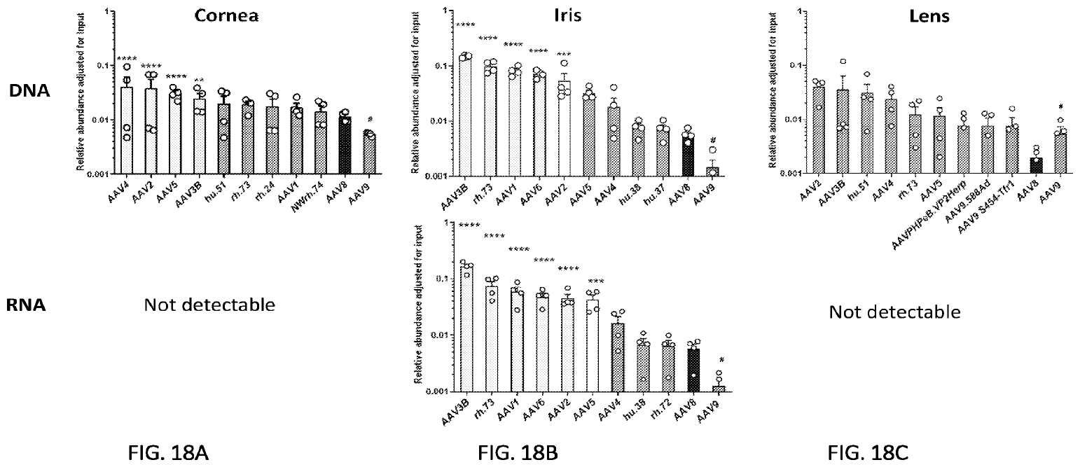

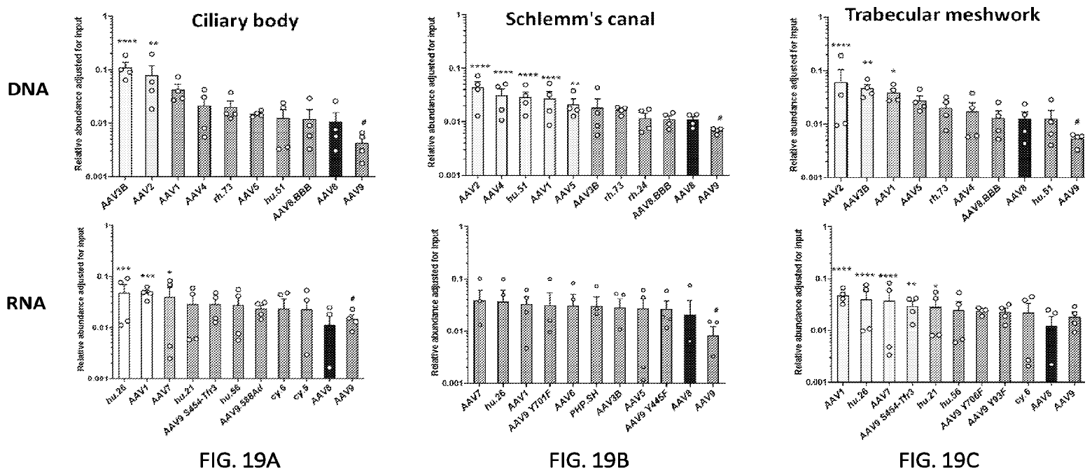

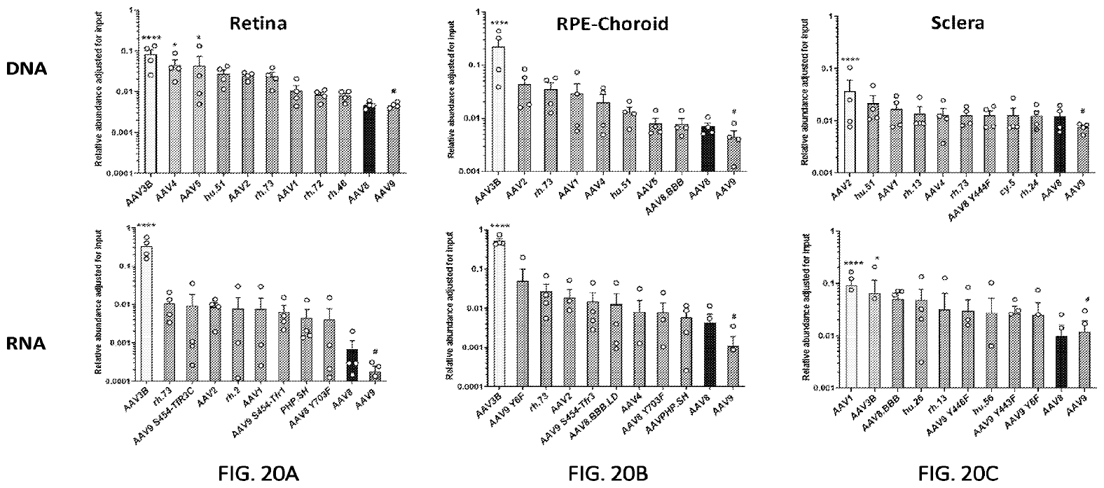

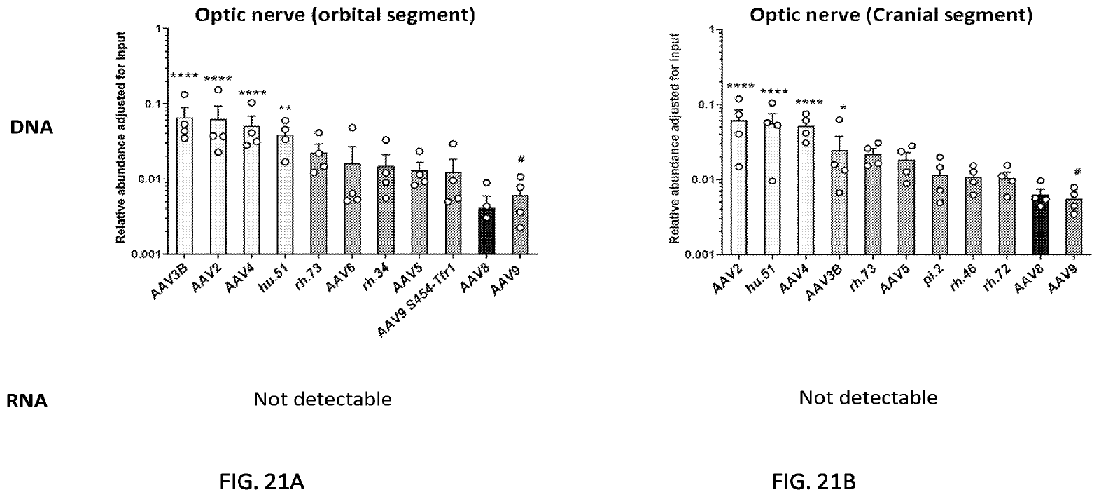

Results are presented for the top 9 capsids in relative abundance for vector DNA and transcribed transgene RNA relative to the abundance of AAV9. FIGS. 18A-18C represent relative abundance (to AAV9) of rAAV DNA and RNA for the nine most abundant capsid and controls AAV8 and AAV9 in dissected cornea (FIG. 18A), iris (FIG. 18B) and lens (FIG. 18C) tissue. RNA was not detectable in the cornea and lens tissue samples. FIGS. 19A-19C depict relative abundance (to AAV9) of rAAV DNA and RNA expressed from transgene barcoded by capsid, for the nine most abundant capsid and controls AAV8 and AAV9 in ciliary body (FIG. 19A), Schlemm's canal (FIG. 19B) and trabecular meshwork (FIG. 19C) tissues. Although not included in the bar graph, AAV3B RNA was detected in the ciliary body tissue (ranked 47 out of 118 in abundance) and in trabecular meshwork (ranked 26 out of 118 in abundance). FIGS. 20A-20C depict relative abundance (to AAV9) of rAAV DNA and RNA for the nine most abundant capsid and controls AAV8 and AAV9 in retina (FIG. 20A). RPE-Choroid (FIG. 20B) and sclera (FIG. 20C). Although not on the bar graph. AAV3B DNA was detected in the sclera (ranked 37 out of 118 in abundance). Finally, FIGS. 21A and 21B show the relative abundance (to AAV9) of rAAV DNA and RNA for the nine most abundant capsid and controls AAV8 and AAV9 in optic nerve (orbital segment) (FIG. 21A) or optical nerve (cranial segment) (FIG. 20B). RNA transcribed from transgene not detectable in the optic nerve samples either the orbital or cranial segment.

Relative RNA abundance, compared to AAV8 or AAV9 capsids, in the tissue is summarized in Table 8 below.

TABLE 8

| NHP ocular | Reference | Top performing capsids (fold of |
|---|---|---|
| tissue | capsid | RNA relative abundance) |
| Iris | AAV8 | AAV3B (29X); AAVrh.73 (13X) |
| | AAV9 | AAV3B (137X); AAVrh.73 (62X) |
| Ciliary body | AAV8 | AAVhu.26 (4X) |
| | AAV9 | AAVhu.26 (3X) |
| Trabecular | AAV8 | AAV1 (3X); AAVhu.26 (2X); AAV7 |
| meshwork | | (2X) |
| | AAV9 | AAV1 (4X); AAVhu.26 (3X); AAV7 |
| | | (3X) |
| Retina | AAV8 | AAV3B (489X) |
| | AAV9 | AAV3B (2479X) |
| RPE-Choroid | AAV8 | AAV3B (123X) |
| | AAV9 | AAV3B (481X) |
| Sclera | AAV8 | AAV1 (9X); AAV3B (7X) |
| | AAV9 | AAV1 (8X); AAV3B (6X) |

### 6.18. Example 17—Comparison of Biodistribution of Vectors in an rAAV Vector Pool in Cynomolgus Monkeys and Mice after IVT Injection

This example compares the biodistribution of an rAAV vector pool injected intravitreally in cynomolgus monkeys, as described in Example 16, infra, and mice as described in Example 13. In this example, 2 groups of 5 C57BL/6 mice were administered pooled vectors bilateral in each eye as detailed in Table 9 below and then sacrificed 3 weeks after administration. Tissues from one eye were collected and stored in RNAlater for RNA assays while tissues from the other eye were frozen for DNA analysis.

TABLE 9

| | Animal | Test | Dose | Endotoxin | Dose | Necropsy |
|---|---|---|---|---|---|---|
| Groups | number | Article | (GC/eye) | (EU/ml) | Volume | Date |
| 1 | 5 | AAV- | 1.2 × 10¹⁰ | 0.058 | 2 μL | 3 w |
| | | NAV- | | | per eye | |
| | | GFPbc- | | | | |
| | | IVT1 | | | | |
| 2 | 5 | AAV- | 1.6 × 10¹⁰ | <0.05 | 2 μL | 3 w |
| | | NAV- | | | per eye | |
| | | GFPbc- | | | | |
| | | IVT2 | | | | |

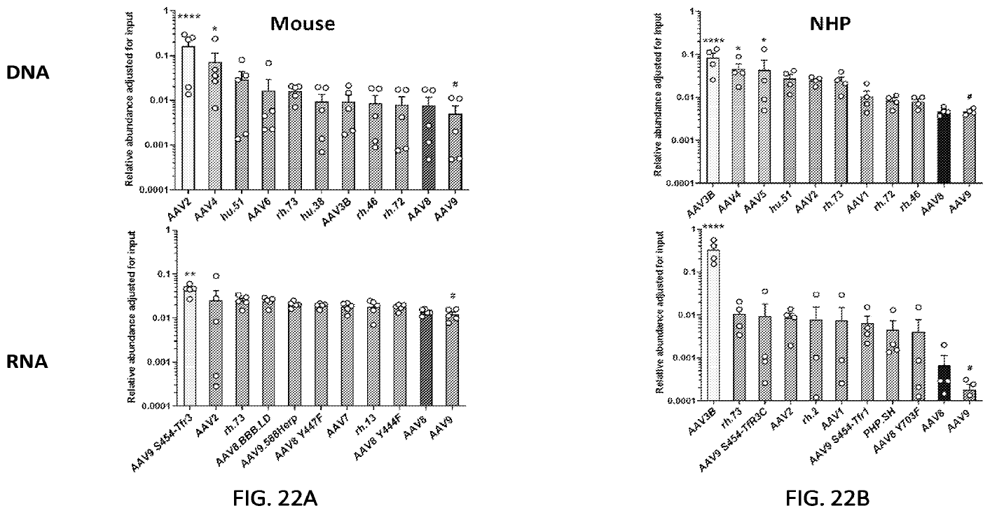

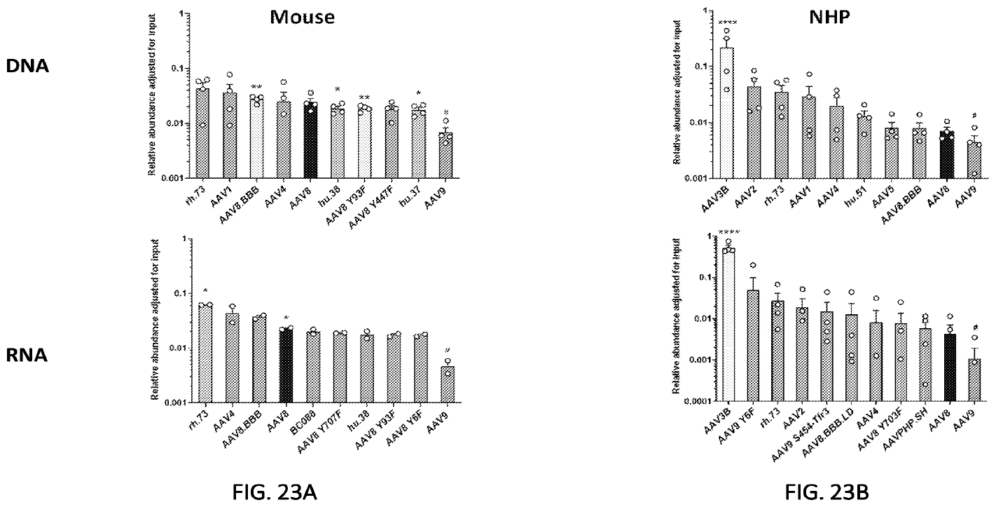

Biodistribution results showing the relative abundance of the DNA and RNA of rAAV of different capsids relative to AAV9 in retina tissue from mice (FIG. 22A) and NHP (FIG. 22B) and in RPE-choroid in mice (FIG. 23A) and NHP (FIG. 23B). Although not reflected in the bar graphs, AAV3B DNA was detected in mouse RPE-Choroid (ranked 73 out of 118 capsids in abundance relative to AAV9) and AAV3B RNA was detected in mouse retina (ranked 81 out of 118 capsids in abundance relative to AAV9) and in mouse RPE-Choroid (ranked 14 out of 118 capsids in abundance relative to AAV9).

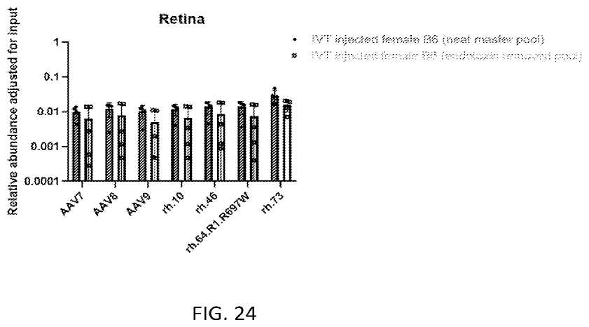

This study showed enrichment in retina and RPE-choroid tissue of AAV2 and AAV4 and also rh.73 in both mouse and NHP tissues when rAAV is administered via IVT administration. Relative abundance (DNA enrichment) of rh.73 in the pool of IVT injected female mice was also observed, as shown in FIG. 24.

### 6.19 Example 18: Capsid Biodistribution of a Single rAAV Vector Preparation of AAV3B in Cynomolgus Monkeys after Intravitreal (IVT) Injection

An rAAV vector preparation comprising a single AAV vector, AAV3B, expressing the GFP reporter gene from the universal CAG promoter (flanked by AAV2 ITRs) was administered to a group of 2 NHPs by IVT injection at a dose of 1.61E11 GC/eye (50 μL per eye injection volume). A control AAV2-variant (AAV2v) vector expressing GFP was also administered to a group of 2 NHPs by IVT injection at a dose of 1.61E11 GC/eye (50 μL per eye injection volume). The study followed a protocol analogous to that described in previous Examples, e.g. Examples 14, 15 and 16, whereas biodistribution of AAV3B or control vector DNA and RNA in various ocular tissues, as well as several peripheral tissues, will be assessed after sacrifice 3 weeks following the vector administration.

Ophthalmological examinations were performed prior to dose administration and on intermittent days following dose administration, e.g. examination by slit-lamp biomicroscopy, indirect ophthalmoscopy and IOP measurement. At 3 week sacrifice, ocular tissues, as well as optic nerve, were dissected and extracted (see FIGS. 16A and B for eye anatomy). Peripheral blood mononuclear cells (PBMCs), liver, brain, and lacrimal glands were also extracted. Tissues from the right eye were harvested and samples were collected in tubes with RNAlater (per manufacturer's instructions) and flash frozen at −80° C. until qPCR can be performed. Biodistribution of AAV3B capsid and transgene expression will be analyzed in the tissues of the left eye of each subject by RT-qPCR methods. Tissues of the right eye of each subject were enucleated, collected in 4% paraformaldehyde and processed to paraffin block and will be assessed by immunohistochemistry (IHC) for GFP expression, as well as hematoxylin and eosin (H&E) staining for histopathology analysis.

### 6.20 Example 19: Capsid Biodistribution of an rAAV Vector Pool in Cynomolgus Monkeys after Suprachoroidal Space (SCS) Injection

Pooled barcoded vectors were administered to NHPs by suprachoroidal injection. The pooled mixture consists of 118 different AAV capsids, including natural isolates and engineered AAVs, as described herein, expressing the GFP reporter gene from the universal CAG promoter. The suprachoroidal study followed a protocol analogous to that described in Examples 14, 15 and 16, infra, except, the vector pools were administered to 2 adult cynomolgus monkeys in both eyes (bilaterally) by SCS at a dose of 7.2E11 GC/eye. Prior to the suprachoroidal injections, animals were anesthetized with ketamine and dexmedetomidine. The AAV library (pool) was delivered to the suprachoroidal space (SCS) of each eye via a single SCS injection of 100 μL.

Ophthalmological examinations were performed prior to dose administration and on intermittent days, e.g. examination by slit-lamp biomicroscopy, indirect ophthalmoscopy and IOP measurement. At 3 week sacrifice, tissues were harvested and samples were collected in tubes with RNAlater (per manufacturer's instructions) and flash frozen at −80° C. until DNA and RNA analysis (biodistribution of each vector in the pool) can be performed by NGS in ocular tissues including aqueous humor, vitreous humor, choroid-retinal pigment epithelium (RPE), cornea, Iris-ciliary body, lens, optic nerve, retina and sclera.

### 6.21. Capsid Amino Acid Sequences

Table 10 provides the amino acid sequences of certain engineered capsid proteins and unengineered capsid proteins described and/or used in studies described herein. Heterologous peptides and amino acid substitutions are indicated in gray shading.

TABLE 10
Capsid Amino Acid Sequences

| Capsid | Insert or | | |
|---|---|---|---|
| Name | Substitution | Amino Acid Sequence | |
| PHP.S | QAVRTS | 1 | MAADGYLPDW LEDNLSEGIR EWWALKPGAP QPKANQQHQD NARGLVLPGY KYSGPGNGLD |
| (California | L (SEQ | 61 | KGEPVNAADA AALEHDKAYD CQLKAGDNPY LKYNHADAEF QERLKEDTSF GGNLGRAVFQ |
| Institute | ID | 121 | AKKRLLEPLG LVEEAAKTAP GKKRPVEQSP QEPDSSAGIG KSGAQPAKKR LNFGQTGDTE |
| of | NO: 16) | 181 | SVPDPQPIGE PPAAPSGVGS ITMASGGGAP VADNNEGADG VGSSSGNWHC DSQWLGDRVI |
| Technology- | (588_589) | 241 | TTSTRTWALP TYNNHLYKQI SNSTSGGSSN DNAYFGYSTP WGYFDFNRFH CHFSPRDWQR |
| Chan et | | 301 | LINNNWGFRP KRLNFKLFNI QVKEVTDNNG VKTIANNLTS TVQVFTDSDY QLPYVLGSAH |
| al 2017) | | 361 | EGCLPPFPAD VFMIPQYGYL TLNDGSQAVG RSSFYCLEYF PSQMLRTGNN FQFSYEFENV |
| | | 421 | PFHSSYAHSQ SLDRLMNPLI DQYLYYLSRT INGSGQNQQT LKFSVAGPSN MAVQGRNYIP |
| | | 481 | GPSYRQQRVS TTVTQNNNSE FAWPGASSWA LNGRNSLMNP GPAMASHKEG EDRFFPLSGS |
| | | 541 | LIFGKQGTGR DNVDADKVMI TNEEEIKTTN PVATESYGQV ATNHQSAQQA VRTSLAQAQT |
| | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 22) |
| PHP.SH | QAVRTS | 1 | MAADGYLPDW LEDNLSEGIR EWWALKPGAP QPKANQQHQD NARGLVLPGY KY_GPGNGLD |
| | H (SEQ | 61 | KGEPVNAADA AALEHDKAYD CQLKAGDNPY LKYNHADAEF QERLKEDTSF GGNLGRAVFQ |
| | ID | 121 | AKKRLLEPLG LVEEAAKTAP GKKRPVEQSP QEPDSSAGIG KSGAQPAKKR LNFGQTGDTE |
| | NO: 17) | 181 | SVPDPQPIGE PPAAPSGVGS ITMASGGGAP VADNNEGADG VGSSSGNWHC DSQWLGDRVI |
| | (588_589) | 241 | TTSTRTWALP TYNNHLYKQI SNSTSGGSSN DNAYFGYSTP WGYFDFNRFH CHFSPRDWQR |
| | | 301 | LINNNWGFRP KRLNFKLFNI QVKEVTDNNG VKTIANNLTS TVQVFTDSDY QLPYVLGSAH |
| | | 361 | EGCLPPFPAD VFMIPQYGYL TLNDGSQAVG RSSFYCLEYF PSQMLRTGNN FQFSYEFENV |
| | | 421 | PFHSSYAHSQ SLDRLMNPLI DQYLYYLSRT INGSGQNQQT LKFSVAGPSN MAVQGRNYIP |
| | | 481 | GPSYRQQRVS TTVTQNNNSE FAWPGASSWA LNGRNSLMNP GPAMASHKEG EDRFFPLSGS |
| | | 541 | LIFGKQGTGR DNVDADKVMI TNEEEIKTTN PVATESYGQV ATNHQSAQQA VRTSHAQAQT |
| | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 23) |
| PHP.B | TLAVPF | 1 | MAADGYLPDW LEDNLSEGIR EWWALKPGAP QPKANQQHQD NARGLVLPGY KY_GPGNGLD |
| (California | K (SEQ | 61 | KGEPVNAADA AALEHDKAYD CQLKAGDNPY LKYNHADAEF QERLKEDTSF GGNLGRAVFQ |
| Institute | | 121 | AKKRLLEPLG LVEEAAKTAP GKKRPVEQSP QEPDSSAGIG KSGAQPAKKR LNFGQTGDTE |
| of | ID | 181 | SVPDPQPIGE PPAAPSGVGS ITMASGGGAP VADNNEGADG VGSSSGNWHC DSQWLGDRVI |
| Technology | NO:20) | 241 | TTSTRTWALP TYNNHLYKQI SNSTSGGSSN DNAYFGYSTP WGYFDFNRFH CHFSPRDWQR |
| GenBank | (588_589) | 301 | LINNNWGFRP KRLNFKLFNI CVKEVTDNNG VKTIANNLTS TVQVFTDSDY QLPYVLGSAH |
| entry: | | 361 | EGCLPPFPAD VFMIPQYGYL TLNDGSQAVG RSSFYCLEYF PSQMLRTGNN FQFSYEFENV |
| ALU85156.1- | | 421 | PFHSSYAHSQ SLDRLMNPLI DQYLYYLSRT INGSGQNQQT LKFSVAGPSN MAVQGRNYIP |
| Deverman | | 481 | GPSYRQQRVS TTVTQNNNSE FAWPGASSWA LNGRNSLMNP GPAMASHKEG EDRFFPLSGS |
| et al | | 541 | LIFGKQGTGR DNVDADKVMI TNEEEIKTTN PVATESYGQV ATNHQSAQTL AVPFKAQAQT |
| 2016) | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 24) |
| PHP.eB | TLAVPF | 1 | MAADGYLPDW LEDNLSEGIR EWWALKPGAP QPKANQQHQD NARGLVLPGY KYSGPGNGLD |
| (California | K (SEQ | 61 | KGEPVNAADA AALEHDKAYD CQLKAGDNPY LKYNHADAEF QERLKEDTSF GGNLGRAVFQ |
| Institute | ID | 121 | AKKRLLEPLG LVEEAAKTAP GKKRPVEQSP QEPDSSAGIG KSGAQPAKKR LNFGQTGDTE |
| of | NO: 20) | 181 | SVPDPQPIGE PPAAPSGVGS ITMASGGGAP VADNNEGADG VGSSSGNWHC DSQWLGDRVI |
| Technology- | (588_589) | 241 | TTSTRTWALP TYNNHLYKQI SNSTSGGSSN DNAYFGYSTP WGYFDENRFH CHFSPRDWQR |
| Chan et | | 301 | LINNNWGFRP KRLNFKLFNI QVKEVTDNNG VKTIANNLTS TVQVFTDSDY QLPYVLGSAH |
| al 2017) | | 361 | EGCLPPFPAD VFMIPQYGYL TLNDGSQAVG RSSFYCLEYF PSQMLRTGNN FQFSYEFENV |
| | | 421 | PFHSSYAHSQ SLDRLMNPLI DQYLYYLSRT INGSGQNQQT LKFSVAGPSN MAVQGRNYIP |
| | | 481 | GPSYRQQRVS TTVTQNNNSE FAWPGASSWA LNGRNSLMNP GPAMASHKEG EDRFFPLSGS |
| | | 541 | LIFGKQGTGR DNVDADKVMI TNEEEIKTTN PVATESYGQV ATNHQSDGTL AVPFKAQAQT |
| | | 601 | GWVQNQGILP GMVWQDRDVY LQGPIWAKIP HTDGNFHPSP LMGGFGMKHP PPQILIKNTP |
| | | 661 | VPADPPTAFN KDKLNSFITQ YSTGQVSVEI EWELQKENSK RWNPEIQYTS NYYKSNNVEF |
| | | 721 | AVNTEGVYSE PRPIGTRYLT RNL (SEQ ID NO: 25) |

| | | | |
|---|---|---|---|
| AAV8. | A269S | MAADGYLPDW LEDNLSEGIR EWWALKPGAP KPKANQQKQD DGRGLVLPGY KYLGPFNGLD | 60 |
| BBB | | KGEPVNAADA AALEHDKAYD QQLQAGDNPY LRYNHADAEF QERLQEDTSF GGNLGRAVFQ | 120 |
| | | AKKRVLEPLG LVEEGAKTAP GKKRPVEPSP QRSPDSSTGI GKKGQQPARK RLNFGQTGDS | 180 |
| | | ESVPDPQPLG EPPAAPSGVG PNTMAAGGGA PMADNNEGAD GVGSSSGNWH CDSTWLGDRV | 240 |
| | | ITTSTRTWAL PTYNNHLYKQ ISNGTSGGST NDNTYFGYST PWGYFDENRF HCHFSPRDWQ | 300 |
| | | RLINNNWGFR PKRLSFKLEN IQVKEVTQNE GTKTIANNLT STIQVFTDSE YQLPYVIGSA | 360 |
| | | HQGCLPPFPA DVFMIPQYGY LTINNGSQAV GRSSFYCLEY FPSQMLRTGN NFCFTYTFED | 420 |
| | | VPFHSSYAHS QSLDRLMNPL IDQYLYYLSR TQTTGGTANT QTLGFSQGGP NTMANQAKNW | 480 |
| | | LPGPCYRQQR VSTTTGQNNN SNFAWTAGTK YHINGRNSLA NPGIAMATHK DDEERFFPSN | 540 |
| | | GILIFGKQNA ARDNADYSDV MLTSEEEIKT TNPVATEEYG IVADNLQQQN TAPQIGTVNS | 600 |
| | | QGALPGMVWQ NRDVYLQGPI WAKIPHTDGN FHPSPLMGGF GLKHPPPQIL IKNTPVPADP | 660 |
| | | PTTFNQSKLN SFITQYSTGQ VSVEIEWELQ KENSKRWNPE IQYTSNYYKS TSVDFAVNTE | 720 |
| | | GVYSEPRPIG TRYLTRNL (SEQ ID NQ: 26) | |
| AAV8. | A269S, | MAADGYLPDW LEDNLSEGIR EWWALKPGAP KPKANQQKQD DGRGLVLPGY KYLGPFNGLD | 60 |
| BBB.LD | 498_NNN/ | KGEPVNAADA AALEHDKAYD QQLQAGDNPY LRYNHADAEF QERLQEDTSF GGNLGRAVFQ | 120 |
| | AAA_500 | AKKRVLEPLG LVEEGAKTAP GKKRPVEPSP QRSPDSSTGI GKKGQQPARK RLNFGQTGDS | 180 |
| | | ESVPDPQPLG EPPAAPSGVG PNTMAAGGGA PMADNNEGAD GVGSSSGNWH CDSTWLGDRV | 240 |
| | | ITTSTRTWAL PTYNNHLYKQ ISNGTSGGST NDNTYFGYST PWGYFDFNRF HCHFSPRDWQ | 300 |
| | | RLINNNWGFR PKRLSFKLEN IQVKEVTQNE GTKTIANNLT STIQVFTDSE YQLPYVLGSA | 360 |
| | | HQGCLPPFPA DVFMIPQYGY LTINNGSQAV GRSSFYCLEY FPSQMLRTGN NFCFTYTFED | 420 |
| | | VPFHSSYAHS QSLERLMNPL IDQYLYYLSR TQTTGGTANT QTLGFSQGGP NTMANQAKNW | 480 |
| | | LPGPCYRQQR VSTTTGQAAA SNFAWTAGTK YHINGRNSLA NPGIAMATHK DDEERFFPSN | 540 |
| | | GILIFGKQNA ARDNADYSDV MLTSEEEIKT TNPVATEEYG IVADNLQQQN TAPQIGTVNS | 600 |
| | | QGALPGMVWQ NRDVYLQGPI WAKIPHTDGN FHPSPLMGGF GLKHPPPQIL IKNTPVPADP | 660 |
| | | PTTFNQSKLN SFITQYSTGQ VSVEIEWELQ KENSKRWNPE IQYTSNYYKS TSVDFAVNTE | 720 |
| | | GVYSEPRPIG TRYLTRNL (SEQ ID NO: 27) | |
| AAV9. | S263G/ | MAADGYLPDW LEDNLSEGIR EWWALKPGAP QPKANQQHQD NARGLVLPGY KYLGPGNGLD | |
| BBB | S269T/ | KGEPVNAADA AALEHDKAYD QQLKAGDNPY LKYNHADAEF QERLKEDTSF GGNLGRAVFQ | 120 |
| | A273T | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSCWLGDRVI | 240 |
| | | TTSTRTWALP TYNNHLYKQI SNGTSGGSTN DNTYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TINDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE FAWPGASSWA LNGRNSLMNP GPAMASHKEG EDRFFPSSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 28) | |
| AAV9. | S263G/ | MAADGYLPDW LEDNLSEGIR EWWALKPGAP QPKANQQHQD NARGLVLPGY KYLGPGNGLD | 60 |
| BBB.LD | S269T/ | KGEPVNAADA AALEHDKAYD QQLKAGDNPY LKYNHADAEF QERLKEDTSF GGNLGRAVFQ | 120 |
| | A273T, | AKKRLLEPLG LVEEAAKTAP GKKRPVEQSP QEPDSSAGIG KSGAQPAKKR LNFGQTGDTE | 180 |
| | 496_NNN/ | SVPDPQPIGE PPAAPSGVGS LTMASGGGAP VADNNEGADG VGSSSGNWHC DSCWLGDRVI | 240 |
| | AAA_498 | TTSTRTWALP TYNNHLYKQI SNGTSGGSTN DNTYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FAWPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 29) | |
| AAVrh. | 498_NNN/ | MAADGYLPDW LEDNLSEGIR EWWDIKPGAP KPKANQQKQD DGRGLVLPGY | 50 |
| 10.LD | AAA_500 | KYLGPFNGLD KGEPVNAADA AALEHDKAYD QQLKAGDNPY LRYNHADAEF | 100 |
| | | QERLQEDTSF GGNLGRAVFQ AKKRVLEPLG LVEEGAKTAP GKKRPVEPSP | 150 |
| | | QRSPDSSTGI GKKGQQPAKK RLNFGQTGDS ESVPDPQPIG EPPAGPSGLG | 200 |
| | | SGTMAAGGGA PMADNNEGAD GVGSSSGNWH CDSTWLGDRV ITTSTRTWAL | 250 |
| | | PTYNNHLYKQ ISNGTSGGST NDNTYFGYST PWGYFDFNRF HCHFSPRDWQ | 300 |
| | | RLINNNWGFR PKRINFKLEN IQVKEVTQNE GTKTIANNLT STIQVFTDSE | 350 |
| | | YQLPYVLGSA HQGCLPPFPA DVEMIPQYGY LTLNNGSQAV GRSSFYCLEY | 400 |
| | | FPSQMLRTGN NFEFSYQFED VPFHSSYAHS QSLDRIMNPL IDQYLYYLSR | 450 |
| | | TQSTGGTAGT QQLIFSQAGP NNMSAQAKNW LPGPCYRQQR VSTTLSQAAA | 500 |
| | | SNFAWTGATK YHLNGRDSLV NPGVAMATHK DDEERFFPSS GVLMFGKQGA | 550 |
| | | GKDNVDYSSV MLTSEEEIKT TNPVATEQYG VVADNLQQQN AAPIVGAVNS | 600 |
| | | QGALPGMVWQ NRDVYLQGPI WAKIPHTDGN FHPSPLMGGF GLKHPPPQIL | 650 |
| | | IKNTPVPADP PTTFSQAKLA SFITCYSTGQ VSVEIEWELQ KENSKRWNPE | 700 |
| | | IQYTSNYYKS TNVDFAVNTD GTYSEPRPIG TRYLTRNL (SEQ ID NO: 30) | |
| AAV9. | 498_NNN/ | MAADGYLPDW LEDNLSEGIR EWWALKPGAP QPKANQQHQD NARGLVLPGY KYLGPGNGLD | 60 |
| 496NNN/ | AAA_500 | KGEPVNAADA AALEHDKAYD QQLKAGDNPY LKYNHADAEF QERLKEDTSF GGNLGRAVFQ | 120 |
| AAA498 | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSCWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FAWPGASSWA LNGRNSLMNP GPAMASHKEG EDRFFPSSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 31) | |
| AAV9. | 496NNN/ | MAADGYLPDW LEDNLSEGIR EWWALKPGAP QPKANQQHQD NARGLVLPGY KYLGPGNGLD | 60 |
| 496NNN/ | AAA498, | KGEPVNAADA AALEHDKAYD QQLKAGDNPY LKYNHADAEF QERLKEDTSF GGNLGRAVFQ | 120 |
| AAA498. | W503R | AKKRLLEPLG LVEEAAKTAP GKKRPVEQSP QEPDSSAGIG KSGAQPAKKR LNFGQTGDTE | 180 |
| W503R | | SVPDPQPIGE PPAAPSGVGS LTMASGGGAP VADNNEGADG VGSSSGNWHC DSCWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VEMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQAAASE FARPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 32) | |
| AAV9 | W503R | MAADGYLPDW LEDNLSEGIR EWWALKPGAP QPKANQQHQD NARGLVLPGY KYLGPGNGLD | 60 |
| W503R | | KGEPVNAADA AALEHDKAYD QQLKAGDNPY LKYNHADAEF QERLKEDTSF GGNLGRAVFQ | 120 |
| | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSCWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSE FARPGASSWA LNGRNSLMNP GPAMASHKEG EDRFFPLSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 33) | |
| AAV9 | Q474A | MAADGYLPDW LEDNLSEGIR EWWALKPGAP QPKANQQHQD NARGLVLPGY KYLGPGNGLD | 60 |
| Q474A | | KGEPVNAADA AALEHDKAYD QQLKAGDNPY LKYNHADAEF QERLKEDTSF GGNLGRAVFQ | 120 |
| | | AKKRLLEPLG LVEEAAKTAP GKKRPVEQSP QEPDSSAGIG KSGAQPAKKR LNFGQTGDTE | 180 |
| | | SVPDPQPIGE PPAAPSGVGS LTMASGGGAP VADNNEGADG VGSSSGNWHC DSCWLGDRVI | 240 |
| | | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVAGRNYIP | 480 |
| | | GPSYRQQRVS TTVTQNNNSE FAWPGASSWA LNGRNSLMNP GPAMASHKEG EDRFFPUSGS | 540 |
| | | LIFGKQGTGR DNVDADKVMI TNEEEIKTTN PVATESYGQV ATNHQSAQAQ AQTGWVQNQG | 600 |
| | | ILPGMVWQDR DVYLQGPIWA KIPHTDGNFH PSPLMGGFGM KHPPPQILIK NTPVPADPPT | 660 |
| | | AFNKDKLNSF ITQYSTGQVS VEIEWELQKE NSKRWNPEIQ YTSNYYKSNN VEFAVNTEGV | 720 |
| | | YSEPRPIGTR YLTRNL (SEQ ID NO: 34) | |
| AAV9 | Bone1, | MAADGYLPDW LEDNLSEGIR EWWALKPGAP QPKANQQHQD NARGLVLPGY KYLGPGNGLD | 60 |
| S454-D8 | DDDDDD | KGEPVNAADA AALEHDKAYD QQLKAGDNPY LKYNHADAEF QERLKEDTSF GGNLGRAVFQ | 120 |
| | DD (SEQ | AKKRLLEPLG LVEEAAKTAP GKKRPVEQSP QEPDSSAGIG KSGAQPAKKR LNFGQTGDTE | 180 |
| | ID NO: 2) | SVPDPQPIGE PPAAPSGVGS LTMASGGGAP VADNNEGADG VGSSSGNWHC DSCWLGDRVI | 240 |
| | (454_455) | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TINDGSQAVG RSSFYCLEYF PSQMLRTGNN FQFSYEFENV | 420 |
| | | PFHSSYAHSQ SLDRLMNPLI DQYLYYLSKT INGSDDDDDD DDGQNQQTLKF SVAGPSNMAV | 481 |
| | | QGRNYIPGPS YRQCRVSTTV TQNNNSEFAW PGASSWALNG RNSLMNPGPA MASHKEGEDR | 541 |
| | | FFPLSGSLIF GKQGTGRDNV DADKVMITNE EEIKTTNPVA TESYGQVATN HQSAQAQAQT | 601 |
| | | GWVQNQGILP GMVWQDRDVY LQGPIWAKIP HTDGNFHPSP LMGGFGMKHP PPCILIKNTP | 661 |
| | | VPADPPTAFN KDKINSFITQ YSTGCVSVEI EWELQKENSK RWNPEIQYTS NYYKSNNVEF | 721 |
| | | AVNTEGVYSE PRPIGTRYLT RNL (SEQ ID NO: 35) | |
| AAV9 | Brain1, | MAADGYLPDW LEDNLSEGIR EWWALKPGAP QPKANQQHQD NARGLVLPGY KYLGPGNGLD | 60 |
| S454- | LSSRLD | KGEPVNAADA AALEHDKAYD QQLKAGDNPY LKYNHADAEF QERLKEDTSF GGNLGRAVFQ | 120 |
| Brain1 | A (SEQ ID | AKKRLLEPLG LVEEAAKTAP GKKRPVEQSP QEPDSSAGIG KSGAQPAKKR LNFGQTGDTE | 180 |
| | NO: 3) | SVPDPQPIGE PPAAPSGVGS LTMASGGGAP VADNNEGADG VGSSSGNWHC DSCWLGDRVI | 240 |
| | (454_455) | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSLSSRLD AGQNQQTLKF SVAGPSNMAV | 480 |
| | | QGRNYIPGPS YRQCRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPCILIKNTP | 660 |
| | | VPADPPTAFN KDKINSFITQ YSTGCVSVEI EWELQKENSK RWNPEIQYTS NYYKSNNVEF | 720 |
| | | AVNTEGVYSE PRPIGTRYLT RNL (SEQ ID NO: 36) | |
| AAV9 | Brain2/ | MAADGYLPDW LEDNLSEGIR EWWALKPGAP QPKANQQHQD NARGLVLPGY KYLGPGNGLD | 60 |
| S454- | Brain1C, | KGEPVNAADA AALEHDKAYD QQLKAGDNPY LKYNHADAEF QERLKEDTSF GGNLGRAVFQ | 120 |
| Brain2 | CLSSRL | AKKRLLEPLG LVEEAAKTAP GKKRPVEQSP QEPDSSAGIG KSGAQPAKKR LNFGQTGDTE | 180 |
| | DAC | SVPDPQPIGE PPAAPSGVGS LTMASGGGAP VADNNEGADG VGSSSGNWHC DSCWLGDRVI | 240 |
| | (SEQ ID | TTSTRTWALP TYNNHLYKQI SNSTSGGSSN DNAYFGYSTP WGYFDENRFH CHFSPRDWQR | 300 |
| | NO: 4) | LINNNWGFRP KRLNFKLFNI QVKEVTDNNG VKTIANNLTS TVQVFTDSDY QLPYVLGSAH | 360 |
| | (454_455) | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSCLSSRS DACGQNQQTL KFSVAGPSNM AV | 482 |
| | | QGRNYIPGPS YRQCRVSTTV TQNNNSEFAW PGASSWALNG RNSLMNPGPA MASHKEGEDR | 542 |
| | | FFPLSGSLIF GKQGTGRDNV DADKVMITNE EEIKTTNPVA TESYGQVATN HQSAQAQAQT | 602 |
| | | GWVQNQGILP GMVWQDRDVY LQGPIWAKIP HTDGNFHPSP LMGGFGMKHP PPCILIKNTP | 662 |
| | | VPADPPTAFN KDKINSFITQ YSTGCVSVEI EWELQKENSK RWNPEIQYTS NYYKSNNVEF | 722 |
| | | AVNTEGVYSE PRPIGTRYLT RNL (SEQ ID NO: 37) | |
| AAV9 | Kidney 1, | MAADGYLPDW LEDNLSEGIR EWWALKPGAP QPKANQQHQD NARGLVLPGY KYLGPGNGLD | 60 |
| S454- | LPVAS | KGEPVNAADA AALEHDKAYD QQLKAGDNPY LKYNHADAEF QERLKEDTSF GGNLGRAVFQ | 120 |
| Kidney1 | (SEQ ID | AKKRLLEPLG LVEEAAKTAP GKKRPVEQSP QEPDSSAGIG KSGAQPAKKR LNFGQTGDTE | 180 |
| | NO: 6) | SVPDPQPIGE PPAAPSGVGS LTMASGGGAP VADNNEGADG VGSSSGNWHC DSCWLGDRVI | 240 |
| | (454_455) | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSLPVASG QNQQTLKFSV AGPSNMAV | 478 |
| | | QGRNYIPGPS YRQCRVSTTV TQNNNSEFAW PGASSWALNG RNSLMNPGPA MASHKEGEDR | 538 |
| | | FFPLSGSLIF GKQGTGRDNV DADKVMITNE EEIKTTNPVA TESYGQVATN HQSAQAQAQT | 598 |
| | | GWVQNQGILP GMVWQDRDVY LQGPIWAKIP HTDGNFHPSP LMGGFGMKHP PPCILIKNTP | 658 |
| | | VPADPPTAFN KDKINSFITQ YSTGQVSVEI EWELQKENSK RWNPEIQYTS NYYKSNNVEF | 718 |
| | | AVNTEGVYSE PRPIGTRYLT RNL (SEQ ID NO: 38) | |
| AAV9 | Kidney2/ | MAADGYLPDW LEDNLSEGIR EWWALKPGAP QPKANQQHQD NARGLVLPGY KYLGPGNGLD | 60 |
| S454- | Kidney 1C, | KGEPVNAADA AALEHDKAYD QQLKAGDNPY LKYNHADAEF QERLKEDTSF GGNLGRAVFQ | 120 |
| Kidney2 | CLPVAS | AKKRLLEPLG LVEEAAKTAP GKKRPVEQSP QEPDSSAGIG KSGAQPAKKR LNFGQTGDTE | 180 |
| | C (SEQ | SVPDPQPIGE PPAAPSGVGS LTMASGGGAP VADNNEGADG VGSSSGNWHC DSCWLGDRVI | 240 |
| | ID NO: 5) | TTSTRTWALP TYNNHLYKQI SNSTSGGSSN DNAYFGYSTP WGYFDENRFH CHFSPRDWQR | 300 |
| | (454_455) | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSCLPVAS CGQNQQTLKF SVAGPSNMAV | 480 |
| | | QGRNYIPGPS YRQCRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPCILIKNTP | 660 |
| | | VPADPPTAFN KDKINSFITQ YSTGQVSVEI EWELQKENSK RWNPEIQYTS NYYKSNNVEF | 720 |
| | | AVNTEGVYSE PRPIGTRYLT RNL (SEQ ID NO: 39) | |
| AAV9 | Muscle1, | MAADGYLPDW LEDNLSEGIR EWWALKPGAP QPKANQQHQD NARGLVLPGY KYLGPGNGLD | 60 |
| S454- | ASSLNIA | KGEPVNAADA AALEHDKAYD QQLKAGDNPY LKYNHADAEF QERLKEDTSF GGNLGRAVFQ | 120 |
| Muscle1 | (SEQ ID | AKKRLLEPLG LVEEAAKTAP GKKRPVEQSP QEPDSSAGIG KSGAQPAKKR LNFGQTGDTE | 180 |
| | NO: 7) | SVPDPQPIGE PPAAPSGVGS LTMASGGGAP VADNNEGADG VGSSSGNWHC DSCWLGDRVI | 240 |
| | (454_455) | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TINDGSQAVG RSSFYCLEYF PSQMLRTGNN FQFSYEFENV | 420 |
| | | PFHSSYAHSQ SLDRLMNPLI DQYLYYLSKT INGSASSINI AGQNQQTLKF SVAGPSNMAV | 480 |
| | | QGRNYIPGPS YRQCRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPCILIKNT | 660 |
| | | VPADPPTAFN KDKINSFITQ YSTGQVSVEI EWELQKENSK RWNPEIQYTS NYYKSNNVEF | 720 |
| | | AVNTEGVYSE PRPIGTRYLT RNL (SEQ ID NO: 40) | |
| AAV9 | Tfr1, | MAADGYLPDW LEDNLSEGIR EWWALKPGAP QPKANQQHQD NARGLVLPGY KYLGPGNGLD | 60 |
| S454- | HAIYPR | KGEPVNAADA AALEHDKAYD QQLKAGDNPY LKYNHADAEF QERLKEDTSF GGNLGRAVEQ | 120 |
| Tfr1 | (SEQ ID | AKKRLLEPLG LVEEAAKTAP GKKRPVEQSP QEPDSSAGIG KSGAQPAKKR LNFGQTGDTE | 180 |
| | NO: 106) | SVPDPQPIGE PPAAPSGVGS LTMASGGGAP VADNNEGADG VGSSSGNWHC DSCWLGDRVI | 240 |
| | (454_455) | TTSTRTWALP TYNNHLYKQI SNSTSGGSSN DNAYFGYSTP WGYFDFNRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSHAIYPR HGQNQQTLKF SVAGPSNMAV | 480 |
| | | QGRNYIPGPS YRQCRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPCILIKNTP | 660 |
| | | VPADPPTAFN KDKINSFITQ YSTGCVSVEI EWELQKENSK RWNPEIQYTS NYYKSNNVEF | 720 |
| | | AVNTEGVYSE PRPIGTRYLT RNL (SEQ ID NO: 41) | |
| AAV9 | Tfr3, | MAADGYLPDW LEDNLSEGIR EWWALKPGAP QPKANQQHQD NARGLVLPGY KYLGPGNGLD | 60 |
| S454- | RTIGPSV | KGEPVNAADA AALEHDKAYD QQLKAGDNPY LKYNHADAEF QERLKEDTSF GGNLGRAVFQ | 120 |
| Tfr3 | (SEQ ID | AKKRLLEPLG LVEEAAKTAP GKKRPVEQSP QEPDSSAGIG KSGAQPAKKR LNFGQTGDTE | 180 |
| | NO: 12) | SVPDPQPIGE PPAAPSGVGS LTMASGGGAP VADNNEGADG VGSSSGNWHC DSCWLGDRVI | 240 |
| | (454_455) | TTSTRTWALP TYNNHLYKQI SNSTSGGSSN DNAYFGYSTP WGYFDENRFH CHFSPRDWQR | 300 |
| | | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSRTIGPS VGQNQQTLKF SVAGPSNMAV | 480 |
| | | QGRNYIPGPS YRQCRVSTTV TQNNNSEFAW PGASSWALNG RNSLMNPGPA MASHKEGEDR | 540 |
| | | FFPLSGSLIF GKQGTGRDNV DADKVMITNE EEIKTTNPVA TESYGQVATN HQSAQAQAQT | 600 |
| | | GWVQNQGILP GMVWQDRDVY LQGPIWAKIP HTDGNFHPSP LMGGFGMKHP PPCILIKNTP | 660 |
| | | VPADPPTAFN KDKINSFITQ YSTGQVSVEI EWELQKENSK RWNPEIQYTS NYYKSNNVEF | 720 |
| | | AVNTEGVYSE PRPIGTRYLT RNL (SEQ ID NO: 42) | |
| AAV9 | Tfr4, | MAADGYLPDW LEDNLSEGIR EWWALKPGAP QPKANQQHQD NARGLVLPGY KYLGPGNGLD | 60 |
| S454- | CRTIGPS | KGEPVNAADA AALEHDKAYD QQLKAGDNPY LKYNHADAEF QERLKEDTSF GGNLGRAVFQ | 120 |
| Tfr4 | VC (SEQ | AKKRLLEPLG LVEEAAKTAP GKKRPVEQSP QEPDSSAGIG KSGAQPAKKR LNFGQTGDTE | 180 |
| (AAV9 | ID | SVPDPQPIGE PPAAPSGVGS LTMASGGGAP VADNNEGADG VGSSSGNWHC DSCWLGDRVI | 240 |
| S454- | NO: 13) | TTSTRTWALP TYNNHLYKQI SNSTSGGSSN DNAYFGYSTP WGYFDFNRFH CHFSPRDWQR | 300 |
| TfR3C) | (454_455) | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSCRTIGP SVCGQNQQTL KFSVAGPSNMAV | 482 |
| | | QGRNYIPGPS YRQCRVSTTV TQNNNSEFAW PGASSWALNG RNSLMNPGPA MASHKEGEDR | 542 |
| | | FFPLSGSLIF GKQGTGRDNV DADKVMITNE EEIKTTNPVA TESYGQVATN HQSAQAQAQT | 602 |
| | | GWVQNQGILP GMVWQDRDVY LQGPIWAKIP HTDGNFHPSP LMGGFGMKHP PPCILIKNTP | 662 |
| | | VPADPPTAFN KDKINSFITQ YSTGQVSVEI EWELQKENSK RWNPEIQYTS NYYKSNNVEF | 722 |
| | | AVNTEGVYSE PRPIGTRYLT RNL (SEQ ID NO: 43) | |
| AAV9. | SITLVKS | MAADGYLPDW LEDNLSEGIR EWWALKPGAP QPKANQQHQD NARGLVLPGY KYLGPGNGLD | 60 |
| 588Ad | TQTV | KGEPVNAADA AALEHDKAYD QQLKAGDNPY LKYNHADAEF QERLKEDTSF GGNLGRAVFQ | 120 |
| (9 588 | (SEQ ID | AKKRLLEPLG LVEEAAKTAP GKKRPVEQSP QEPDSSAGIG KSGAQPAKKR LNFGQTGDTE | 180 |
| Ad) | NO: 14), | SVPDPQPIGE PPAAPSGVGS LTMASGGGAP VADNNEGADG VGSSSGNWHC DSCWLGDRVI | 240 |
| | DLC-AS1 | TTSTRTWALP TYNNHLYKQI SNSTSGGSSN DNAYFGYSTP WGYFDFNRFH CHFSPRDWQR | 300 |
| | (588_589) | LINNNWGFRP KRLNFKLFNI QVKEVTDNNG VKTIANNLTS TVQVFTDSDY QLPYVLGSAH | 360 |
| | | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |
| | | GPSYRQQRVS TTVTQNNNSI TLVKSTQTVS EFAWPGASSW ALNGRNSLMN PGPAMASHKE | 540 |
| | | GEDRFFPLSG SLIFGKQGTG RDNVDADKVM ITNEEEIKTT NPVATESYGQ VATNHQSAQA | 600 |
| | | QAQTGWVQNQ GILPGMVWQD RDVYLQGPIW AKIPHTDGNF HPSPLMGGFG MKHPPPQILI | 660 |
| | | KNTPVPADPP TAFNKDKLNS FITQYSTGQV SVEIEWELQK ENSKRWNPEI QYTSNYYKS | 720 |
| | | NVEFAVNTEG VYSEPRPIGT RYLTRNL (SEQ ID NO: 44) | |
| AAV9. | TILSRST | MAADGYLPDW LEDNLSEGIR EWWALKPGAP QPKANQQHQD NARGLVLPGY KYLGPGNGLD | 60 |
| 88 | QTG | KGEPVNAADA AALEHDKAYD QQLKAGDNPY LKYNHADAEF QERLKEDTSF GGNLGRAVFQ | 120 |
| 5Herp | (SEQ ID | AKKRLLEPLG LVEEAAKTAP GKKRPVEQSP QEPDSSAGIG KSGAQPAKKR LNFGQTGDTE | 180 |
| (9 588 | NO: 15), | SVPDPQPIGE PPAAPSGVGS LTMASGGGAP VADNNEGADG VGSSSGNWHC DSCWLGDRVI | 240 |
| Hep) | DLC- | TTSTRTWALP TYNNHLYKQI SNSTSGGSSN DNAYFGYSTP WGYFDFNRFH CHFSPRDWQR | 300 |
| | AS2, | LINNNWGFRP KRLNFKLFNI QVKEVTDNNG VKTIANNLTS TVQVFTDSDY QLPYVLGSAH | 360 |
| | 588_589 | EGCLPPFPAD VFMIPQYGYL TLNDGSQAVG RSSFYCLEYF PSQMLRTGNN FQFSYEFENV | 420 |
| | | PFHSSYAHSQ SLDRLMNPLI DQYLYYLSKT INGSGQNQQT LKFSVAGPSN MAVQGRNYIP | 480 |

| | |
|---|---|
| GPSYRQQRVS TTVTQNNNTI LSRSTQTGSE FAWPGASSWA LNGRNSLMNP GPAMASHKEG | 540 |
| EDRFFPLSGS LIFGKQGTGR DNVDADKVMI TNEEEIKTTN PVATESYGQV ATNHQSAQAQ | 600 |
| AQTGWVQNQG ILPGMVWQDR DVYLCGPIWA KIPHTDGNFH PSPLMGGFGM KHPPPQILIK | 660 |
| NTPVPADPPT AFNKDKLNSF ITQYSTGQVS VEIEWELQKE NSKRWNPEIQ YTSNYYKSNN | 720 |

| | | VEFAVNTEG VYSEPRPIGT RYLTRNL (SEQ ID NO: 45) | |
|---|---|---|---|
| AAVPH | SITLVKS | 1 | MAADGYLPDW LEDNLSEGIR EWWALKPGAP QPKANQQHQD NARGLVLPGY KYLGPGNGLD |
| PeB.VP2 | TQTV | 61 | KGEPVNAADA AALEHDKAYD CQLKAGDNPY LKYNHADAEF QERLKEDTSF GGNLGRAVFQ |
| Ad | (SEQ ID | 121 | AKKRLLEPLG LVEEAAKTTI LSRSTQTGAP GKKRPVEQSP QEPDSSAGIG KSGAQPAKKR |
| | NO: 14), | | LNFGQTGDTE |
| | DLC-AS1 | 191 | SVPDPQPIGE PPAAPSGVGS ITMASGGGAP VADNNEGADG VGSSSGNWHC DSQWLGDRVI |
| | (138_139) | 251 | TTSTRTWALP TYNNHLYKQI SNSTSGGSSN DNAYFGYSTP WGYFDFNRFH CHFSPRDWQR |
| | | 311 | LINNNWGFRP KRLNFKLFNI QVKEVTDNNG VKTIANNLTS TVQVFTDSDY QLPYVLGSAH |
| | | 371 | EGCLPPFPAD VFMIPQYGYL TLNDGSQAVG RSSFYCLEYF PSQMLRTGNN FQFSYEFENV |
| | | 431 | PFHSSYAHSQ SLDRLMNPLI DQYLYYLSRT INGSGQNQQT LKFSVAGPSN MAVQGRNYIP |
| | | 491 | GPSYRQQRVS TTVTQNNNSE FAWPGASSWA LNGRNSLMNP GPAMASHKEG EDRFFPLSGS |
| | | 551 | LIFGKQGTGR DNVDADKVMI TNEEEIKTTN PVATESYGQV ATNHQSDGTL AVPFKAQAQT |
| | | 611 | GWVQNQGILP GMVWQDRDVY LQGPIWAKIP HTDGNFHPSP LMGGFGMKHP PPQILIKNTP |
| | | 671 | VPADPPTAFN KDKLNSFITQ YSTGQVSVEI EWELQKENSK RWNPEIQYTS NYYKSNNVEF |
| | | 731 | AVNTEGVYSE PRPIGTRYLT RNL (SEQ ID NO: 46) |
| AAVPH | TILSRST | 1 | MAADGYLPDW LEDNLSEGIR EWWALKPGAP QPKANQQHQD NARGLVLPGY KYSGPGNGLD |
| PeB.VP2 | QTG | 61 | KGEPVNAADA AALEHDKAYD CQLKAGDNPY LKYNHADAEF QERLKEDTSF GGNLGRAVFQ |
| Herp | (SEQ ID | 121 | AKKRLLEPLG LVEEAAKTSI TLVKSTQTVA PGKKRPVEQS PQEPDSSAGI GKSGAQPAKK |
| | NO: 15), | | RLNFGQTGDT E |
| | DLC- | 192 | SVPDPQPIGE PPAAPSGVGS ITMASGGGAP VADNNEGADG VGSSSGNWHC DSQWLGDRVI |
| | AS2, | 252 | TTSTRTWALP TYNNHLYKQI SNSTSGGSSN DNAYFGYSTP WGYFDENRFH CHFSPRDWQR |
| | (138_139) | 312 | LINNNWGFRP KRLNFKLFNI QVKEVTDNNG VKTIANNLTS TVQVFTDSDY QLPYVLGSAH |
| | | 372 | EGCLPPFPAD VFMIPQYGYL TLNDGSQAVG RSSFYCLEYF PSQMLRTGNN FQFSYEFENV |
| | | 432 | PFHSSYAHSQ SLDRLMNPLI DQYLYYLSRT INGSGQNQQT LKFSVAGPSN MAVQGRNYIP |
| | | 492 | GPSYRQQRVS TTVTQNNNSE FAWPGASSWA LNGRNSLMNP GPAMASHKEG EDRFFPLSGS |
| | | 552 | LIFGKQGTGR DNVDADKVMI TNEEEIKTTN PVATESYGQV ATNHQSDGTL AVPFKAQAQT |
| | | 612 | GWVQNQGILP GMVWQDRDVY LQGPIWAKIP HTDGNFHPSP LMGGFGMKHP PPQILIKNTP |
| | | 672 | VPADPPTAFN KDKLNSFITQ YSTGQVSVEI EWELQKENSK RWNPEIQYTS NYYKSNNVEF |
| | | 732 | AVNTEGVYSE PRPIGTRYLT RNL (SEQ ID NO: 47) |

| | | |
|---|---|---|
| AAV.rh. | — | MAADGYLPDW LEDNLSEGIR EWWDLKPGAP KPKANQQKQD DGRGLVLPGY EYLGPENGLD |
| 34 | | KGEPVNAADA AALEHDKAYD QQLKAGDNPY LRYNHADAEF QERLQEDTSF GGNLGRAVFQ |
| | | AKKRVLEPLG LVEEGAKTAP GKKRPLESPQ EPDSSSGIGK KGKQPAKKRL NFEEDTGAGD |
| | | GPPEGSDTSA MSSDIEMRAA PGGNAVDAGQ GSDGVGNASG DWHCDSTWSE GKVTTTSTRT |
| | | WVLPTYNNHL YLRIGTTSNS NTYNGFSTPW GYFDFNRFHC HFSPRDWQRL INNNWGLRPK |
| | | AMRVKIFNIQ VKEVTTSNGE TTVANNLTST VQIFADSSYE LPYVMDAGQE GSIPPFPNDV |
| | | FMVPQYGYCG IVTGENQNQT DRNAFYCLEY FPSQMLRTGN NFETAYNFEK VPFHSMYAHS |
| | | QSLDGLMNPL LDQYLWHLQS TTSGETINQG NAATTFGKIR SGDFAFYRKN WLPGPCVKQQ |
| | | RFSKTASQNY KIPASGGNAL LKYDTHYTLN NRWSNIAPGP PMATAGPSDG DFSNAQLIFP |
| | | GPSVTGNTTT SANNLLFTSE EEIAATNPRD TDMFGQIADN NQNATTAPIT GNVTAMGVLP |
| | | GMVWQNRDIY YQGPIWAKIP HADGHFHPSP LIGGFGLKHP PPQIFIKNTP VPAYPATTFT |
| | | AARVDSFITQ YSTGQVAVQI EWEIEKERSK RWNPEVQFTS NCGNQSSMLW APDTTGKYTE |
| | | PRVIGSRYLT NHL (SEQ ID NO: 81) |
| AAV.hu. | — | MAADGYLPDW LEDTLSEGIR QWWKIKPGPP PPKPAERHKD DSRGLVLPGY KYLGPGNGLD |
| 31 | | KGEPVNAADA AALEHDKAYD QQLKAGDNPY LKYNHADAEF QERLKEDTSF GGNLGRAVFQ |
| | | AKKRLLEPLG LVEEAAKTAP GKKRPVEQSP QEPDSSAGIG KSGSQPAKKK LNFGQTGDTE |
| | | SVPDPQPIGE PPAAPSGVGS LTMASGGGAP VADNNEGADG VGSSSGNWHC DSCWLGDRVI |
| | | TTSTRTWALP TYNNHLYKQI SNSTSGGSSN DNAYFGYSTP WGYFDENRFH CHFSPRDWQR |
| | | LINNNWGFRP KRLNFKLFNI QVKEVTDNNG VKTIANNLTS TVQVFTDSDY QLPYVLGSAH |
| | | EGCLPPFPAD VFMIPQYGYL TINDGGQAVG RSSFYCLEYF PSQMLRTGNN FQFSYEFENV |
| | | PFHSSYAHSQ SLDRLMNPLI DQYLYYLSKT INGSGQNQQT LKFSVAGPSN MAVQGRNYIP |
| | | GPSYRQQRVS TTVTQNNNSE FAWPGASSWA LNGRNSLMNP GPAMASHKEG EDRFFPLSGS |
| | | LIFGKQGTGR DNVDADKVMI TNEEEIKTTN PVATESYGQV ATNHQSAQAQ AQTGWVQNQG |
| | | ILPGMVWQDR DVYLQGPIWA KIPHTDGNFH PSPLMGGFGM KHPPPQILIK NTPVPADPPT |
| | | AFNKDKLNSF ITQYSTGQVS VEIEWELQKE NSKRWNPEIQ YTSNYYKSNN VEFAVSTEGV |
| | | YSEPRPIGTR YLTRNL (SEQ ID NO: 82) |
| AAV.rh. | — | KAYDQQLKAG DNPYLRYNHA DAEFCERLQE DTSFGGNLGR AVFQAKKRVL EPIGLVETPA |
| 31 | | KTAPGKKRPV DSPDSTSGIG KKGQCPAKKR LNFGQTGDSE SVPDPQPIGE PPAGPSGLGS |
| | | GTMAAGGGAP MADNNEGADG VGNASGNWHC DSTWLGDRVI TTSTRTWALP TYNNHLYKQI |
| | | SSQSAGSTND NVYFGYSTPW GYFDFNRFHC HFSPRDWQRL INNNWGFRPK KLNFKLFNIQ |
| | | VKEVTTNDGV TTIANNLTST VQVFSDSEYQ LPYVLGSAHQ GCLPPFPADV FMIPQYGYLT |
| | | LNNGSQSVGR SSFYCLEYFP SQMLRTGNNF TFSYTFEDVP FHSSYAHSQS LDRLMNPLID |
| | | QYLYYLARTQ SNAGGTAGNR ELQFYQGGPT TMAEQAKNWL PGPCFRQQRV SKTLDQNNNS |
| | | NFAWTGATKY HLNXRNSLVN PGVAMATHKD DEERFFPSSG VLIFGKTGAA NKTTLENVLM |
| | | TNEEEIRPTN PVATEEYGIV SSNLCAASTA AQTQVVNNQG ALPGMVWQNR DVYLQGPIWA |
| | | KIPHTDGNFH PSPIMGGFGL KHPPPQILIK NTPVPANPPE VFTPAKFASF ITCYSTGQVS |
| | | VEIEWELQKE NSKRWNPEIQ YTSNEDKQTG VDFAVDSQGV YSEP (SEQ ID NO: 83) |
| AAV. | — | MAADGYLPDW LEDTLSEGIR QWWKIKPGPP PPKPAERHQD DSRGLVLPGY KYLGPENGLD |
| hu.12 | | KGEPVNEADA AALEHDKAYD RQLDSGDNPY LKYNHADAEF QERLKEDTSF GGNLGRAVFQ |
| | | AKKRVLEPLG LVEEPVKTAP GKKRPVEHSP VEPDSSSGTG KAGHQPARKR LNFGQTGDAD |
| | | SVPDPQPLGQ PPAAPTSLGS TTMATGSGAP MADNNEGADG VGNSSGNWHC DSCWLGDRVI |
| | | TTSTRTWALP TYNNHLYKQI SSQSGASNDN HYFGYSTPWG YFDFNRFHCH FSPRDWQRLI |
| | | NNNWGFRPKR LNFKLFNIQV KEVTQNDGTT TIANNLTSTV QVFTDSEYQL PYVLGSAHQG |
| | | CLPPFPADVF MVPCYGYLTL NNGSCAVGRP SFYCLEYFPS QMLRTGNNFT FSYTFEDVPF |
| | | HSSYAHSQSL DRLMNPLIDQ YLYYINRTQS NSGTLQQSRL LFSQAGPTSM SLCAKNWLPG |
| | | PCYRQQRLSK QANDNNNSNF PWTAATKYHL NGRDSLVNPG PAMASHKDDE EKFFPMHGTL |
| | | IFGKQGTNAN DADLEHVMIT DEEEIRTTNP VATEQYGNVS NNLQNSNTGP TTENVNHQGA |
| | | LPGMVWQDRD VYLCGPIWAK IPHTDGHFHP SPLMGGFGLK HPPPQIMIKN TPVPANPPTN |
| | | FSSAKFASFI TQYSTGQVSV EIEWELQKEN SKRWNPEIQY TSNYNKSVNV DFTVDTNGVY |
| | | SEPRPIGTRY LTRNL (SEQ ID NO: 84) |
| AAV.hu. | — | MAADGYLPDW LEDTLSEGIR QWWKIKPGPP PPKPAERHKD DSRGLVLPGY KYLGPFNGLD |
| 13 | | KGEPVNEADA AALEHDKAYD RQLDSGDNPY LKYNHADAEF QERLKEDTSF GGNLGRAVFQ |
| | | AKKRVLEPLG LVEEPVKTAP GKKRPVEHSP AEPDSSSGTG KAGQQPARKR LNFGQTGDAD |
| | | SVPDPQPLGQ PPAAPSGLGT NTMASGSGAP MADNNEGADG VGNSSGNWHC DSTWMGDRVI |
| | | TTSTRTWALP TYNNHLYKQI SSQSGASNDN HYFGYSTPWG YFDFNRFHCH FSPRDWQRLI |
| | | NNNWGFRPKR LNFKLFNIQV KEVTQNDGTT TIANNLTSTV QVFTDSEYQL PYVLGSAHQG |
| | | CLPPFPADVF MVPCYGYLTL NNGSCAVGRS SFYCLEYFPS QMLRTGNNFT FSYTFEDVPF |
| | | HSSYAHSQSL DRLMNPLIDQ YLYYLSRTNT PSGTTTQSRS QFSQAGASDI RDCSRNWLPG |
| | | PCYRQQRVSK TSADNNNSEY SWTGATKYHL NGRDSLVNPG PAMASHKDDE EKFFPQSGVL |
| | | IFGKQGSEKT NVDIEKVMIT DEEEIRTTNP VATEQYGSVS INLQGGNTQA ATADVNTQGV |
| | | LPGMVWQDRD VYLCGPIWAK IPHTDGHFHP SPLMGGFGLK HPPPQILIKN TPVPANPSTT |
| | | FSAAKFASFI TQYSTGQVSV EIEWELQKEN SKRWNPEIQY TSNYNKSVNV DFTVDTNGVY |
| | | SEPRPIGTRY LTRNL (SEQ ID NO: 85) |
| AAV.hu. | — | MAADGYLPDW LEDTLSEGIR QWWKIKPGPP PPKPAERHKD DSRGLVLPGY KYLGPENGLD |
| 21 | | KGEPVNEADA AALEHDKAYD RQLDSGDNPY LKYNHADAEF QERLKEDTSF GGNLGRAVFQ |
| | | AKKRILEPLG LVEEPVKTAP GKKRPVEHSP AEPDSSSGTG KAGQQPARKR LNFGQTGDAD |
| | | SVPDPRPLGQ PPAAPSGLGT NTMASGSGAP MADNNEGADG VGNSSGNWHC DSTWMGDRVI |
| | | TTSTRTWALP TYNNHLYKQI SSQSGASNDN HYFGYSTPWG YFDFNRFHCH FSPRDWQRLI |
| | | NNNWGFRPKR LSFKLFNIQV KEVTQNDGTT TIANNLTSTV QVFTDSEYQL PYVLGSAHQG |
| | | CLPPFPADVF MVPCYGYLTL NNGSCAVGRS SFYCLEYFPS QMLRTGNNFT FSYTFEDVPF |
| | | HSSYAHSQSL DRLMNPLIDQ YLYYLSRINT PSGTTTMSRS QFSQAGASDI RDCSRNWLPG |
| | | PCYRQQRVSK TAADNNNSDY SWTGATKYHL NGRDSLVNPG PAMASHKDDE EKYFPQSGVL |
| | | IFGKQDSGKT NVDIEKVMIT DEEEIRTTNP VATEQYGSVS INLQSGNTQA ATSDVNTQGV |
| | | LPGMVWQDRD VYLCGPIWAK IPHTDGHFHP SPLMGGFGLK HPPPQILIKN TPVPANPSTT |
| | | FSAAKFASFI TQYSTGQVSV EIEWELQKEN SKRWNPEIQY TSNYNKSVNV DFTVDTNGVY |
| | | SEPRPIGTRY LTRNL (SEQ ID NO: 77) |
| AAV.hu. | — | MAADGYLPDW LEDTLSEGIR QWWKIKPGPP PPKPAERHKD DSRGLVLPGY KYLGPENGLD |
| 26 | | KGEPVNEADA AALEHDKAYD RQLDSGDNPY LKYNHADAEF QERLKEDTSF GGNLGRAVFQ |
| | | AKKRILEPLG LVEEPVKTAP GKKRPVEHSP AEPDSSSGTG KAGQQPARKR LNFGQTGDAD |
| | | SVPDPQPLGQ PPAAPSGLGT NTMASGSGAP MADNNEGADG VGNSSGNWHC DSTWMGDRVI |
| | | TTSTRTWALP TYNNHLYKQI SSQSGASNDN HYFGYSTPWG YFDFNRFHCH FSPRDWQRLI |
| | | NNNWGFRPKR LSFKLFNIQV KEVTQNDGTT TIANNLTSTV QVFTDSEYQL PYVLGSAHQG |
| | | CLPPFPADVF MVPCYGYLTL NNGSCAVGRS SFYCLEYFPS QMLRTGNNFT FSYTFEDVPF |
| | | HSSYAHSQSL DRLMNPLIDQ YLYYLSRTNT PSGTTTMSRL QFSQAGASDI RDCSRNWLPG |
| | | PCYRQQRVSK TAADNNNSDY SWTGATKYHL NGRDSLVNPG PAMASHKDDE EKYFPQSGVL |
| | | IFGKQDSGKT NVDIEKVMIT DEEEIRTTNP VATEQYGSVS TNLQSGNTQA ATSDVNTQGV |
| | | LPGMVWQDRD VYLQGPIWAK IPHTDGHFHP SPLMGGFGLK HPPPQILIKN TPVPANPSTT |
| | | FSAAKFASFI TQYSTGQVSV EIEWELQKEN SKRWNPEIQY TSNYNKSVNV DFTVDTNGVY |
| | | SEPRPIGTRY LTRNL (SEQ ID NO: 79) |
| AAV.hu. | — | MAADGYLPDW LEDTLSEGIR QWWKIKPGPP PPKPAERHKD DSRGLVLPGY KYLGPENGLD |
| 53 | | KGEPVNEADA AALEHDKAYD RQLDSGDNPY LKYNHADAEF QERLKEDTSF GGNLGRAVFQ |
| | | AKKRVLEPLG LVEEPVKTAP GKKRPVEHSP AEPDSSSGTG KAGQQPARKR LNFGQTGDAD |
| | | SVPDPQPLRQ PPAAPTSLGS TTMATGSGAP MADNNEGADG VGNSSGNWHC DSCWLGDRVI |
| | | TTSTRTWALP TYNNHLYKQI SSQSGASNDN HYFGYSTPWG YFDFNRFHCH FSPRDWQRLI |
| | | NNNWGFRPKR LNFKLFNIQV KEVTQNDGTT TIANNLTSTV QVFTDSEYQL PYVLGSAHQG |
| | | CLPPFPADVF MVPCYGYLTL NNGSCAVGRS SFYCLEYFPS QMLRTGNNFQ FSYTFEDVPF |
| | | HSSYAHSQSL DRLMNPLIDQ YLYYINRTQT ASGTQQSRLS FSQAGPTSMS LQAKNWLPGP |
| | | CYRQQRLSKQ ANDNNNSNFP WTGATKYYLN GRDSLVNPGP AMASHKDDEE KFFPMHGTLI |
| | | FGKEGTNATN AELENVMITD EEEIRTTNPV ATEQYGYVSN NLQNSNTAAS TETVNHQGAL |
| | | PGMVWQDRDV YLQGPIWAKI PHTDGHFHPS PLMGGFGLKH PPPQIMIKNT PVPANPPTNF |
| | | SSAKFASFIT QYSTGQVSVE IEWELQKENS KRWNPEIQYT SNYNKSVNVD FTVDINGVYS |
| | | EPRPIGTRYL TRNI (SEQ ID NO: 80) |
| AAV.hu. | — | MAADGYLPDW LEDTLSEGIR QWWKIKPGPP PPKPAERHKD DSRGLVLPGY KYLGPFNGLD |
| 56 | | KGEPVNEADA AALEHDKAYD RQLDSGDNPY LKYNHADAEF QERLKEDTSF GGNLGRAVFQ |
| | | AKKRVLEPLG LVEEPVKTAP GKKRPVEHSP VEPDSSSGTG KAGNQPARKR LNFGQTGDAD |
| | | SVPDPQPLGQ PPASPSGLGT NTMATGSGAP MADNNEGADG VGNSSGNWHC DSTWMGDRVV |
| | | TTSTRTWALP TYNNHLYKQI SSQSGASNDN HYFGYSTPWG YFDFNRFHCH FSPRDWQRLI |
| | | NNNWGFRPKR LNFKLFNIQV KEVTQNDGTT TIANNLTSTV QVFTDLEYQL PYVLGSAHQG |
| | | CLPPFPADVF MVPCYGYLTL NNGSCAVGRS SFYCLEYFPS QMLRTGNNFT FSYTFEDVPF |
| | | HSSYAHSQSL DRLMNPLIDQ YLYYLSRTNT PSGTTTQSRL QFSQAGASDI RDCSRNWLPG |
| | | PCYRQQRVSK TAADNNNSEY SWTGATKYHL NGRDSLVNPG PAMASHKDDE EKFFPQSGVL |
| | | IFGKQGSEKT NVDIEKVMIT DEEEIRTTNP VATEQYGSVS INLQSGNTQA ATSDVNTQGV |
| | | LPGMVWQDRD VYLCGPIWAK IPHTDGHFHP SPLMGGFGLK HPPPQILIKN TPVPANPSTT |
| | | FSAAKFASFI TQYSTGQVSV EIEWELQKEN SKRWNPEIQY TSNYNKSVNV DFTVDINGVY |
| | | SEPRPIGTRY LTRNL (SEQ ID NO: 86) |
| AAV.rh. | — | MAADGYLPDW LEDNLSEGIR EWWDIKPGAP KPKANQQKQD DGRGLVLPGY KYLGPFNGLD |
| 24 | | KGEPVNAADA AALEHDKAYD QQLKAGDNPY LRYNHADAEF QERLQEDTSF GGNLGRAVFQ |
| | | AKKRVLEPLG LVEEGAKTAP GKKRPIESPD SSTGIGKKGQ QPAKKKLNFG QTGDSESVPD |
| | | PQPIGEPPAG PSGIGSGTMA AGGGAPMADN NEGADGVGSS SGNWHCDSTW LGDRVITTST |
| | | RTWALPTYNN HLYKQISNGT SGGSTNDNTY FGYSTPWGYF DFNRFHCHFS PREWQRLINN |
| | | NWGFRPKRLN FKLFNIQVKE VTQNEGTKTI ANNLTSTIQV FTDSEYQLPY VLGSAHQGCL |
| | | PPFPADVFMI PQYGYLTLNN GSQAVGRSSF YCLEYFPSQM LRTGNNFEFS YQFEDVPFHS |
| | | SYAHSQSLDR LMNPLIDQYL YYLSRTQSTG GTAGTQQLLF SQAGPNNMSA QAKNWLPGPC |
| | | YRQQRVSTTV SQNNNSNFAW TGATKYHLNG RDSLVNPGVA MATHKGDEER FFPSSGVLMF |
| | | GKQGAGKDNV DYSSVMLTSE EEIKTTNPVA TEQYGVVADN LQQQNAAPIV GAVNSQGALP |
| | | GMVWQNRDVY LQGPIWAKIP HTDGNFHPSP LMGGFGLKHP PPQILIKNTP VPADPPTTFS |
| | | QAKLASFITQ YSTGQVSVEI EWELCKENSK RWNPEIQYTS NYYKSTNVDF AVNTEGTYSE |
| | | PRPIGTRYLT RSL (SEQ ID NO: 87) |
| AAV.hu. | — | MAADGYLPDW LEDNLSEGIR EWWDIKPGAP KPKANQQKQD DGRGLVLPGY KYLGPFNGLD |
| 38 | | KGEPVNAADA AALEHDKAYD QQLKAGDNPY LRYNHADAEF QERLQEDTSF GGNLGRAVFQ |
| | | AKKRVLEPLG LVEEAAKTAP GKKRPVEPSP QRSPDSSTGI GKKGQQPAKK RLNFGQTGDS |
| | | ESVPDPQPIG EPPAGPSGLG SGTMAAGGGA PMADNNEGAD GVGSSSGNWH CDSTWLGDRV |
| | | ITTSTRTWAL PTYNNHLYKQ ISNGTSGGST NDNTYFGYST PWGYFDFNRF HCHFSPRDWQ |
| | | RLINNNWGFR PKRLSFKLEN IQVKEVTQNE GTKTIANNLT STIQVFTDSE YQLPYVLGSA |
| | | HQGCLPPFPA DVFMIPQYGY LTLNNGSQAV GRSSFYCLEY FPSQMLRTGN NFEFSYTFED |
| | | VPFHSSYAHS QSLDRLMNPL IDQYLYYLSR TQSTGGTQGT QQLLFSQAGP ANMSAQAKNW |
| | | LPGPCYRQQR VSTTLSQNNN SNFAWTGATK YHLNGRDSLV NPGVAMATHK DDEERFFPSS |
| | | GVLMFGKQGA GRDNVDYSSV MLTSEEEIKT TNPVATEQYG VVADNLQQTN TGPIVGNVNS |
| | | QGALPGMVWQ NRDVYLQGPI WAKIPHTDGN CHPSPLMGGF GLKHPPPQIL IKNTPVPADP |
| | | PTTFSQAKLA SFITQYSTGQ VSVEIEWELQ KENSKRWNPE IQYTSNYYKS TNVDFAVNTE |
| | | GTYSEPRPIG TRYITRNL (SEQ ID NO: 88) |
| AAV.rh. | — | MAADGYLPDW LEDNLSEGIR EWWDIKPGAP KPKANQQKQD DGRGLVLPGY KYLGPFNGLD |
| 72 | | KGEPVNAADA AALEHDKAYD QQLKAGDNPY LRYNHADAEF QERLQEDTSF GGNLGRAVFQ |
| | | AKKRVLEPLG LVEEAAKTAP GKKRPVEPSP QRSPDSSTGI GKKGQQPAKK RLNFGQTGDS |
| | | ESVPDPQPIG EPPAGPSGLG SGTMAAGGGA PMADNNEGAD GVGSSSGNWH CDSTWLGDRV |
| | | ITTSTRTWAL PTYNNHLYKQ ISNGTSGGST NDNTYFGYST PWGYFDFNRF HCHFSPRDWQ |
| | | RLINNNWGFR PKRLSFKLEN IQVKEVTQNE GTKTIANNLT STIQVFTDSE YQLPYVLGSA |
| | | HQGCLPPFPA DVFMIPQYGY LTLNNGSQAV GRSSFYCLEY FPSQMLRTGN NFEFSYTFED |
| | | VPFHSSYAHS QSLDRLMNPL IDQYLYYLSR TQSTGGTQGT QQLLFSQAGP ANMSAQAKNW |
| | | LPGPCYRQQR VSTTLSQNNN SNFAWTGATK YHLNGRDSLV NPGVAMATHK DDEERFFPSS |
| | | GVLMFGKQGA GRDNVDYSSV MLTSEEEIKT TNPVATEQYG VVADNLQQTN TGPIVGNVNS |
| | | QGALPGMVWQ NRDVYLQGPI WAKIPHTDGN FHPSPLMGGF GLKHPPPQIL IKNTPVPADP |
| | | PTTFSQAKLA SFITQYSTGQ VSVEIEWELQ KENSKRWNPE IQYTSNYYKS TNVDFAVNTE |
| | | GTYSEPRPIG TRYLTRNL (SEQ ID NO: 89) |
| AAV.cy. | — | MAADGYLPDW LEGNLSEGIR EWWDIKPGAP KPKANQQKQD DGRGLVLPGY RYLGPENGLD |
| 5 | | KGEPVNEADA AALEHDKAYD KQLECGDNPY LKYNHADAEF QERLQEDTSF GGNLGRAVFQ |
| | | AKKRVLEPLG LVEEGAKTAP GKKRPIESPD SSTGIGKNGQ PPAKKKINFG QTGDSESVPD |
| | | PQPLGEPPAA PSGLGSGTMA AGGGAPMADN NEGADGVGNA SGNWHCDSTW LGDRVITTST |
| | | RTWALPTYNN HLYKQISSQS GATNENHFFG YSTPWGYFDF NRFHCHFSPR DWQRLINNNW |
| | | GFRPRKLRFK LFNIQVKEVT TNDGVTTIAN NLTSTIQVFS DSEYQLPYVL GSAHQGCLPP |
| | | FPADVFMIPQ YGYITINNGS QSVGRSSFYC LEYFPSQMLR TGDNFEFSYT FEEVPFHSSY |
| | | AHSQSLDRLM NPLIDQYLYY LARTQSTTGS TRELQFHQAG PNTMAEQSKN WLPGPCYRQQ |
| | | RLSKNIDSNN NSNFAWTGAT KYHLNGRNSL TNPGVAMATN KDDEDQFFPI NGVLVFGKTG |
| | | AANKTTLENV LMTSEEEIKT TNPVATEEYG VVSSNLQSST AGPQTQTVNS QGALPGMVWQ |
| | | NRDVYLQGPI WAKIPHTDGN FHPSPLMGGF GLKHPPPQIL IKNTPVPANP PEVFTPAKFA |
| | | SFITQYSTGQ VSVEIEWELQ KENSKRWNPE IQYTSNYAKS NNVEFAVNNE GVYTEPRPIG |
| | | TRYLTRNL (SEQ ID NO: 90) |
| AAV.cy. | — | MAADGYLPDW LEDNLSEGIR EWWDLKPGAP KPKANQQKQD DGRGLVLPGY KYLGPENGLD |
| 6 | | KGEPVNEADA AALEHDKAYD KQLECGDNPY LKYNHADAEF QERLQEDTSF GGNLGRAVFQ |
| | | AKKRVLEPLG LVEEGAKTAP GKKRPIESPD SSTGIGKKGQ QPAKKKLNFG QTGDSESVPD |
| | | PQPLGEPPAA PSGIGSGTMA AGGGAPMADN NEGADGVGNA SGNWHCDSTW LGERVITTST |
| | | RTWALPTYNN HLYKQISSQS GATNENHFFG YSTPWGYFDF NRFHCHFSPR DWQRLINNNW |
| | | GFRPRKLRFK LFNIQVKEVT TNDGVTTIAN NLTSTIQVFS DSEYQLPYVL GSAHQGCLPP |
| | | FPADVFMIPQ YGYLTLNNGS QSMGRSSFYC LEYFPSQMLR TGNNFEFSYT FEEVPFHSSY |
| | | AHSQSLDRLM NPLIDQYLYY LARTQSTTGS TRELQFHQAG PNTMAEQSKN WLPGPCYRQQ |
| | | RLSKNIDSNN NSNFAWTGAT KYHLNGRNSL TNPGVAMATN KDDEGQFFPI NGVLVFGKTG |
| | | AANKTTLENV LMTSEEEIKT TNPVATEEYG VVSSNLQSST AGPQTQTVNS QGALPGMVWQ |
| | | NRDVYLQGPI WAKIPHTDGN FHPSPLMGGF GLKHPPPQIL IKNTPVPANP PGVFTPALFA |
| | | SFITQYSTGQ VSVEIEWELQ KENSKRWNPE IQYTSNYAKS NNVEFAVNNE GVYTEPRPIG |
| | | TRYLTRNL (SEQ ID NO: 91) |
| AAV.rh. | — | MAADGYLPDW LEDNLSEGIR EWWDIKPGAP KPKANQQKQD DGRGLVLPGY KYLGPENGLD |
| 46 | | KGEPVNAADA AALEHDKAYD QQLKAGDNPY LRYNHADAEF QERLQEDTSF GGNLGRAVFQ |
| | | AKKRVLEPLG LVEEGAKTAP GKKRPVEPSP QRSPDSSTGI GKKGQQPARK RLNFGQTGDS |
| | | ESVPDPQPIG EPPAAPSSVG SGTMAAGGGA PMADNNEGAD GVGSSSGNWH CDSTWLGDRV |
| | | ITTSTRTWAL PTYNNHLYKQ ISNGTSGGST NDNTYFGYST PWGYFDFNRF HCHFSPRDWQ |
| | | RLINNNWGFR PKRLSFKLFN IQVKEVTQNE GTKTIANNLT STIQVFTDSE YQLPYVIGSA |
| | | HQGCLPPFPA DVFMIPQYGY LTINNGSQAV GRSSFYCLEY FPSQMLRTGN NFSFSYTFED |
| | | VPFHSSYAHS QSLDRLMNPL IDQYLYYLSR TQSTGGTAGT QQLLFSQAGP SNMSAQARNW |
| | | LPGPCYRQQR VSTTLSQNNN SNFAWTGATK YHLNGRDSLV NPGVAMATNK DDEDRFFPSS |
| | | GILMFGKQGA GKDNVDYSNV MLTSEEEIKA TNPVATEQYG VVADNLQQQN TAPIVGAVNS |
| | | QGALPGMVWQ NRDVYLQGPI WAKIPHTDGN FHPSPLMGGF GLKHPPPQIL IKNTPVPADP |
| | | PTAFNQAKLN SFITQYSTGQ VSVEIEWELQ KENSKRWNPE IQYTSNYYKS TNVDFAVNTE |
| | | GVYSEPRPIG TRYLTRNL (SEQ ID NO: 92) |
| AAV.rh. | — | MAADGYLPDW LEDNLSEGIR EWWDIKPGAP KPKANQQKQD DGRGLVLPGY KYLGPENGLD |
| 2 | | KGEPVNAADA AALEHDKAYD QQLKAGDNPY LRYNHADAEF QERLQEDTSF GGNLGRAVFQ |
| | | AKKRVLEPLG LVEEGAKTAP GKKRPVEPSP QRSPDSSTGI GKKGHQPARK RLNFGQTGDS |
| | | ESVPDPQPIG EPPAGPSGLG SGTMAAGGGA PMADNNEGAD GVGSSSGNWH CDSTWLGDRV |
| | | ITTSTRTWAL PTYNNHLYKQ ISNGTSGGST NDNTYFGYST PWGYFDFNRF HCHFSPRDWQ |
| | | RLINNNWGFR PKRINFKLEN IQVKEVTQNE GTKTIANNLT STIQVFTDSE YQLPYVPGSA |
| | | HQGCLPPFPA DVFMIPQYGY LTLNNGSQAV GRSSFYCLEY FPSQMLRTGN NFEFSYTFED |
| | | VPFHSSYAHS QSLDRLMNPL IDQYLYYLSR TQSTGGTQGT QQLLFSQAGP ANMSAQAKNW |
| | | LPGPCYRQQR VSTTLSQNNN SNFAWTGATK YHLNGRDSLV NPGVAMATHK DDEERFFPSS |
| | | GVLMFGKQGA GKDNVDYSSV MLTSEEEIKT TNPVATEQYG VVADNLQQTN GAPIVGTVNS |
| | | QGALPGMVWQ NRDVYLQGPI WAKIPHTDGN FHPSPLMGGF GLKHPPPQIL VKNTPVPADP |
| | | PTTFSQAKLA SFITQYSTGQ VSVEIEWELQ KENSKRWNPE IQYTSNYYKS TNVDFAVNTE |
| | | GTYSEPRPIG TRYITRNL (SEQ ID NO: 93) |
| Rh.64R1 | — | MAADGYLPDW LEDNLSEGIR EWWDLKPGAP KPKANQQKQD DGRGLVLPGY |
| | | KYLGPFNGLD KGEPVNAADA AALEHDKAYD QQLKAGDNPY LRYNHADAEF |
| | | QERLQEDTSF GGNLGRAVFQ AKKRVLEPLG LVEEGAKTAP GKKRPVEPSP |
| | | QRSPDSSTGI GKKGQQPARK RLNFGQTGDS ESVPDPQPIG EPPAAPSSVG |
| | | SGTMAAGGGA PMADNNEGAD GVGSSSGNWH CDSTWLGDRV ITTSTRTWAL |
| | | PTYNNHLYKQ ISNGTSGGST NDNTYFGYST PWGYFDFNRF HCHFSPRDWQ |
| | | RLINNNWGFR PKRLSFKLEN IQVKEVTQNE GTKTIANNLT STIQVFTDSE |
| | | YQLPYVLGSA HQGCLPPFPA DVFMIPQYGY LTLNNGSQAV GRSSFYCLEY |
| | | FPSQMLRTGN NFSFSYTFED VPFHSSYAHS QSLDRIMNPL IDQYLYYLSR |
| | | TQSTGGTAGT QQLLFSQAGP SNMSAQARNW LPGPCYRQQR VSTTLSQNNN |
| | | SNFAWTGATK YHLNGRDSLV NPGVAMATNK DDEDRFFPSS GILMFGKQGA |
| | | GKDNVDYSNV MLTSEEEIKT TNPVATEQYG VVADNLQQQN TAPIVGAVNS |
| | | QGALPGMVWQ NRDVYLQGPI WAKIPHTDGN FHPSPLMGGF GLKHPPPQIL |
| | | IKNTPVPADP PTAFNQAKLN SFITCYSTGQ VSVEIVWELQ KENSKRWNPE |
| | | IQYTSNYYKS VTVDFAVNTE GVYSEPRPIG TRYLTRNL (SEQ ID NO: 107) |

### 7. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

We claim:

1. A method of delivering a transgene to an ocular tissue cell, said method comprising contacting said cell with an rAAV vector comprising a transgene encoding an ocular disease therapeutic operably linked to one or more regulatory elements that promote expression of the ocular disease therapeutic in the ocular tissue cell, wherein the rAAV has a capsid of AAV1 (SEQ ID NO: 59); AAV2 (SEQ ID NO:60); AAV3 SEQ ID NO:61); AAV3B (SEQ ID NO:74); AAV4 (SEQ ID NO:62); AAV5 (SEQ ID NO:63); AAV6 (SEQ ID NO:64); AAV7 (SEQ ID NO:65); AAV8 (SEQ ID NO:66); AAV9 (SEQ ID NO:67); AAV9e (SEQ ID NO:68); AAVrh.10 (SEQ ID NO:69); AAVrh.20 (SEQ ID NO:70); AAVhu.37 (SEQ ID NO:71); AAVrh39 (SEQ ID NO:73); AAV rh73 (SEQ ID NO:75); AAVrh.74 (SEQ ID NO:72 or SEQ ID NO:96); AAVhu.51 (SEQ ID NO:76); AAVhu.21 (SEQ ID NO:77); AAVhu.12 (SEQ ID NO:78); AAVhu.26 (SEQ ID NO:79); AAVrh.24 (SEQ ID NO:87); AAVhu.38 (SEQ ID NO:88); AAVrh.72 (SEQ ID NO:89); AAVhu.56 (SEQ ID NO:86); AAVcy.5 (SEQ ID NO:90); AAVcy.6 (SEQ ID NO:91); AAVrh.46 (SEQ ID NO:92); AAVrh.13 (SEQ ID NO:85); AAVrh.64.R1 (SEQ ID NO:107); AAV9.S454-TFR3 (SEQ ID NO: 42); AAV8.BBB (SEQ ID NO: 26); AAV8.BBB.LD (SEQ ID NO:27); AAV8.Y703F (Y703F substitution in the amino acid sequence of SEQ ID NO:66, see FIG. 7 for numbering); AAV9.Y443F (Y443F substitution in the amino acid sequence of SEQ ID NO:67, see FIG. 7 for numbering); or AAV9.Y6F (Y6F substitution in the amino acid sequence of SEQ ID NO:66, see FIG. 7 for numbering).

2. A method of delivering a transgene to ocular tissue, or an ocular tissue target cell or cellular matrix thereof, of a subject in need thereof, said method comprising administering to said subject an rAAV vector comprising a transgene encoding an ocular disease therapeutic operably linked to one or more regulatory elements that promote expression of the ocular disease therapeutic in the ocular tissue, wherein the rAAV has a capsid AAV1 (SEQ ID NO: 59); AAV2 (SEQ ID NO:60); AAV3 SEQ ID NO:61); AAV3B (SEQ ID NO:74); AAV4 (SEQ ID NO:62); AAV5 (SEQ ID NO:63); AAV6 (SEQ ID NO:64); AAV7 (SEQ ID NO:65); AAV8 (SEQ ID NO:66); AAV9 (SEQ ID NO:67); AAV9e (SEQ ID NO:68); AAVrh.10 (SEQ ID NO:69); AAVrh.20 (SEQ ID NO:70); AAVhu.37 (SEQ ID NO:71); AAVrh39 (SEQ ID NO:73); AAV rh73 (SEQ ID NO:75); AAVrh.74 (SEQ ID NO:72 or SEQ ID NO:96); AAVhu.51 (SEQ ID NO:76); AAVhu.21 (SEQ ID NO:77); AAVhu.12 (SEQ ID NO:78); AAVhu.26 (SEQ ID NO:79); AAVrh.24 (SEQ ID NO:87); AAVhu.38 (SEQ ID NO:88); AAVrh.72 (SEQ ID NO:89); AAVhu.56 (SEQ ID NO:86); AAVcy.5 (SEQ ID NO:90); AAVcy.6 (SEQ ID NO:91); AAVrh.46 (SEQ ID NO:92); AAVrh.13 (SEQ ID NO:85); AAVrh.64.R1 (SEQ ID NO:107); AAV9.S454-TFR3 (SEQ ID NO: 42); AAV8.BBB (SEQ ID NO: 26); AAV8.BBB.LD (SEQ ID NO:27); AAV8.Y703F (Y703F substitution in the amino acid sequence of SEQ ID NO:66, see FIG. 7 for numbering); AAV9.Y443F (Y443F substitution in the amino acid sequence of SEQ ID NO:67, see FIG. 7 for numbering); or AAV9.Y6F (Y6F substitution in the amino acid sequence of SEQ ID NO:66, see FIG. 7 for numbering).

3. The method of claim 1 or 2, wherein the capsid is an AAV3B serotype, AAVrh.73 serotype, AAV.hu.26 serotype, AAVhu.51, AAVrh64R1 serotype or AAV9.S454.TFR3 capsid.

4. The method of any of claims 1 to 3, in which the ocular tissue or ocular tissue target cell is a cornea tissue or cell, iris tissue or cell, ciliary body tissue or cell, Schlemm's canal tissue or cell, trabecular meshwork tissue or cell, retinal tissue or cell, RPE-choroid tissue or cell, or optic nerve cell.

5. The method of claim 4, wherein the ocular tissue or ocular tissue target cell is a retinal tissue or cell or an RPE-choroid tissue or cell.

6. The method of claim 5, wherein the capsid is an AAV3B or AAVrh.73 capsid.

7. The method of claims 1 to 6, wherein the ocular disease is non-infectious uveitis.

8. The method of claims 1 to 4, wherein the ocular disease is glaucoma.

9. The method of claim 8 wherein said rAAV targets the trabecular meshwork and/or the Schlemm's canal.

10. The method of claim 8 or 9 wherein the capsid is an AAV1 capsid, AAV2, AAV7 capsid, AAV3B capsid, AAV.hu.26 capsid, or AAV9.S454-TFR3 capsid.

11. The method of any of claims 1 to 10, wherein said rAAV vector is administered intravitreally, suprachoroidally, or intracamerally.

12. The method of any of claims 1 to 10 wherein said rAAV vector is administered systemically.

13. The method of any of claims 1 to 12, wherein provided said rAAV vector is administered in the absence of hyaluronic acid.

14. A pharmaceutical composition for use in delivering a transgene to an ocular tissue cell, said composition comprising an rAAV vector comprising a transgene encoding an ocular disease therapeutic operably linked to one or more regulatory elements that promote expression of the ocular disease therapeutic in the ocular tissue cell, wherein the rAAV has a capsid of AAV1 (SEQ ID NO: 59); AAV2 (SEQ ID NO:60); AAV3 SEQ ID NO:61); AAV3B (SEQ ID NO:74); AAV4 (SEQ ID NO:62); AAV5 (SEQ ID NO:63); AAV6 (SEQ ID NO:64); AAV7 (SEQ ID NO:65); AAV8 (SEQ ID NO:66); AAV9 (SEQ ID NO:67); AAV9e (SEQ ID NO:68); AAVrh.10 (SEQ ID NO:69); AAVrh.20 (SEQ ID NO:70); AAVhu.37 (SEQ ID NO:71); AAVrh39 (SEQ ID NO:73); AAV rh73 (SEQ ID NO:75); AAVrh.74 (SEQ ID NO:72 or SEQ ID NO:96); AAVhu.51 (SEQ ID NO:76); AAVhu.21 (SEQ ID NO:77); AAVhu.12 (SEQ ID NO:78); AAVhu.26 (SEQ ID NO:79); AAVrh.24 (SEQ ID NO:87); AAVhu.38 (SEQ ID NO:88); AAVrh.72 (SEQ ID NO:89); AAVhu.56 (SEQ ID NO:86); AAVcy.5 (SEQ ID NO:90); AAVcy.6 (SEQ ID NO:91); AAVrh.46 (SEQ ID NO:92); AAVrh.13 (SEQ ID NO:85); AAVrh.64.R1 (SEQ ID NO:107); AAV9.S454-TFR3 (SEQ ID NO: 42); AAV8.BBB (SEQ ID NO: 26); AAV8.BBB.LD (SEQ ID NO:27); AAV8.Y703F (Y703F substitution in the amino acid sequence of SEQ ID NO:66, see FIG. 7 for numbering); AAV9.Y443F (Y443F substitution in the amino acid sequence of SEQ ID NO:67, see FIG. 7 for numbering); or AAV9.Y6F (Y6F substitution in the amino acid sequence of SEQ ID NO:66, see FIG. 7 for numbering).

15. The pharmaceutical composition of claim 14, wherein the capsid is an AAV3B serotype, AAVrh.73 serotype, AAV.hu.26 serotype, AAVhu.51, AAVrh64R1 serotype or AAV9.S454.TFR3 capsid.

16. The pharmaceutical composition of claim 14 or 15, in which the ocular tissue or ocular tissue target cell is a cornea tissue or cell, iris tissue or cell, ciliary body tissue or cell, Schlemm's canal tissue or cell, trabecular meshwork tissue or cell, retinal tissue or cell, RPE-choroid tissue or cell, or optic nerve cell.

17. The pharmaceutical composition of claim 16, wherein the ocular tissue or ocular tissue target cell is a retinal tissue or cell or an RPE-choroid tissue or cell.

18. The pharmaceutical composition of claim 17, wherein the capsid is an AAV3B or AAVrh.73 capsid.

19. The pharmaceutical composition of claims 14 to 18, wherein the ocular disease is non-infectious uveitis.

20. The pharmaceutical composition of claims 14 to 18, wherein the ocular disease is glaucoma.

21. The pharmaceutical composition of claim 20 wherein said rAAV targets the trabecular meshwork and/or the Schlemm's canal.

22. The pharmaceutical composition of claim 20 or 21 wherein the capsid is an AAV3B serotype, AAVrh.73 serotype, AAV.hu.26 serotype, AAVhu.51, AAVrh64R1 serotype or AAV9.S454.TFR3 capsid.

23. The pharmaceutical composition of any of claims 14 to 22, wherein said rAAV vector is administered intravitreally, suprachoroidally, or intracamerally.

24. The method of any of claims 14 to 22 wherein said rAAV vector is administered systemically.

25. The method of claims 14 to 24, wherein provided said rAAV vector is administered in the absence of hyaluronic acid.

26. The method or pharmaceutical composition of any of claims 1 to 25 wherein the rAAV exhibits at least 1.1-fold, 1.5-fold, 2-fold, 3-fold, 4-fold, 5-fold, 6-fold, 7-fold, 8-fold, 9-fold, or 10-fold greater transduction in the target tissue, compared to a reference AAV capsid.

27. The method or pharmaceutical composition of any of claims 1 to 26 wherein the abundance of transgene RNA is 1.1-fold, 1.5-fold, 2-fold, 3-fold, 4-fold, 5-fold, 6-fold, 7-fold, 8-fold, 9-fold, or 10-fold greater in the target tissue compared to the abundance of transgene RNA from the reference AAV capsid.

28. The method or pharmaceutical composition of claim 26 or 27 where the reference AAV capsid is AAV2, AAV8 or AAV9

29. A method of treating an ocular disorder in a subject in need thereof, said method comprising administering a therapeutically effective amount of the pharmaceutical composition of any of claim 14-22 or 25.

30. A nucleic acid comprising a nucleotide sequence encoding the rAAV capsid protein of any of the above claims, or encoding an amino acid sequence sharing at least 80% identity therewith.

31. A packaging cell capable of expressing the nucleic acid of claim 30 to produce AAV vectors comprising the capsid protein encoded by said nucleotide sequence.


SEQUENCE LISTING (107 sequences)

SEQ ID NO: 1 - protein, 7 aa, Artificial Sequence
  synthetic construct; capsid peptide
TLAAPFK

SEQ ID NO: 2 - protein, 8 aa, Artificial Sequence
  synthetic construct; capsid peptide
DDDDDDDD

SEQ ID NO: 3 - protein, 7 aa, Artificial Sequence
  synthetic construct; capsid peptide
LSSRLDA

SEQ ID NO: 4 - protein, 9 aa, Artificial Sequence
  synthetic construct; capside peptide
CLSSRLDAC

SEQ ID NO: 5 - protein, 7 aa, Artificial Sequence
  synthetic construct; capside peptide
CLPVASC

SEQ ID NO: 6 - protein, 5 aa, Artificial Sequence
  synthetic construct; capside peptide
LPVAS

SEQ ID NO: 7 - protein, 7 aa, Artificial Sequence
  synthetic construct; capside peptide
ASSLNIA

SEQ ID NO: 8 - protein, 7 aa, Artificial Sequence
  synthetic construct; capside peptide
LGETTRP

SEQ ID NO: 9 - protein, 10 aa, Artificial Sequence
  synthetic construct; capside peptide
LALGETTRPA

SEQ ID NO: 10 - protein, 7 aa, Artificial Sequence
  synthetic construct; capside peptide
HAIYPRH

SEQ ID NO: 11 - protein, 12 aa, Artificial Sequence
  synthetic construct; capsid peptide
THRPPMWSPVWP

SEQ ID NO: 12 - protein, 7 aa, Artificial Sequence
  synthetic construct; capsid peptide
RTIGPSV

SEQ ID NO: 13 - protein, 9 aa, Artificial Sequence
  synthetic construct; capsid peptide
CRTIGPSVC

SEQ ID NO: 14 - protein, 11 aa, Artificial Sequence
  synthetic construct; capsid peptide
SITLVKSTQTV

SEQ ID NO: 15 - protein, 10 aa, Artificial Sequence
  synthetic construct; capsid peptide
TILSRSTQTG

SEQ ID NO: 16 - protein, 7 aa, Artificial Sequence
  synthetic construct; capsid peptide
QAVRTSL

SEQ ID NO: 17 - protein, 7 aa, Artificial Sequence
  synthetic construct; capsid peptide
QAVRTSH

SEQ ID NO: 18 - protein, 20 aa, Artificial Sequence
  synthetic construct; capsid peptide
VVMVGEKPITITQHSVETEG

SEQ ID NO: 19 - protein, 12 aa, Artificial Sequence
  synthetic construct; capsid peptide
RSSEEDKSTQTT

SEQ ID NO: 20 - protein, 7 aa, Artificial Sequence
  synthetic construct; capsid peptide
TLAVPFK

SEQ ID NO: 21 - protein, 8 aa, Artificial Sequence
  synthetic construct; capsid peptide
DYKDDDDK

SEQ ID NO: 22 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSHAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 27 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQAAASNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 28 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNGTSGGSTNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQAAASNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 31 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 32 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQAAASEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFARPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 34 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVAGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 744 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSDDDDDDDDGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 36 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLSSRLDAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - protein, 745 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLSSRLDACGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 38 - protein, 741 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSLPVASGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCLPVASCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSASSLNIAGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 41 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSHAIYPRHGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 743 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSRTIGPSVGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 745 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSCRTIGPSVCGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 44 - protein, 747 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSITLVKSTQTVSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 746 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNTILSRSTQTGSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 753 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTTILSRSTQTGAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 754 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTSITLVKSTQTVAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 35 aa, Artificial Sequence
  synthetic construct; capsid sequence
  misc_feature (14)..(15): Xaa can be any naturally occurring amino acid
  misc_feature (23)..(23): Xaa can be any naturally occurring amino acid
  misc_feature (30)..(30): Xaa can be any naturally occurring amino acid
YYLSRTQNTGGTAXXQTLLFSQXGPSNMSXQAKNW

SEQ ID NO: 49 - protein, 35 aa, Artificial Sequence
  synthetic construct; capsid sequence
YYLNRTQNQSGSAQNKDLLFSRGSPAGMSVQPKNW

SEQ ID NO: 50 - protein, 35 aa, Artificial Sequence; identical to SEQ ID NO: 49
  synthetic construct; capsid sequence

SEQ ID NO: 51 - protein, 35 aa, Artificial Sequence
  synthetic construct; capsid sequence
YYLSRTNTPSGTTTQSRLQFSQAGASDIRDQSRNW

SEQ ID NO: 52 - protein, 36 aa, Artificial Sequence
  synthetic construct; capsid sequence
YYLNRTQGTTSGTTNQSRLLFSQAGPQSMSLQARNW

SEQ ID NO: 53 - protein, 36 aa, Artificial Sequence
  synthetic construct; capsid sequence
WGLQSTTTGTTLNAGTATTNFTKLRPTNFSNFKKNW

SEQ ID NO: 54 - protein, 29 aa, Artificial Sequence
  synthetic construct; capsid sequence
YRFVSTNNTGGVQFNKNLAGRYANTYKNW

SEQ ID NO: 55 - protein, 36 aa, Artificial Sequence
  synthetic construct; capsid sequence
YYLARTQSNPGGTAGNRELQFYQGGPSTMAEQAKNW

SEQ ID NO: 56 - protein, 35 aa, Artificial Sequence
  synthetic construct; capsid sequence
YYLSRTQTTGGTANTQTLGFSQGGPNTMANQAKNW

SEQ ID NO: 57 - protein, 34 aa, Artificial Sequence
  synthetic construct; capsid sequence
YYLSKTINGSGQNQQTLKFSVAGPSNMAVQGRNY

SEQ ID NO: 58 - protein, 35 aa, Artificial Sequence
  synthetic construct; capsid sequence
YYLSRTQSTGGTAGTQQLLFSQAGPNNMSAQAKNW

SEQ ID NO: 59 - protein, 736 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 60 - protein, 735 aa, Artificial Sequence [sequence omitted]

SEQ ID NO: 61 - protein, 736 aa, Artificial Sequence
  synthetic construct; capsid sequence
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 62 - protein, 734 aa, Artificial Sequence
  synthetic construct; capsid sequence
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 63 - protein, 724 aa, Artificial Sequence
  synthetic construct; capsid sequence
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 64 - protein, 736 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 65 - protein, 737 aa, Artificial Sequence
  synthetic construct; capsid sequence
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 66 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 67 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 68 - protein, 736 aa, Artificial Sequence
  synthetic construct; capsid sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 70 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 71 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 72 - protein, 738 aa, Artificial Sequence
  synthetic construct; capsid sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 73 - protein, 738 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 74 - protein, 736 aa, Artificial Sequence
  synthetic construct; capsid sequence
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 75 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 76 - protein, 736 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSGYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 77 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 78 - protein, 735 aa, Artificial Sequence
  synthetic construct; capsid sequence
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 79 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 80 - protein, 734 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 81 - protein, 733 aa, Artificial Sequence
  synthetic construct; capsid sequence
MAADGYLPDWLEDNLSEGIREWWDLKPGAPKPKANQQKQDDGRGLVLPGYEYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFETAYNFEKVPFHSMYAHSQSLDGLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPAYPATTFTAARVDSFITQYSTGQVAVQIEWEIEKERSKRWNPEVQFTSNCGNQSSMLWAPDTTGKYTEPRVIGSRYLTNHL

SEQ ID NO: 82 - protein, 736 aa, Artificial Sequence
  synthetic construct; capsid sequence
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 83 - protein, 644 aa, Artificial Sequence
  synthetic construct; capsid sequence
  misc_feature (434)..(434): Xaa can be any naturally occurring amino acid
KAYDQQLKAGDNPYLRYNHADAEFQERLQEDTSFGGNLGRAVFQAKKRVLEPLGLVETPAKTAPGKKRPVDSPDSTSGIGKKGQQPAKKRLNFGQTGDSESVPDPQPIGEPPAGPSGLGSGTMAAGGGAPMADNNEGADGVGNASGNWHCDSTWLGDRVITTSTRTWALPTYNNHLYKQISSQSAGSTNDNVYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNAGGTAGNRELQFYQGGPTTMAEQAKNWLPGPCFRQQRVSKTLDQNNNSNFAWTGATKYHLNXRNSLVNPGVAMATHKDDEERFFPSSGVLIFGKTGAANKTTLENVLMTNEEEIRPTNPVATEEYGIVSSNLQAASTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKQTGVDFAVDSQGVYSEP

SEQ ID NO: 84 - protein, 735 aa, Artificial Sequence; identical to SEQ ID NO: 78
  synthetic construct; capsid sequence

SEQ ID NO: 85 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQGGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 86 - protein, 735 aa, Artificial Sequence
  synthetic construct; capsid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGNQPARKRLNFGQTGDADSVPDPQPLGQPPASPSGLGTNTMATGSGAPMADNNEGADGVGNSSGNWHCDSTWMGDRVVTTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDLEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTAADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 87 - protein, 733 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPIESPDSSTGIGKKGQQPAKKKLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTVSQNNNSNFAWTGATKYHLNGRDSLVNPGVAMATHKGDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRSL

SEQ ID NO: 88 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNCHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 89 - protein, 738 aa, Artificial Sequence; identical to SEQ ID NO: 71
  synthetic construct; capsid sequence

SEQ ID NO: 90 - protein, 728 aa, Artificial Sequence
  synthetic construct; capsid sequence
MAADGYLPDWLEGNLSEGIREWWDLKPGAPKPKANQQKQDDGRGLVLPGYRYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPIESPDSSTGIGKNGQPPAKKKLNFGQTGDSESVPDPQPLGEPPAAPSGLGSGTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGDNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEDQFFPINGVLVFGKTGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNYAKSNNVEFAVNNEGVYTEPRPIGTRYLTRNL

SEQ ID NO: 91 - protein, 728 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNEADAAALEHDKAYDKQLEQGDNPYLKYNHADAEFQERLQEDTSFGGNLGRAVFQAKKRVLEPLGLVEEGAKTAPGKKRPIESPDSSTGIGKKGQQPAKKKLNFGQTGDSESVPDPQPLGEPPAAPSGLGSGTMAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPRKLRFKLFNIQVKEVTTNDGVTTIANNLTSTIQVFSDSEYQLPYVLGSAHQGCLPPFPADVFMIPQYGYLTLNNGSQSMGRSSFYCLEYFPSQMLRTGNNFEFSYTFEEVPFHSSYAHSQSLDRLMNPLIDQYLYYLARTQSTTGSTRELQFHQAGPNTMAEQSKNWLPGPCYRQQRLSKNIDSNNNSNFAWTGATKYHLNGRNSLTNPGVAMATNKDDEGQFFPINGVLVFGKTGAANKTTLENVLMTSEEEIKTTNPVATEEYGVVSSNLQSSTAGPQTQTVNSQGALPGMVWQNRDVYLQGPIWAKIPHTDGNFHPSPLMGGFGLKHPPPQILIKNTPVPANPPGVFTPALFASFITQYSTGQVSVEIEWELQKENSKRWNPEIQYTSNYAKSNNVEFAVNNEGVYTEPRPIGTRYLTRNL

SEQ ID NO: 92 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 93 - protein, 738 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGHQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVPGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQTNGAPIVGTVNSQGALPGMVWQNRDVYLQGPIWAKIPHTDGNFHPSPLMGGFGLKHPPPQILVKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 94 - protein, 736 aa, Artificial Sequence; identical to SEQ ID NO: 61
  synthetic construct; capsid sequence

SEQ ID NO: 95 - protein, 734 aa, Artificial Sequence; identical to SEQ ID NO: 62
  synthetic construct; capsid sequence

SEQ ID NO: 96 - protein, 738 aa, Artificial Sequence
  synthetic construct; capsid sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 97 - protein, 746 aa, Artificial Sequence
  synthetic construct; capsid sequence
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNLALGETTRPATAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 98 - protein, 746 aa, Artificial Sequence
  synthetic construct; capsid sequence
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGALALGETTRPASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 99 - protein, 746 aa, Artificial Sequence
  synthetic construct; capsid sequence
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGLALGETTRPATTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 100 - protein, 748 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNLALGETTRPATAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 101 - protein, 748 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTLALGETTRPANDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 102 - protein, 748 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGLALGETTRPAGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 103 - protein, 748 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNLALGETTRPATAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 104 - protein, 748 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGALALGETTRPATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 105 - protein, 748 aa, Artificial Sequence
  synthetic construct; capsid sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTLALGETTRPATGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 106 - protein, 6 aa, Artificial Sequence
  synthetic construct; capsid sequence
HAIYPR

SEQ ID NO: 107 - protein, 700 aa, Artificial Sequence
  synthetic construct; capsid sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPE